(19) United States
(12) Patent Application Publication
(10) Pub. No.: US 2025/0074981 A1
(43) Pub. Date: Mar. 6, 2025

(54) COMBINATION THERAPY WITH DEXAMETHASONE AND TUMOR-SPECIFIC T CELL ENGAGING MULTI-SPECIFIC ANTIBODIES FOR TREATING CANCER

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Nai-Kong V. CHEUNG, New York, NY (US); Jeong PARK, New York, NY (US); Hong XU, New York, NY (US)

(21) Appl. No.: 18/554,184

(22) PCT Filed: Apr. 5, 2022
(86) PCT No.: PCT/US22/23473

§ 371 Date: Oct. 5, 2023

Related U.S. Application Data
(60) Provisional application No. 63/171,304, filed on Apr. 6, 2021.

(51) Int. Cl. C07K 16/28; A61K 31/573; A61K 39/00; A61K 45/06; A61P 35/00

(52) U.S. Cl. CPC C07K 16/2809; A61K 31/573; A61K 45/06; A61P 35/00; A61K 2039/505; A61K 2039/545; C07K 2317/31; C07K 2317/565; C07K 2317/622

(57) ABSTRACT

The present disclosure provides methods for treating cancer and/or delaying or decreasing cytokine release syndrome in a patient in need thereof comprising administering an effective amount of dexamethasone and an effective amount of tumor-specific T cell engaging multi-specific antibodies. Kits for use in practicing the methods are also provided.

### CROSS-REFERENCE TO RELATED APPLICATIONS

This application is the U.S. National Phase Application of International Application Number PCT/US2022/023473, filed Apr. 5, 2022, which claims the benefit of and priority to U.S. Provisional Patent Application No. 63/171,304, filed Apr. 6, 2021, the entire contents of each of which are incorporated herein by reference.

### STATEMENT OF GOVERNMENT SUPPORT

This invention was made with government support under grant number CA008748 awarded by National Cancer Institute. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 3, 2023, is named 115872-2514_SL.txt and is 385,844 bytes in size.

### TECHNICAL FIELD

The present technology relates generally to methods for treating cancer and/or delaying or decreasing cytokine release syndrome in a patient in need thereof comprising administering an effective amount of dexamethasone and an effective amount of tumor-specific T cell engaging multi-specific antibodies. Kits for use in practicing the methods are also provided.

### BACKGROUND

The following description of the background of the present technology is provided simply as an aid in understanding the present technology and is not admitted to describe or constitute prior art to the present technology.

The tumor microenvironment (TME) is a dynamic cellular milieu interacting with cancer cells that promote tumor growth, proliferation, angiogenesis, and metastasis, as well as regulating the tumor response to different types of immunotherapy. The immune hostile TME obstructs T cells driven by bispecific antibodies (BsAbs) or chimeric antigen receptor (CAR) from infiltrating into tumors, surviving, and fighting cancer cells. The TME plays an important role in tumor development, growth, and metastasis. The reciprocal interactions between cancer cells and TME promote cancer cell stemness and metabolic derangement and create a hypoxic and acidic environment. This immune-hostile TME reduces the viability and function of immune cells and supports stromal remodeling and angiogenesis by recruiting myeloid-derived suppressor cells (MDSCs), concomitantly protecting tumor cells from the host immune system and immunotherapy.

Accordingly, there is an urgent need for therapeutic compositions and methods that can overcome the immune-hostile TME and improve anti-tumor response of T cell-based cancer immunotherapies.

### SUMMARY OF THE PRESENT TECHNOLOGY

In one aspect, the present disclosure provides a method for treating cancer or inhibiting tumor growth in a subject in need thereof comprising administering to the subject an effective amount of dexamethasone and an effective amount of an anti-CD3 multi-specific antibody, wherein the anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the anti-CD3 multi-specific antibody is an immunoglobulin comprising two heavy chains and two light chains, wherein each of the light chains is fused to a single chain variable fragment (scFv).

In another aspect, the present disclosure provides a method for reducing or delaying cytokine release syndrome (CRS) in a subject in need thereof comprising administering to the subject an effective amount of dexamethasone and an effective amount of an anti-CD3 multi-specific antibody, wherein the anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the anti-CD3 multi-specific antibody is an immunoglobulin comprising two heavy chains and two light chains, wherein each of the light chains is fused to a single chain variable fragment (scFv).

In one aspect, the present disclosure provides a method for treating cancer or ameliorating cytokine release syndrome in a subject in need thereof comprising (a) administering to the subject a first effective dose of dexamethasone, (b) administering to the subject a first effective amount of an anti-CD3 multi-specific antibody about 1 hour after administration of the first effective dose of dexamethasone, (c) administering to the subject a second effective dose of dexamethasone about 72-96 hours after administration of the first effective dose of dexamethasone, (d) administering to the subject a second effective amount of the anti-CD3 multi-specific antibody about 1 hour after administration of the second effective dose of dexamethasone, and (e) repeating steps (a)-(d) for at least one additional cycle, wherein the anti-CD3 multi-specific antibody includes a CD3 binding domain comprising a heavy chain immunoglobulin variable domain (V_{H}) and a light chain immunoglobulin variable domain (V_{L}), wherein (i) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (ii) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the anti-CD3 multi-specific antibody is an immunoglobulin comprising two heavy chains and two light chains, wherein each of the light chains is fused to a single chain variable fragment (scFv). In another aspect, the present disclosure provides a method for treating cancer or ameliorating cytokine release syndrome in a subject in need thereof comprising (a) administering to the subject a first effective dose of dexamethasone, (b) administering to the subject a first effective amount of an ex vivo armed T cell that is coated or complexed with an effective arming dose of at least one type of anti-CD3 multi-specific antibody about 1 hour after administration of the first effective dose of dexamethasone, (c) administering to the subject a second effective dose of dexamethasone about 72-96 hours after administration of the first effective dose of dexamethasone, (d) administering to the subject a second effective amount of the ex vivo armed T cell about 1 hour after administration of the second effective dose of dexamethasone, and (e) repeating steps (a)-(d) for at least one additional cycle, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (i) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (ii) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the anti-CD3 multi-specific antibody is an immunoglobulin comprising two heavy chains and two light chains, wherein each of the light chains is fused to a single chain variable fragment (scFv). The first second effective dose and second effective dose of dexamethasone may be identical or different. In some embodiments, the first and/or second effective dose of dexamethasone is about 0.1 mg/kg-about 0.15 mg/kg, about 0.16 mg/kg-about 0.2 mg/kg, about 0.2 mg/kg-about 0.25 mg/kg, about 0.26 mg/kg-about 0.3 mg/kg, about 0.3 mg/kg-about 0.35 mg/kg, about 0.36 mg/kg-about 0.4 mg/kg, about 0.4 mg/kg-about 0.45 mg/kg, about 0.46 mg/kg-about 0.5 mg/kg, about 0.5 mg/kg-about 0.55 mg/kg, about 0.56 mg/kg-about 0.6 mg/kg, about 0.6 mg/kg-about 0.65 mg/kg, about 0.66 mg/kg-about 0.7 mg/kg, about 0.7 mg/kg-about 0.75 mg/kg, about 0.76 mg/kg-about 0.8 mg/kg, about 0.8 mg/kg-about 0.85 mg/kg, about 0.86 mg/kg-about 0.9 mg/kg, about 0.9 mg/kg-about 0.95 mg/kg, about 0.96 mg/kg-about 1 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, or about 35 mg/kg. In some embodiments, the subject is human and the first and/or second effective dose of dexamethasone is from about 0.16 mg/kg to about 8 mg/kg.

In one aspect, the present disclosure provides a method for treating cancer or inhibiting tumor growth in a subject in need thereof comprising administering to the subject an effective amount of dexamethasone and an effective amount of an ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the at least one type of anti-CD3 multi-specific antibody is an immunoglobulin comprising two heavy chains and two light chains, wherein each of the light chains is fused to a single chain variable fragment (scFv).

In another aspect, the present disclosure provides a method for reducing or delaying cytokine release syndrome (CRS) in a subject in need thereof comprising administering to the subject an effective amount of dexamethasone and an effective amount of an ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein the at least one type of anti-CD3 multi-specific antibody is an immunoglobulin comprising two heavy chains and two light chains, wherein each of the light chains is fused to a single chain variable fragment (scFv).

In any of the preceding embodiments of the methods disclosed herein, the ex vivo armed T cell is or has been cryopreserved. In certain embodiments, the ex vivo armed T cell has been cryopreserved for a period of about 2 hours to about 6 months. The ex vivo armed T cell may be a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell. Additionally or alternatively, in some embodiments of the methods disclosed herein, the ex vivo armed T cell is autologous, non-autologous, or derived in vitro from lymphoid progenitor cells.

Additionally or alternatively, in some embodiments of the methods disclosed herein, the at least one type of anti-CD3 multi-specific antibody exhibits surface densities between about 500 to about 20,000 molecules per T cell. In some embodiments, the effective arming dose of the at least one type of anti-CD3 multi-specific antibody is between about 0.05 μg/10⁶ T cells to about 5 μg/10⁶ T cells. In certain embodiments, the methods of the present technology further comprise administering a cytokine to the subject, optionally wherein the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2. The cytokine may be administered prior to, during, or subsequent to administration of the ex vivo armed T cell.

In any and all embodiments of the methods disclosed herein, at least one scFv of the anti-CD3 multi-specific antibody or at least one scFv of the at least one type of anti-CD3 multi-specific antibody comprises the CD3 binding domain. Additionally or alternatively, in some embodiments, at least one scFv of the anti-CD3 multi-specific antibody or at least one scFv of the at least one type of anti-CD3 multi-specific antibody comprises a DOTA binding domain. In certain embodiments, the DOTA binding domain comprises the amino acid sequence of any one of SEQ ID NOs: 77-80.

In any and all embodiments of the methods disclosed herein, the anti-CD3 multi-specific antibody or the at least one type of anti-CD3 multi-specific antibody binds one or more additional target antigens. Examples of additional target antigens include, but are not limited to CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), and a DOTA-based hapten.

Additionally or alternatively, in some embodiments of the methods disclosed herein, the V_{H} of the CD3 binding domain comprises the amino acid sequence of any one of SEQ ID NOs: 7-32, and/or the V_{L} of the CD3 binding domain comprises the amino acid sequence of any one of SEQ ID NOs: 33-70. In some embodiments, the anti-CD3 multi-specific antibody or the at least one type of anti-CD3 multi-specific antibody comprises a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, or a variant thereof having one or more conservative amino acid substitutions, and/or a light chain (LC) amino acid sequence comprising SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, or a variant thereof having one or more conservative amino acid substitutions.

In any of the preceding embodiments of the methods disclosed herein, the anti-CD3 multi-specific antibody or the at least one type of anti-CD3 multi-specific antibody comprises a HC amino acid sequence and a LC amino acid sequence selected from the group consisting of: SEQ ID NO: 82 and SEQ ID NO: 81, SEQ ID NO: 84 and SEQ ID NO: 83, SEQ ID NO: 86 and SEQ ID NO: 85, SEQ ID NO: 88 and SEQ ID NO: 87, SEQ ID NO: 90 and SEQ ID NO: 89, SEQ ID NO: 92 and SEQ ID NO: 91, SEQ ID NO: 94 and SEQ ID NO: 93, SEQ ID NO: 96 and SEQ ID NO: 95, SEQ ID NO: 98 and SEQ ID NO: 97, SEQ ID NO: 100 and SEQ ID NO: 99, SEQ ID NO: 102 and SEQ ID NO: 101, SEQ ID NO: 104 and SEQ ID NO: 103, SEQ ID NO: 106 and SEQ ID NO: 105, SEQ ID NO: 108 and SEQ ID NO: 107, SEQ ID NO: 110 and SEQ ID NO: 109, SEQ ID NO: 112 and SEQ ID NO: 111, SEQ ID NO: 114 and SEQ ID NO: 113, SEQ ID NO: 116 and SEQ ID NO: 115, SEQ ID NO: 118 and SEQ ID NO: 117, SEQ ID NO: 120 and SEQ ID NO: 119, SEQ ID NO: 122 and SEQ ID NO: 121, SEQ ID NO: 124 and SEQ ID NO: 123, SEQ ID NO: 126 and SEQ ID NO: 125, SEQ ID NO: 128 and SEQ ID NO: 127, SEQ ID NO: 130 and SEQ ID NO: 129, SEQ ID NO: 132 and SEQ ID NO: 131, SEQ ID NO: 134 and SEQ ID NO: 133, SEQ ID NO: 136 and SEQ ID NO: 135, SEQ ID NO: 138 and SEQ ID NO: 137, SEQ ID NO: 140 and SEQ ID NO: 139, SEQ ID NO: 142 and SEQ ID NO: 141, SEQ ID NO: 144 and SEQ ID NO: 143, SEQ ID NO: 146 and SEQ ID NO: 145, SEQ ID NO: 148 and SEQ ID NO: 147, and SEQ ID NO: 150 and SEQ ID NO: 149, respectively.

Additionally or alternatively, in some embodiments of the methods disclosed herein, the anti-CD3 multi-specific antibody or the at least one type of anti-CD3 multi-specific antibody comprise a first LC amino acid sequence, a first HC amino acid sequence, a second LC amino acid sequence, and a second HC amino acid sequence selected from the group consisting of SEQ ID NO: 99, SEQ ID NO: 100, SEQ ID NO: 101, and SEQ ID NO: 102; SEQ ID NO: 103, SEQ ID NO: 104, SEQ ID NO: 105, and SEQ ID NO: 106; SEQ ID NO: 107, SEQ ID NO: 108, SEQ ID NO: 109, and SEQ ID NO: 110; SEQ ID NO: 111, SEQ ID NO: 112, SEQ ID NO: 113, and SEQ ID NO: 114; SEQ ID NO: 115, SEQ ID NO: 116, SEQ ID NO: 117, and SEQ ID NO: 118; SEQ ID NO: 119, SEQ ID NO: 120, SEQ ID NO: 121, and SEQ ID NO: 122; SEQ ID NO: 123, SEQ ID NO: 124, SEQ ID NO: 125, and SEQ ID NO: 126; SEQ ID NO: 127, SEQ ID NO: 128, SEQ ID NO: 129, and SEQ ID NO: 130; SEQ ID NO: 131, SEQ ID NO: 132, SEQ ID NO: 133, and SEQ ID NO: 134; SEQ ID NO: 135, SEQ ID NO: 136, SEQ ID NO: 137, and SEQ ID NO: 138; SEQ ID NO: 139, SEQ ID NO: 140, SEQ ID NO: 141, and SEQ ID NO: 142; SEQ ID NO: 143, SEQ ID NO: 144, SEQ ID NO: 145, and SEQ ID NO: 146; and SEQ ID NO: 147, SEQ ID NO: 148, SEQ ID NO: 149, and SEQ ID NO: 150; respectively.

In any and all embodiments of the methods disclosed herein, the ex vivo armed T cell or the anti-CD3 multi-specific antibody is administered intravenously, intraperitoneally, subcutaneously, intramuscularly, or intratumorally. Additionally or alternatively, in some embodiments of the methods disclosed herein, the dexamethasone is administered intravenously, intraperitoneally, subcutaneously, intramuscularly, or intratumorally.

In some embodiments of the methods disclosed herein, the dexamethasone is administered about one hour prior to administration of the ex vivo armed T cell or the anti-CD3 multi-specific antibody. In other embodiments, the dexamethasone is administered about 24 hours prior to administration of the ex vivo armed T cell or the anti-CD3 multi-specific antibody.

Additionally or alternatively, in some embodiments, the methods of the present technology further comprise separately, simultaneously, or sequentially administering an additional cancer therapy. Examples of additional cancer therapies include, but are not limited to, chemotherapy, radiation therapy, immunotherapy, monoclonal antibodies, anti-cancer nucleic acids or proteins, anti-cancer viruses or microorganisms, and any combinations thereof. In some embodiments, the additional cancer therapy is an immune checkpoint inhibitor selected from among pembrolizumab, nivolumab, cemiplimab, atezolizumab, avelumab, durvalumab, and ipilimumab. In other embodiments, the additional cancer therapy comprises one or more of an anti-Ly6G antibody, an anti-GR-1 antibody, an anti-Ly6C antibody, an anti-CSF-1R antibody, Clodronate, a VEGF inhibitor and a VEGFR inhibitor.

In any and all embodiments of the methods disclosed herein, the subject is diagnosed with, or is suspected of having cancer. The cancer or tumor may be a carcinoma, sarcoma, a melanoma, or a hematopoietic cancer. Examples of cancer include, but are not limited to, osteosarcoma, Ewing's sarcoma, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof.

Also disclosed herein are kits containing components suitable for treating cancer in a patient. In certain embodiments, the kit comprises any and all embodiments of the anti-CD3 multi-specific antibody disclosed herein in unit dosage form, dexamethasone, and instructions for using the same to treat cancer. In certain embodiments, the kits further comprise instructions for arming T cells with the anti-CD3 multi-specific antibody. Additionally or alternatively, in some embodiments, the kits may further comprise instructions for isolating T cells from an autologous or non-autologous donor, and agents for culturing, differentiating and/or expanding isolated T cells in vitro such as cell culture media, CD3/CD28 beads, zoledronate, cytokines such as IL-2, IL-15 (e.g., IL15Rα-IL15 complex), buffers, diluents, excipients, and the like. Additionally or alternatively, in some embodiments, the kits comprise any and all embodiments of the EATs described herein, dexamethasone, and instructions for using the same to treat cancer in a subject in need thereof.

### BRIEF DESCRIPTION OF THE DRAWINGS

FIGS. 1A-1C show that treatment with T cells armed ex vivo (EAT) with tumor antigen×CD3 BsAb affects tumor infiltrating myeloid cell populations. FIG. 1A shows (upper) treatment schedule of GD2(+) patient-derived xenograft (PDX) tumors with GD2-EATs, and (lower) the effects on tumor volume over time. 20×10⁶ GD2-EATs regressed tumors, whereas other treatments did not work. FIG. 1B shows FACS results demonstrating that GD2-EATs more significantly depleted CD11b⁺Ly6G⁺F4/80⁻ granulocytic myeloid-derived suppressor cells (G-MDSCs), but not CD11b⁺Ly6G⁻F4/80⁺ monocytic MDSCs (M-MDSCs) and tumor associated macrophages (TAMs) in tumors. FIG. 1C shows FACS results demonstrating that relapsed tumors after GD2-EAT treatment were composed of a majority of mCD45⁺CD11b⁺Ly6G⁻ F4/80+ TAMs and minority of mCD45⁺CD11b⁺Ly6G⁺ F4/80⁻ G-MDSCs.

FIGS. 2A-2H show that granulocytes depletion modulates T cell homing into target tumor tissue and improves anti-tumor response of T cells armed with tumor antigen×CD3 BsAbs. FIG. 2A shows treatment schedule of G-MDSC depletion and GD2-EAT therapy. FIG. 2B shows complete blood count (CBC) analyses confirming the effect of anti-Ly6G antibody and anti-Gr-1 antibody on leukocyte counts in the blood. FIG. 2C shows flow cytometry analyses confirming the effect of anti-Ly6G antibody and anti-Gr-1 antibody on tumor infiltrating myeloid cells. G-MDSCs were gated as mouse CD45⁺CD11b⁺Ly6G⁺ cells, M-MDSCs were gated as mouse CD45⁺CD11b⁺Ly6G⁻Ly6C^{hi} cells, and TAMs were gated as mouse CD45⁺CD11b⁺Ly6G⁻F4/80⁺ cells. FIG. 2D shows flow cytometry analyses, demonstrating the effect of anti-Ly6G antibody and anti-Gr-1 antibody on tumor infiltrating T cells. FIG. 2E shows immunohistochemical (IHC) staining of tumor sections by anti-human CD3 antibody (×200). CD3⁺ T cell numbers were counted and compared among treatment groups. FIG. 2F shows treatment schedule for small cell lung cancer cell line (NCI-N417) xenografts with luciferase transduced T cells [Luc(+) T cells] combined with GD2-BsAb and anti-Gr-1 or anti-Ly6G antibody. Bioluminescence of Luc(+) T cells infiltrated into tumors was monitored. The bioluminescence images on day 4 and quantitation of the bioluminescence are shown. FIG. 2G shows in vivo anti-tumor response by GD2-BsAb and G-MDSC depleting treatments against melanoma cell line (M14) xenografts. FIG. 2H shows in vivo anti-tumor effect of GD2-EATs and anti-Gr-1 or anti-Ly6G antibody combination against osteosarcoma cell line (143B) xenografts.

FIGS. 3A-3H show that monocytes depletion modulates T cell homing into targeted tumors and improves anti-tumor response of T cells armed with tumor antigen×CD3 BsAbs. FIG. 3A shows treatment schedule of M-MDSC depletion and GD2-EAT therapy. FIG. 3B shows CBC and flow cytometry analyses confirming the effect of anti-Ly6C antibody on leukocyte counts in the blood. FIG. 3C shows flow cytometry analyses confirming the effect of anti-Ly6C antibody on tumor infiltrating myeloid cells. FIG. 3D shows flow cytometry analyses, demonstrating the effect of anti-Ly6C antibody on tumor infiltrating T cells. FIG. 3E shows immunohistochemical (IHC) staining of tumors by anti-human CD3 antibody (×200). CD3⁺ T cell numbers were counted and compared among treatment groups. FIG. 3F shows the results of administration of luciferase transduced HER2-EATs [Luc (+) HER2-EATs] with anti-Ly6C antibody to treat HER2(+) osteosarcoma PDXs, HGSOS. The Bioluminescence images on day 4 and quantitation of the bioluminescence are shown. FIG. 3G shows in vivo anti-tumor effect of GD2-EATs and anti-Ly6G antibody combination against osteosarcoma cell line, 143B, xenografts. FIG. 3H shows in vivo anti-tumor response by GD2-EATs and anti-Ly6C antibody against osteosarcoma PDX, TEOSC1.

FIGS. 4A-4H show that macrophage depletion modulates T cell homing into tumors and improve anti-tumor response of T cells armed with tumor antigen×CD3 BsAbs. FIG. 4A shows that clodronate liposome (CL) and anti-CSF1R antibody successfully depleted macrophages in the liver and spleen. Livers were stained with immunofluorescence antibodies (green, murine CD68; blue, nuclei), and spleens were processed with immunohistochemical (IHC) staining using murine CD68 antibody. FIG. 4B shows IHC staining of human CD45(+) T cells in M14 tumor sections after treatment with T cells plus GD2-BsAb and CL (100× magnification) (upper) and IHC staining of human CD3(+) T cells in 143B tumor sections after treatment with GD2-EATs plus anti-CSF1R antibody (×200) (lower). FIG. 4C shows the effects of injection of luciferase transduced T cells [Luc(+) T cells] with CL to treat small cell lung cancer (SCLC) xenografts. The intensity of bioluminescence was quantified and compared among groups. FIG. 4D shows the effects of administration of Luc(+) HER2-EATs with anti-CSF-1R antibody to HER2(+) osteosarcoma PDXs, HGSOS. The bioluminescence of Luc(+) HER2-EATs infiltrated into tumors was monitored. FIG. 4E shows in vivo anti-tumor response of GD2-BsAb and CL against melanoma cell line xenografts. FIG. 4F shows in vivo anti-tumor effects of GD2-EATs plus CL against osteosarcoma cell line, 143B, xenograft model. FIG. 4G shows anti-tumor effects of GD2-EATs plus anti-CSF-1R against 143B xenografts. FIG. 4H shows anti-tumor effects of GD2-EATs plus anti-CSF1R antibody against osteosarcoma PDXs mouse model.

FIGS. 5A-5F shows that targeting VEGF/VEGFR modulates T cell homing into tumors and improves anti-tumor response of T cells armed with tumor antigen×CD3 BsAbs against hypoxic solid tumors. FIG. 5A shows flow cytometry analyses of tumor infiltrating lymphocytes on day 60 post treatment of GD2-EATs (10 g of GD2-BsAb/2×10⁷ cells/dose, 2 doses/week for 3 weeks) plus VEGF blockades (each 100 g/dose, 1 day prior to each GD2-EATs). FIG. 5B shows immunohistochemical (IHC) staining of tumor sections by anti-human CD3 antibody (200× magnification) and comparison of T cell count among groups. FIG. 5C shows the effects of administration of luciferase transduced HER2-EATs [Luc(+) HER2-EATs](on day 0) with VEGF blockades (day −1). Quantified bioluminescence intensities were compared. FIG. 5D shows in vivo anti-tumor response of GD2-EATs and VEGF blockades against osteosarcoma PDX, TEOSC1. FIG. 5E shows H&E staining of relapsed TEOSC1 tumor after GD2-EAT therapy and residual bony mass after treatment with GD2-EATs plus VEGF blockades. FIG. 5F shows the effects of administration of GD2-EATs and anti-VEGF or anti-VEGFR2 antibody combinations to treat osteosarcoma 143B xenografts, and the anti-tumor effects were compared among groups.

FIGS. 6A-6G show that dexamethasone depleted TAMs and increased T cell infiltration into tumors, improving anti-tumor effect of T cells armed with tumor antigen×CD3 BsAbs. FIG. 6A shows treatment schedule of dexamethasone premedication testing for anti-GD2 BsAb and T cell therapy (GD2-EATs or GD2-BsAb plus T cell injection). Three different doses of dexamethasone [low dose (LD), intermediate dose (ID), and high dose (HD) dexamethasone] were administered one hour prior to each BsAb and EAT therapy. FIG. 6B shows T helper cell cytokine levels analyzed 4 hours after the first dose of GD2-EATs. FIG. 6C shows the effects of flow cytometry analyses of the effect of dexamethasone premedication on leukocytes in the blood. FIG. 6D shows flow cytometry analyses of the effects of dexamethasone premedication on tumor infiltrating myeloid cells (TIMs) and infiltrating lymphocytes (TTLs). FIG. 6E shows immunohistochemical (IHC) staining of tumor sections by human CD3 antibody (×200). T cell numbers were compared among groups. FIG. 6F shows quantified bioluminescence intensities of Luc(+) GD2-EATs trafficked into tumors. FIG. 6G shows bioluminescence images of Luc(+) GD2-EATs in the lung on day 1 and in tumors on day 5.

FIGS. 7A-7B show that dexamethasone improved anti-tumor effect of T cells armed with tumor antigen×CD3 BsAbs. FIG. 7A shows in vivo anti-tumor effect of dexamethasone premedication on GD2-BsAb plus T cell therapy or GD2-EAT therapy in neuroblastoma PDX mouse model. Different doses of dexamethasone significantly affected neuroblastoma tumor growth and overall survival of mice treated with GD2-BsAb. FIG. 7B shows in vivo anti-tumor effect of intermediate or low dose dexamethasone premedication with GD2-EAT therapy in osteosarcoma PDX mouse model.

FIG. 8 shows exemplary amino acid sequences of anti-CD3 multi-specific antibodies that are useful in the methods of the present technology. Linker sequences are underlined.

FIG. 9A shows treatment schedule of dexamethasone premedication testing for anti-HER2-BsAb and T cell therapy (HER2-BsAb plus T cell injection). Three different doses of dexamethasone [low dose (LD), intermediate dose (ID), and high dose (HD) dexamethasone] were administered once a week for 3 weeks. FIG. 9B shows in vivo anti-tumor effect of dexamethasone premedication on HER2-BsAb plus T cell therapy in M37 breast cancer PDX mouse model. FIG. 9C shows a comparison of in vivo serum cytokine levels among treatment groups. FIGS. 9D-9E show a comparison of T cell engraftment among treatment groups after 1^{st} dose of T cells and 2^{nd} dose of T cells, respectively. FIG. 9F shows a comparison of CD4 to CD8 T cell ratio among the different treatment groups.

FIGS. 10A-10B demonstrate that tumor cells express VEGF, and anti-VEGF antibody reduced VEGF levels in the blood. FIG. 10A: Flow cytometry analysis of VEGF expression on varieties of tumor cell line was analyzed with increasing cell density after incubation for 48 hours. FIG. 10B: Mouse serum hVEGF levels were analyzed after treatment of VEGF blockades and anti-HER2-EAT therapy against HGSOC1 osteosarcoma PDXs.

FIGS. 11A-11B demonstrate that antiangiogenic therapies enhance BsAb-driven T cell trafficking and persistence in tumors. FIG. 11A: 1×10⁷ of Luciferase transduced T cells ex vivo armed with anti-HER2 BsAb [Luc(+) HER2-EATs]were administered with bevacizumab or DC101 to mice bearing HER2(+) HGSOC1 osteosarcoma PDXs, and the bioluminescence of T cells in tumors were analyzed over time and quantified as a bioluminescence intensity (BLI). FIG. 11B: Luc(+) GD2-EATs were administered with anti-VEGF or VEGFR2 antibody to treat GD2(+) neuroblastoma PDXs, and the bioluminescence of T cells in tumors were analyzed over time.

FIGS. 12A-12C demonstrate that antiangiogenic therapies enhance BsAb-driven T cell infiltration into solid tumors. FIG. 12A: Neuroblastoma PDXs treated by GD2-EATs with or without VEGF blockades were analyzed by flow cytometry on day 10 post-EAT treatment. FIG. 12B: 143B osteosarcoma cell line-derived xenografts (CDXs) were treated with a combination of GD2-EATs and anti-VEGF or VEGFR2 antibody, and tumors were analyzed by flow cytometry on day 60 post-EAT treatment. FIG. 12C: The 143B osteosarcoma CDXs harvested on day 60 post-treatment were immunohistochemical stained with anti-human CD3 antibody, and the CD3+ T cell numbers were compared among groups.

FIGS. 13A-13B demonstrate that antiangiogenic therapies increased intratumoral CD8+ T cell infiltration. FIG. 13A: Immunohistochemical staining (IHC) of tumor xenografts treated by GD2-EATs with or without VEGF blockades. Tumors were harvested on day 10 post-EAT treatment and stained with anti-human CD3, anti-human CD4, and anti-human CD8 antibody. FIG. 13B: Intratumoral CD3, CD4, and CD8 T cells were quantified by positive pixel analyses and compared among groups.

FIGS. 14A-14C demonstrate the effect of antiangiogenic therapies on tumor microvasculature. FIG. 14A: Neuroblastoma PDXs were harvested on day 10 post-EAT treatment, and intratumoral blood vessels were stained using mCD31 antibody. FIG. 14B: Combinations of GD2-EATs and anti-VEGF or anti-VEGFR2 antibody induced high endothelial venules (HEVs) in tumors. FIG. 14C: Neuroblastoma PDXs harvested on day 10 post-EAT treatment were immunohistochemical stained with anti-HIF1α antibody and compared among groups by positive pixel count analysis.

FIGS. 15A-15C demonstrate the in vivo anti-tumor effect of combination therapies of GD2-EATs and VEGF blockades against osteosarcomas. FIG. 15A: 143B osteosarcoma CDXs were treated by GD2-EATs with or without VEGF blockades, and in vivo tumor suppressing effects and overall survival were analyzed and compared among groups. FIG. 15B: TEOSC1 osteosarcoma PDXs were treated by combinations of GD2-EATs and VEGF blockades. Tumor growth curves were plotted and compared with control groups (no treatment, unarmed T cells, or GD2-EATs alone). FIG. 15C: H&E staining of tumors harvested on day 180 post-treatment. G3, GD2-EATs; G4, GD2-EATs plus anti-VEGF antibody; G5, GD2-EATs plus anti-VEGFR2 antibody.

FIGS. 16A-16B demonstrate the in vivo anti-tumor effect of combination therapies of EATs and VEGF blockades. FIG. 16A: Synergistic anti-tumor effect between GD2-EATs and VEGF blockades were tested in a neuroblastoma PDX model. Tumor growth and overall survival curves were plotted and compared among groups. FIG. 16B: In vivo anti-tumor effect of combinations of GPC3-EATs and VEGF blockades were tested using HepG2 hepatoblastoma and compared among groups.

FIGS. 17A-17B demonstrate the effect of VEGF/VEGFR inhibitors on TH1 cell cytokine levels after EAT therapy. FIG. 17A: Mouse serum human TH1 cell cytokine levels were measured 2 hours after the first dose of EAT injection. FIG. 17B: Serum TH1 cell cytokine levels were analyzed over time.

FIG. 18 shows monitoring of bioluminescence of luciferase-transduced GD2-EATs in tumors over time.

FIGS. 19A-19B demonstrate the effect of anti-antigenic therapies on complete blood counts and tumor infiltrating myeloid cells. FIG. 19A: GD2-EATs with or without VEGF blockades were administered to treat neuroblastoma PDXs, and complete blood count were analyzed on day 5 post-EAT treatment. FIG. 19B: Tumors were harvested on day 10 post-EAT treatment, and tumor infiltrating myeloid cells (TIMs) were analyzed and compared among groups.

FIGS. 20A-20B demonstrate the effect of anti-antigenic therapies on complete blood counts and tumor infiltrating myeloid cells. FIG. 20A: GD2-EATs with or without VEGF blockades were administered to treat 143B osteosarcoma CDXs, and complete blood count were analyzed on day 5 post-EAT treatment. FIG. 20B: Tumors were harvested on day 60 post-EAT treatment, and tumor infiltrating myeloid cells (TIMs) were analyzed and compared among groups.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

T cells driven by bispecific antibodies or chimeric antigen receptors release cytokines that create a life threatening cytokine release syndrome (CRS) that often limits dose escalation and complicates patient management. The present disclosure demonstrates that a bolus of high dose dexamethasone (e.g., 2 mg/kg, or 8 mg/kg or 32 mg/kg) prior to each injection of an anti-tumor T cell engaging bispecific antibody decreased or delayed cytokine release. Moreover, the combination therapy methods disclosed herein deplete MDSC and especially macrophage lineage MDSC, thus improving CD8(+) T cell infiltration and significantly improving anti-tumor response. These results were unexpected given the literature describing the adverse effects of corticosteroids on T cell functions. For example, in one preclinical model, simultaneous administration of corticosteroids impaired antitumor responses with reduction of CD8+ T cell proliferation, and decreased low- but not high-affinity memory T cells by suppressing fatty acid metabolism essential for memory T cells (Tokunaga A et al., Journal of Experimental Medicine 216:2701-2713 (2019)). Genome-wide expression suggested that glucocorticoids largely spare or enhance gene pathways that are associated with innate immunity, but selectively suppress pathways that are involved in adaptive immunity. A biphasic dose-response curve for glucocorticoids has been proposed, being ‘permissive’ (that is, immunostimulatory) at low concentrations and suppressive at high concentrations (Munck A, Naray-Fejes-Toth A, Mol Cell Endocrinol 90:C1-4 (1992); Sapolsky R M, Romero L M, Munck A U, Endocr Rev 21:55-89, (2000)). Accordingly, the combination therapy methods disclosed herein are effective in decreasing or delaying CRS and improving the efficacy of anti-tumor T cell engaging multi-specific antibody therapies in patients.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (k) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds CD3 protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multi-specific antibodies, bispecific antibodies, etc.,) as well as antigen binding fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi-specific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the F, fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain F, (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a CD3 polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

As used herein, an “armed T cell” refers to any white blood cell expressing CD3 on its cell surface that has been coated with one or more multi-specific antibodies (e.g., BsAbs) having antineoplastic and/or immunomodulating activities. By way of example only, but not by way of limitation, T cells may be expanded and/or activated ex vivo and then armed with an anti-CD3 multi-specific antibody (e.g., a BsAb). Upon administration, the multi-specific antibody-armed activated T cells are configured to localize to a tumor cell expressing a target antigen (e.g., tumor antigen) recognized by the anti-CD3 multi-specific antibody, and selectively cross-link with the tumor cells; this may result in the recruitment and activation of cytotoxic T lymphocytes (CTLs), CTL perforin-mediated tumor cell cytolysis, and/or the secretion of antitumor cytokines and chemokines.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Dosage form” and “unit dosage form”, as used herein, the term “dosage form” refers to physically discrete unit of a therapeutic agent for a subject (e.g., a human patient) to be treated. Each unit contains a predetermined quantity of active material calculated or demonstrated to produce a desired therapeutic effect when administered to a relevant population according to an appropriate dosing regimen. For example, in some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). It will be understood, however, that the total dosage administered to any particular patient will be selected by a medical professional (e.g., a medical doctor) within the scope of sound medical judgment.

“Dosing regimen” (or “therapeutic regimen”), as used herein is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in certain embodiments, a dosing regimen comprises a plurality of doses and at least two different time periods separating individual doses. In some embodiments, the therapeutic agent is administered continuously (e.g., by infusion) over a predetermined period. In other embodiments, a therapeutic agent is administered once a day (QD) or twice a day (BID). In some embodiments, a dosing regimen comprises a plurality of doses each of which are separated from one another by a time period of the same length; in other embodiments, a dosing regimen comprises a plurality of doses and at least two different time periods separating individual doses. In some embodiments, all doses within a dosing regimen are of the same unit dose amount. In certain embodiments, different doses within a dosing regimen are of different amounts. In some embodiments, a dosing regimen comprises a first dose in a first dose amount, followed by one or more additional doses in a second dose amount different from the first dose amount. In other embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (112) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 amino acid residues. In certain embodiments, the linker comprises amino acids having the sequence

(SEQ ID NO: 154)
GGGGSGGGGSGGGGS (i.e., [G₄S]₃),
(SEQ ID NO: 151)
GGGGSGGGGSGGGGSGGGGS (i.e., [G₄S]₄),
(SEQ ID NO: 152)
GGGGSGGGGSGGGGSGGGGSGGGGS (i.e., [G₄S]₅),
or
(SEQ ID NO: 153)
GGGGSGGGGSGGGGSGGGGSGGGGSGGGGS (i.e., [G₄S]₆)

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, PA.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a CD3 polypeptide), or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, “solid tumor” refers to all neoplastic cell growth and proliferation, and all pre-cancerous and cancerous cells and tissues, except for hematologic cancers such as lymphomas, leukemias, and multiple myeloma. Examples of solid tumors include, but are not limited to: soft tissue sarcoma, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor and other bone tumors (e.g., osteosarcoma, malignant fibrous histiocytoma), leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, brain/CNS tumors (e.g., astrocytoma, glioma, glioblastoma, childhood tumors, such as atypical teratoid/rhabdoid tumor, germ cell tumor, embryonal tumor, ependymoma), medulloblastoma, craniopharyngioma, ependymoma, pinealoma, hemangioblastoma, acoustic neuroma, oligodendroglioma, meningioma, melanoma, neuroblastoma, and retinoblastoma.

As used herein, the terms “subject”, “patient”, or “individual” can be an individual organism, a vertebrate, a mammal, or a human. In some embodiments, the subject, patient or individual is a human.

As used herein, the term “T cell” includes naïve T cells, CD4+ T cells, CD8+ T cells, memory T cells, activated T cells, anergic T cells, tolerant T cells, chimeric B cells, and antigen-specific T cells.

As used herein, the term “therapeutic agent” is intended to mean a compound that, when present in an effective amount, produces a desired therapeutic effect on a subject in need thereof.

As used herein “tumor-infiltrating lymphocytes” or “TILs” refer to white blood cells that have left the bloodstream and migrated into a tumor.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-CD3 antibodies described herein are contemplated. For example, it may be desirable to improve the binding affinity and/or other biological properties of the antibody. Amino acid sequence variants of an anti-CD3 antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 1
Amino Acid Substitutions

| Original | | Conservative |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

### TME

The TME is characterized by high infiltration of monocytes, macrophages, dendritic cells and granulocytes. These tumor-infiltrating myeloid cells (TIMs) constitute a heterogeneous population of cells characterized by diversity, plasticity, and immaturity and are potent mediators of immune suppression, tumor angiogenesis and metastases, being at the basis of treatment failure. In murine tumor models, TIMs are phenotyped as either ‘pro-tumoral’ M2 tumor-associated macrophages (TAMs) or myeloid-derived suppressor cells (MDSCs) by immunohistochemical staining (IHC). MDSCs can be further subdivided into 2 major groups: granulocytic MDSCs (G-MDSCs) and monocytic MDSCs (M-MDSCs). In mice G-MDSCs have a phenotype of CD11b⁺Ly6G⁺Ly6C^{low}, in contrast to M-MDSCs being CD11b⁺Ly6G-Ly6C^{high}, while mouse macrophages are characterized by the expression of CD11b and F4/80. MDSCs inhibit T cell metabolism by hoarding key amino acids, modulate T cell homing by cleaving L-selectin, and prevent T cell activation by increasing PD-L1 expression especially when hypoxic. Immunosuppressive M2 TAMs promote T cell anergy via increased nitric oxide (NO) and decreased arginine under hypoxic conditions. Another challenge for T cell immunotherapy is that most solid tumors have an immature and chaotic microvasculature responsible for the hypoxic TME, which in turn promote desmoplasia and inflammation, contributing to tumor progression and therapeutic resistance. Known negative immune modulators such as hypoxia, VEGF, Tregs, and inhibitory cytokines often converge through or derive from MDSC. Hence, strategies to remove or to turn off MDSCs are actively being tested in preclinical models. However, clinical trials using antibodies and small molecule inhibitors by themselves to modulate MDSC or TAM functions have been mostly unsuccessful thus far. (Haibe et al., Front Oncol 10:221 (2020); Hayes D F. JAMA 305(5):506-8 (2011); Hurwitz H et al., N Engl J Med 350(23):2335-42 (2004)).

### Corticosteroids

Corticosteroids refer to a class of steroids (lipids that contain a hydrogenated cyclopentoperhydrophenanthrene ring system) produced by the adrenal cortex (except sex hormones of adrenal origin) in response to the release of adrenocorticotrophin or adrenocorticotropic hormone by the pituitary gland, or to any synthetic equivalent, or to angiotensin II. Corticosteroids are characterized by mineralocorticoid and glucocorticoid effects, depending on the pharmacology of the agent. Mineralocorticoids are characterized by their similarity to aldosterone and their influence on electrolyte levels and water balance. The glucocorticoids, such as the endogenous glucocorticoid cortisol, control metabolism and are anti-inflammatory by preventing cytokine release.

Corticosteroids are immunosuppressive for both the innate and adaptive immunities (Oppong E, Cato A C B: Effects of Glucocorticoids in the Immune System, in Wang J-C, Harris C (eds): GLUCOCORTICOID SIGNALING: FROM MOLECULES TO MICE TO MAN. New York, NY, Springer New York, 2015, pp 217-233), and induce cell death in lymphocytes (Boldizsar F, Talaber G, Szabo M, et al., Immunobiology 215:521-6 (2010); Herold M J, McPherson K G, Reichardt H M, Cell Mol Life Sci 63:60-72 (2006)), dendritic cells (DCs) (Kim K D, Choe Y K, Choe I S, et al., J Leukoc Biol 69:426-34 (2001)), eosinophils (Druilhe A, Letuve S, Pretolani M: Apoptosis 8:481-95 (2003)), and osteocytes (Weinstein R S, Jilka R L, Parfitt A M, et al., J Clin Invest 102:274-82 (1998)). Glucocorticoids attenuate DC activity by decreasing DC numbers (Cao Y, Bender I K, Konstantinidis A K, et al., Blood 121:1553-62, 2013) and by inhibiting DC maturation through downregulation of the expression of MHC class II, the lipid-presentation molecule CDla, co-stimulatory molecules (CD80 and CD86) and proinflammatory cytokines (e.g. IL-12 and TNF); and by promoting the expression of anti-inflammatory cytokines, such as IL-10 (Szatmari I, Nagy L, Embo J 27:2353-62 (2008)). Glucocorticoids dampen T cell activation by interfering with TCR signaling, resulting in reduced proliferative responses and diminished cytokine production (e.g., IL2), especially for TH1 cells and TH17 cells, while sparing, or possibly even promoting, the function of TH2 cells and regulatory T (Treg) cells (Cain D W, Cidlowski J A, Nat Rev Immunol 17:233-247 (2017)). Steroids have been found to upregulate CTLA-4 mRNA and protein in both CD4(+) T cells and CD8(+) T cells, while blocking CD28-mediated cell cycle entry and differentiation in naive T cells (Giles A J, Hutchinson M-K N D, Sonnemann H M, et al., Journal for ImmunoTherapy of Cancer 6:51 (2018)).

Baseline use of corticosteroids is associated with poor outcomes in patients with non-small-cell lung cancer (NSCLC) treated with programmed cell death-I axis inhibition (Arbour K C, Mezquita L, Long N, et al., J Clin Oncol 36:2872-2878 (2018)). Patients treated with ≥10 mg of prednisone at the time of immunotherapy initiation have worse outcomes than patients who received 0 to <10 mg of prednisone (Ricciuti B, Dahlberg S E, Adeni A, et al., J Clin Oncol 37:1927-1934 (2019)). In mice dexamethasone decreased the number of NK cells in the spleen and suppressed their activity (Chen L, Jondal M, Yakimchuk K, Inflammopharmacology 26:1331-1338 (2018)). In particular, the expression of both Ly49G and NKG2D receptors was decreased. Dexamethasone given to patients with immunologically “cold” tumors or those with insufficient anti-tumor immunity could abrogate new priming and the adaptive anti-tumor T cell response. Arbour K C et al., J Clin Oncol 36:2872-2878 (2018); Ricciuti B et al., J Clin Oncol 37:1927-1934 (2019).

### T Cell Engaging Multi-specific Antibodies of the Present Technology

The T cell engaging multi-specific antibodies of the present technology include, e.g., but are not limited to, monoclonal, chimeric, humanized, bispecific antibodies, trispecific antibodies, or tetraspecific antibodies that specifically bind a CD3 target polypeptide, a homolog, derivative or a fragment thereof. In any and all embodiments of the multi-specific (e.g., bispecific) antibodies or EATs disclosed herein, the anti-CD3 multi-specific antibody is an immunoglobulin comprising two heavy chains and two light chains, wherein each of the light chains is fused to a single chain variable fragment (scFv). Such an anti-CD3 multi-specific antibody includes a CD3 binding domain comprising a heavy chain immunoglobulin variable domain (V_{H}) and a light chain immunoglobulin variable domain (V_{L}). Additionally or alternatively, in some embodiments, at least one scFv of the anti-CD3 multi-specific antibody disclosed herein comprises the CD3 binding domain. The CDR sequences of the V_{H} and V_{L} of the CD3 binding domain based on the IMGT annotation system are summarized below:

| Region | Definition | Sequence Fragment | Residues | Length |
|---|---|---|---|---|
| CDR-H1 | IMGT | GYTFTRYT (SEQ ID NO: 1) | 26-33 | 8 |
| CDR-H2 | IMGT | INPSRGYT (SEQ ID NO: 2) | 51-58 | 8 |
| CDR-H3 | IMGT | ARYYDDHYSLDY (SEQ ID NO: 3) | 97-108 | 12 |
| CDR-L1 | IMGT | SSVSY (SEQ ID NO: 4) | 27-31 | 5 |
| CDR-L2 | IMGT | DT (SEQ ID NO: 5) | 49-50 | 2 |
| CDR-L3 | IMGT | QQWSSNPFT (SEQ ID NO: 6) | 88-96 | 9 |

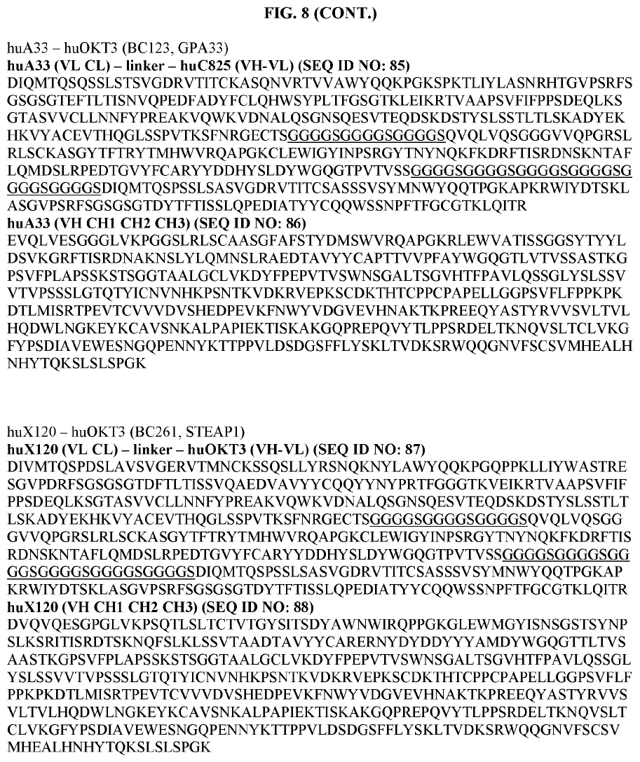

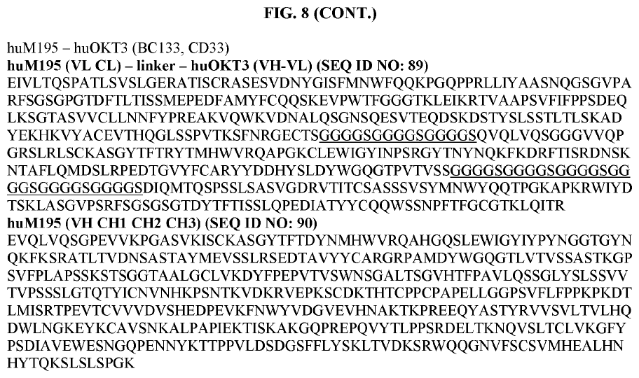

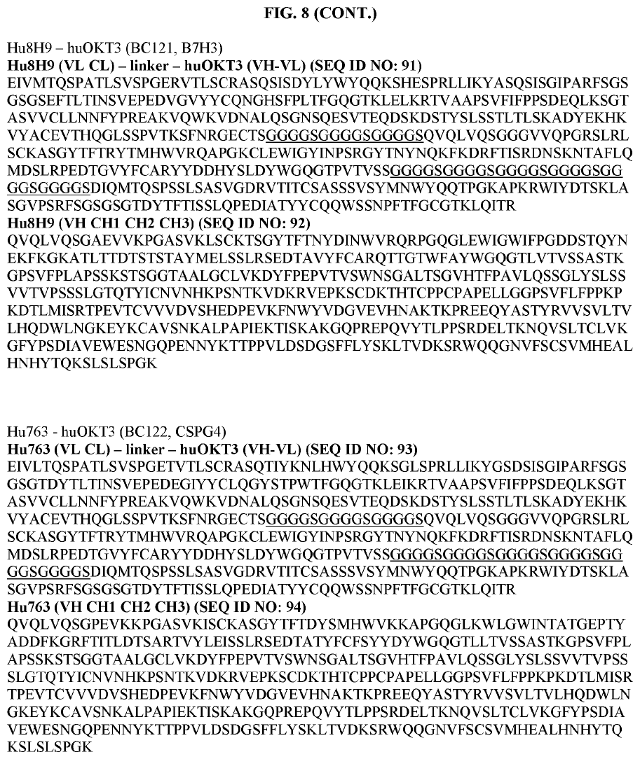

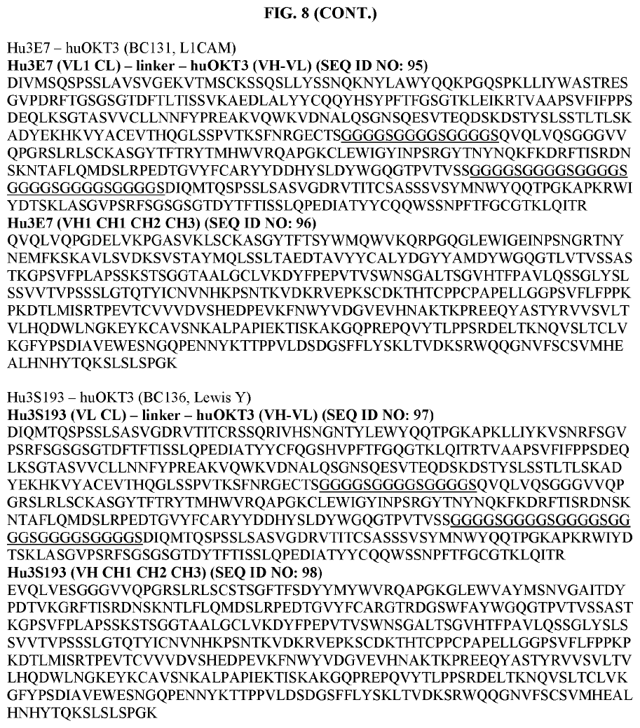

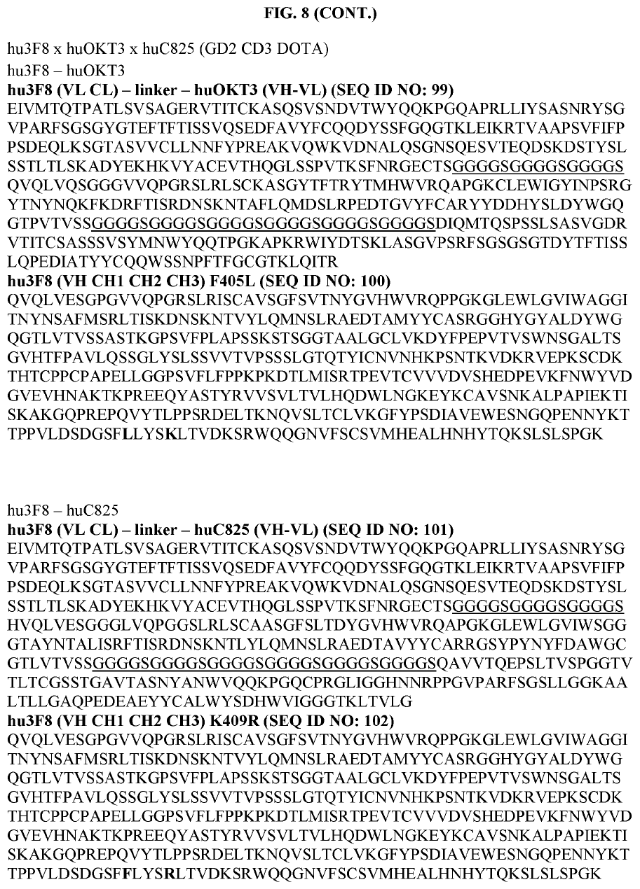

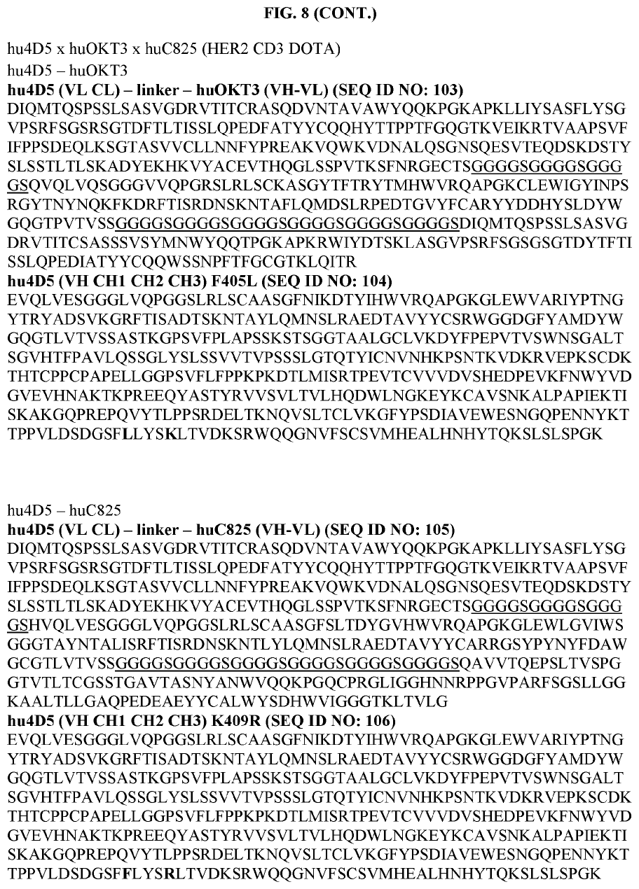

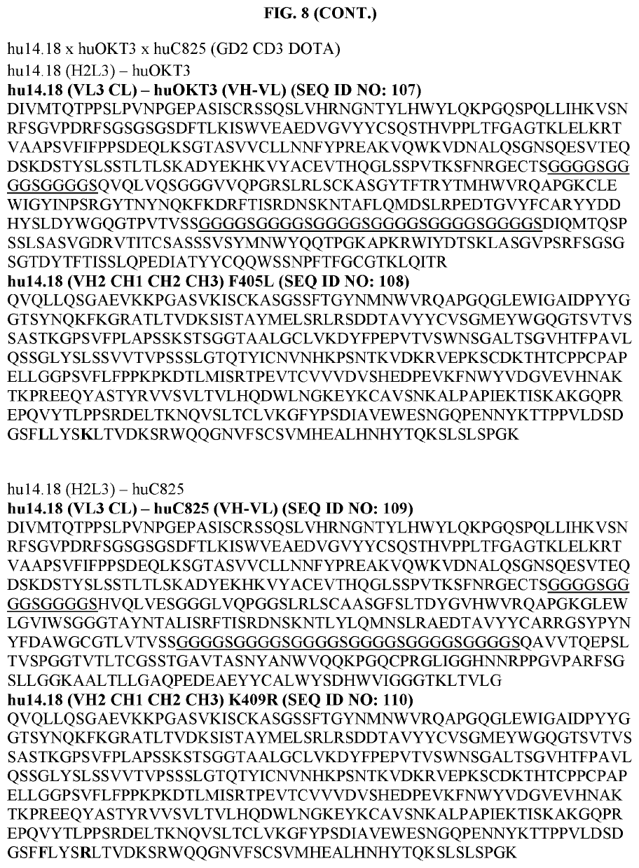

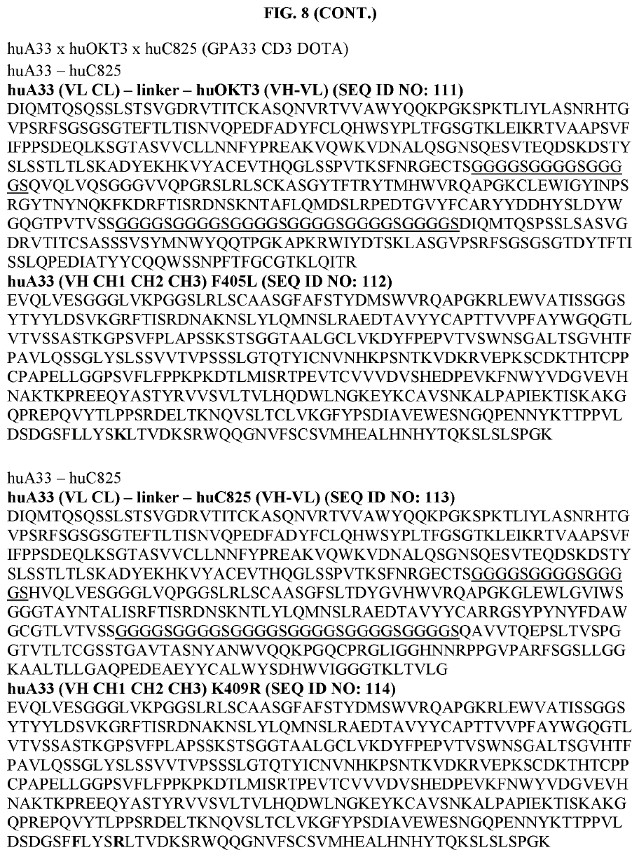

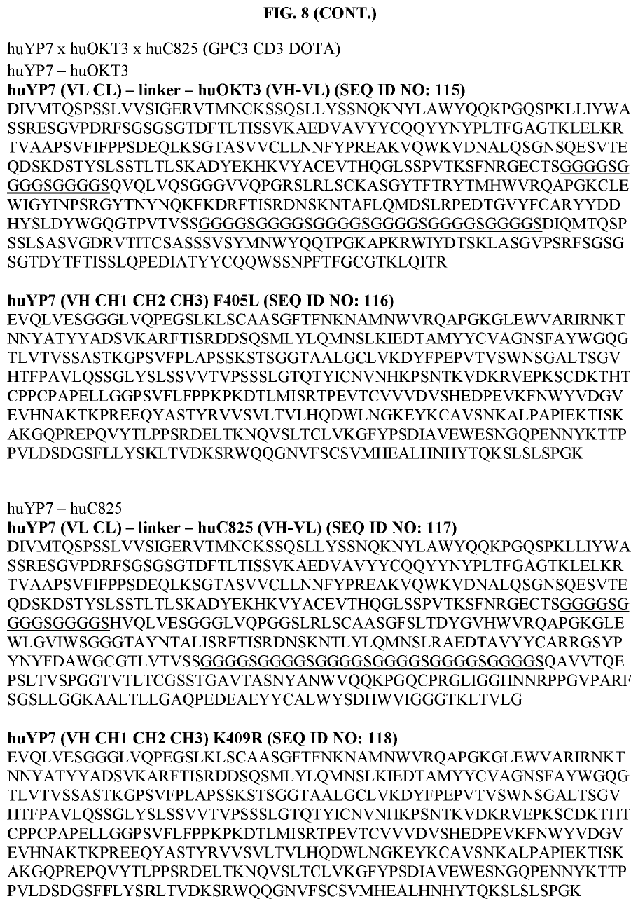

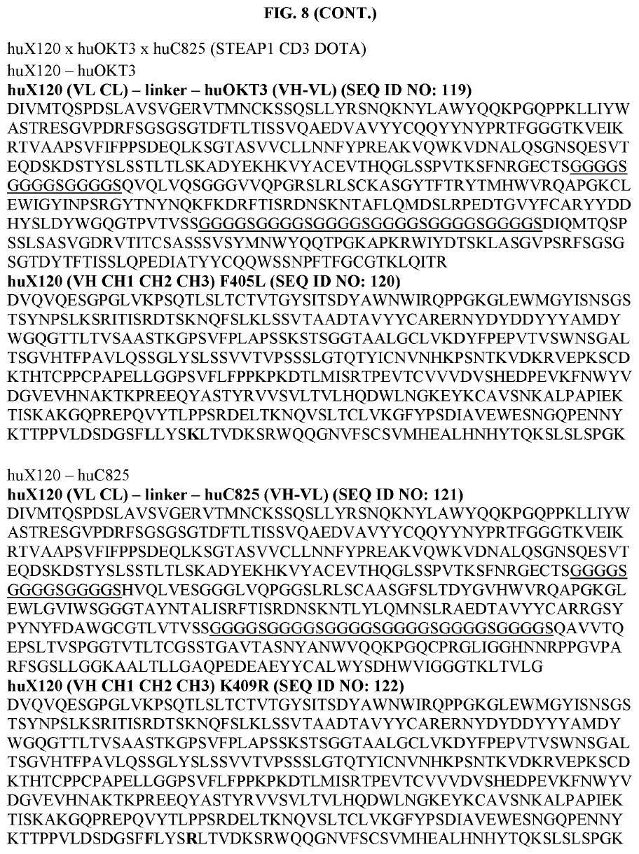

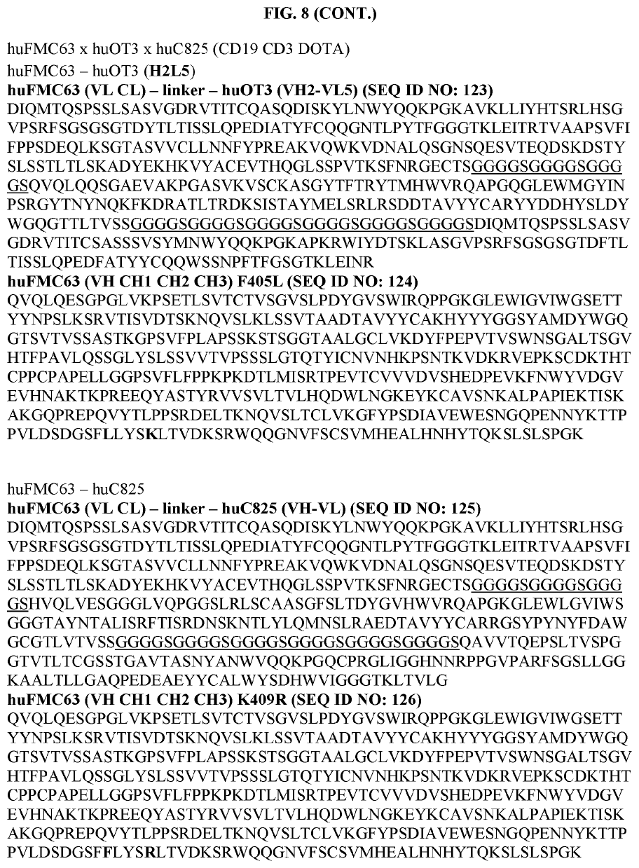

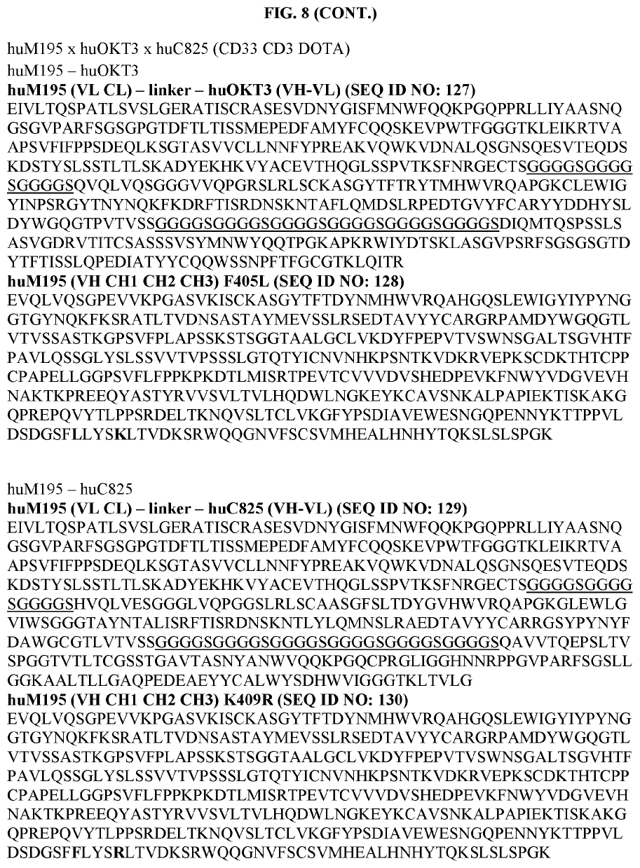

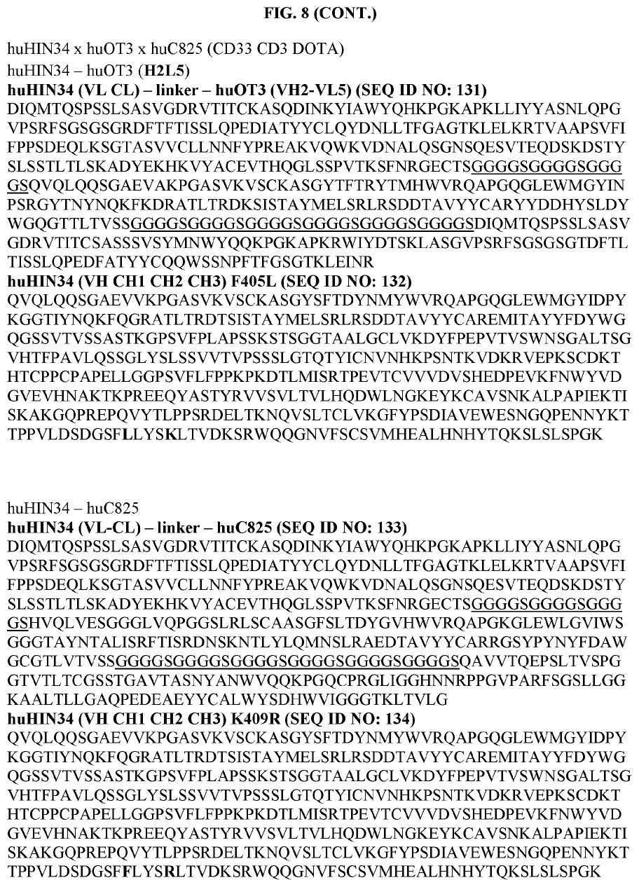

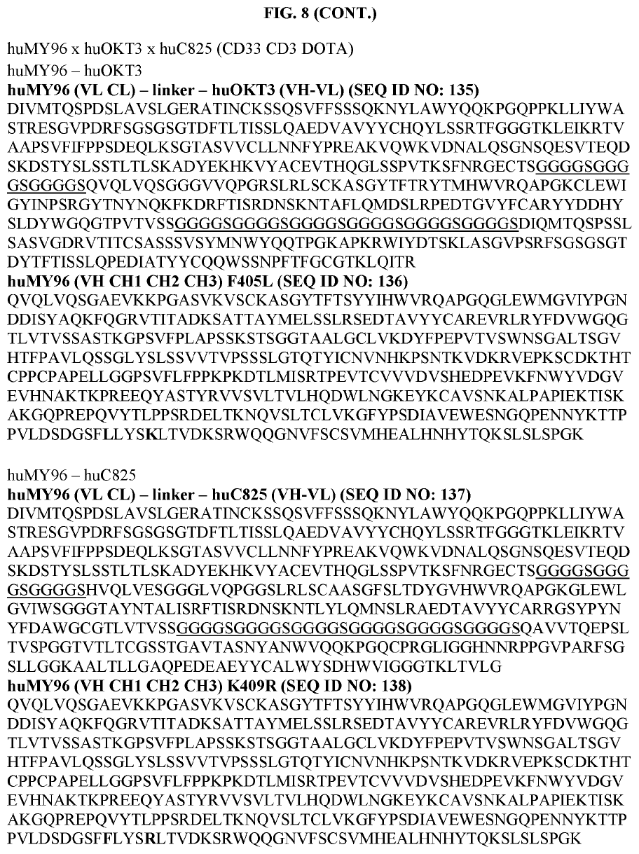

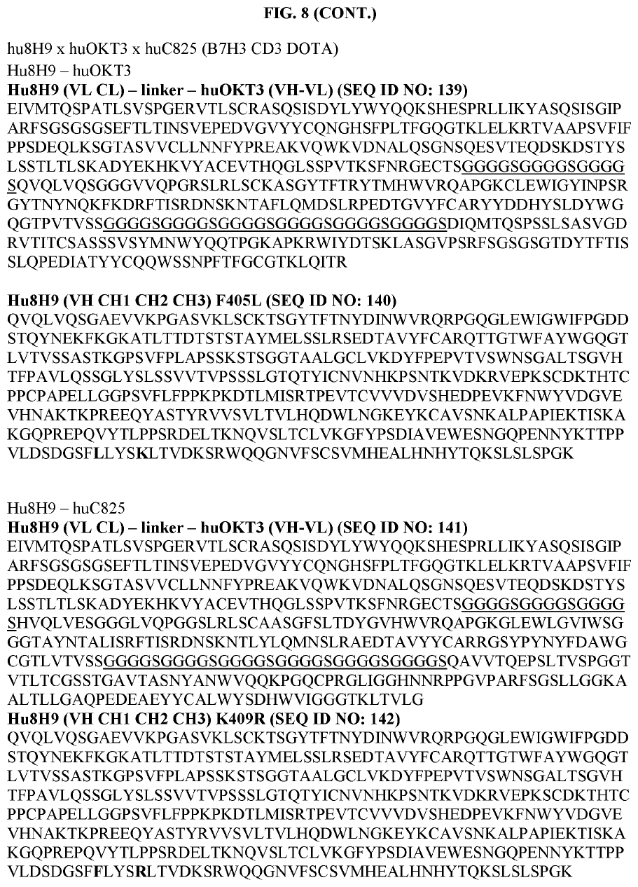

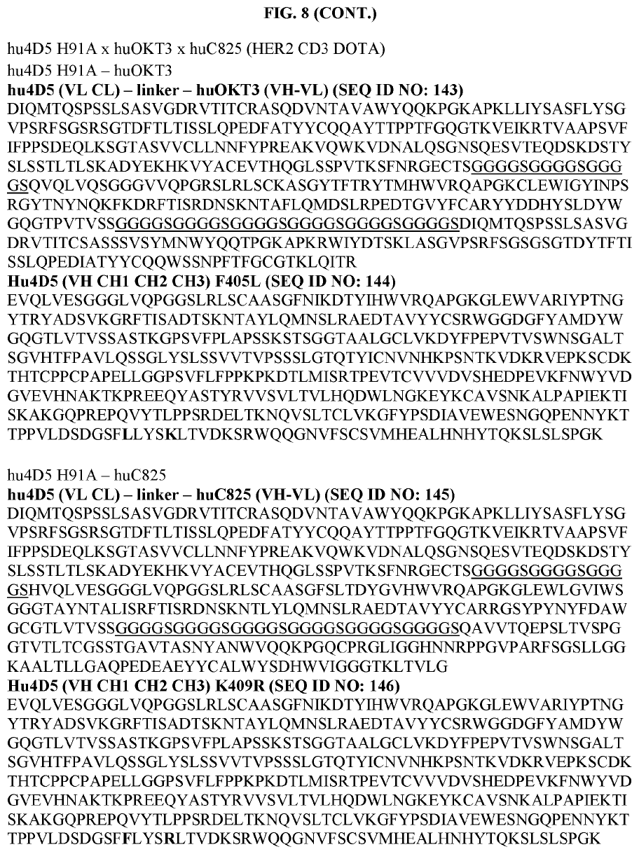

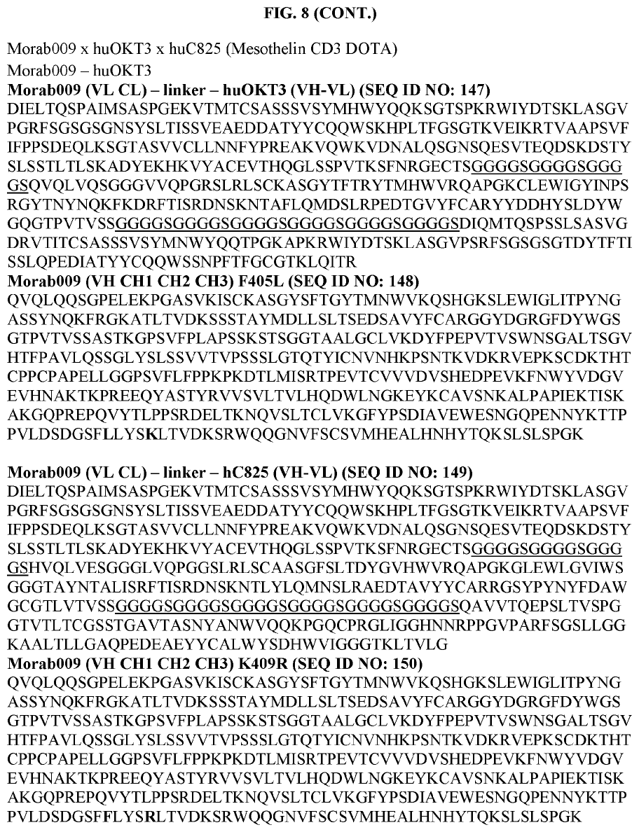

FIG. 8 shows exemplary amino acid sequences of anti-CD3 multi-specific antibodies that are useful in the methods of the present technology. In some embodiments, the anti-CD3 multi-specific antibodies disclosed in FIG. 8 may be used to arm the EATs of the present technology.

In some embodiments, the anti-CD3 multi-specific antibodies of the present technology include a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and/or (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6. Additionally or alternatively, in some embodiments, the anti-CD3 multi-specific antibodies may be used to arm the EATs of the present technology.

Exemplary heavy chain immunoglobulin variable domain amino acid sequences of the anti-CD3 antibodies of the present technology include:

huOKT3
(SEQ ID NO: 7)
QVQLVQSGGGVVQPGRSLRLSCKASGYTFTRYTMHWVRQAPGKCLEWIG
YINPSRGYTNYNQKFKDRFTISRDNSKNTAFLQMDSLRPEDTGVYFCAR
YYDDHYSLDYWGQGTPVTVSS
huOKT3-DS
(SEQ ID NO: 8)
QVQLVQSGGGVVQPGRSLRLSCKASGYTFTRYTMHWVRQAPGKGLEWIG
YINPSRGYTNYNQKFKDRFTISRDNSKNTAFLQMDSLRPEDTGVYFCAR
YYDDHYSLDYWGQGTPVTVSS
VH-1 (humanness 85.7%)
(SEQ ID NO: 9)
QVQLQQSGAEVAKPGASVKMSCKASGYTFTRYTMHWVRQAPGQGLEWIG
YINPSRGYTNYAQKFQGRATLTTDKSISTAYMELSRLRSDDTAVYYCAR
YYDDHYSLDYWGQGTTLTVSS
VH-2 (humanness 85.7%)
(SEQ ID NO: 10)
QVQLQQSGAEVAKPGASVKVSCKASGYTFTRYTMHWVRQAPGQGLEWMG
YINPSRGYTNYNQKFKDRATLTRDKSISTAYMELSRLRSDDTAVYYCAR
YYDDHYSLDYWGQGTTLTVSS
VH-3 (humanness 85.7%)
(SEQ ID NO: 11)
QVQLVQSGAEVKKPGASVKMSCKASGYTFTRYTMHWVRQAPGQGLEWIG
YINPSRGYTNYNQKFKDRATMTTDKSISTAYMELSRLRSDDTAVYYCAR
YYDDHYSLDYWGQGTTLTVSS
VH-4 (humanness 85.7%)
(SEQ ID NO: 12)
QVQLQQSGAEVKKPGASVKMSCKASGYTFTRYTMHWVRQAPGQGLEWIG
YINPSRGYTNYNQKFKDRVTLTTDTSISTAYMELSRLRSDDTAVYYCAR
YYDDHYSLDYWGQGTTLTVSS
VH-1 H105 (humanness 85.7%)
(SEQ ID NO: 13)
QVQLQQSGAEVAKPGASVKMSCKASGYTFTRYTMHWVRQAPGQGLEWIG
YINPSRGYTNYAQKFQGRATLTTDKSISTAYMELSRLRSDDTAVYYCAR
YYDDHYSLDYWGCGTTLTVSS
VH-2 H105 (humanness 85.7%)
(SEQ ID NO: 14)
QVQLQQSGAEVAKPGASVKVSCKASGYTFTRYTMHWVRQAPGQGLEWMG
YINPSRGYTNYNQKFKDRATLTRDKSISTAYMELSRLRSDDTAVYYCAR
YYDDHYSLDYWGCGTTLTVSS
VH-3 H105 (humanness 85.7%)
(SEQ ID NO: 15)
QVQLVQSGAEVKKPGASVKMSCKASGYTFTRYTMHWVRQAPGQGLEWIG
YINPSRGYTNYNQKFKDRATMTTDKSISTAYMELSRLRSDDTAVYYCAR
YYDDHYSLDYWGCGTTLTVSS
VH-4 H105 (humanness 85.7%)
(SEQ ID NO: 16)
QVQLQQSGAEVKKPGASVKMSCKASGYTFTRYTMHWVRQAPGQGLEWIG
YINPSRGYTNYNQKFKDRVTLTTDTSISTAYMELSRLRSDDTAVYYCAR
YYDDHYSLDYWGCGTTLTVSS
VH-1 H44 (humanness 85.7%)
(SEQ ID NO: 17)
QVQLQQSGAEVAKPGASVKMSCKASGYTFTRYTMHWVRQAPGQCLEWIG
YINPSRGYTNYAQKFQGRATLTTDKSISTAYMELSRLRSDDTAVYYCAR
YYDDHYSLDYWGQGTTLTVSS
VH-2 H44 (humanness 85.7%)
(SEQ ID NO: 18)
QVQLQQSGAEVAKPGASVKVSCKASGYTFTRYTMHWVRQAPGQCLEWMG
YINPSRGYTNYNQKFKDRATLTRDKSISTAYMELSRLRSDDTAVYYCAR
YYDDHYSLDYWGQGTTLTVSS
VH-3 H44 (humanness 85.7%)
(SEQ ID NO: 19)
QVQLVQSGAEVKKPGASVKMSCKASGYTFTRYTMHWVRQAPGQCLEWIG
YINPSRGYTNYNQKFKDRATMTTDKSISTAYMELSRLRSDDTAVYYCAR
YYDDHYSLDYWGQGTTLTVSS
VH-4 H44 (humanness 85.7%)
(SEQ ID NO: 20)
QVQLQQSGAEVKKPGASVKMSCKASGYTFTRYTMHWVRQAPGQCLEWIG
YINPSRGYTNYNQKFKDRVTLTTDTSISTAYMELSRLRSDDTAVYYCAR
YYDDHYSLDYWGQGTTLTVSS
VH-1 H100B (humanness 85.7%)
(SEQ ID NO: 21)
QVQLQQSGAEVAKPGASVKMSCKASGYTFTRYTMHWVRQAPGQGLEWIG
YINPSRGYTNYAQKFQGRATLTTDKSISTAYMELSRLRSDDTAVYYCAR
YYDDHYSCDYWGQGTTLTVSS
VH-2 H100B (humanness 85.7%)
(SEQ ID NO: 22)
QVQLQQSGAEVAKPGASVKVSCKASGYTFTRYTMHWVRQAPGQGLEWMG
YINPSRGYTNYNQKFKDRATLTRDKSISTAYMELSRLRSDDTAVYYCAR
YYDDHYSCDYWGQGTTLTVSS
VH-3 H100B (humanness 85.7%)
(SEQ ID NO: 23)
QVQLVQSGAEVKKPGASVKMSCKASGYTFTRYTMHWVRQAPGQGLEWIG
YINPSRGYTNYNQKFKDRATMTTDKSISTAYMELSRLRSDDTAVYYCAR
YYDDHYSCDYWGQGTTLTVSS
VH-4 H100B (humanness 85.7%)
(SEQ ID NO: 24)
QVQLQQSGAEVKKPGASVKMSCKASGYTFTRYTMHWVRQAPGQGLEWIG
YINPSRGYTNYNQKFKDRVTLTTDTSISTAYMELSRLRSDDTAVYYCAR
YYDDHYSCDYWGQGTTLTVSS
VH-1 H100 (humanness 85.7%)
(SEQ ID NO: 25)
QVQLQQSGAEVAKPGASVKMSCKASGYTFTRYTMHWVRQAPGQGLEWIG
YINPSRGYTNYAQKFQGRATLTTDKSISTAYMELSRLRSDDTAVYYCAR
YYDDHCSLDYWGQGTTLTVSS
VH-2 H100 (humanness 85.7%)
(SEQ ID NO: 26)
QVQLQQSGAEVAKPGASVKVSCKASGYTFTRYTMHWVRQAPGQGLEWMG
YINPSRGYTNYNQKFKDRATLTRDKSISTAYMELSRLRSDDTAVYYCAR
YYDDHCSLDYWGQGTTLTVSS
VH-3 H100 (humanness 85.7%)
(SEQ ID NO: 27)
QVQLVQSGAEVKKPGASVKMSCKASGYTFTRYTMHWVRQAPGQGLEWIG
YINPSRGYTNYNQKFKDRATMTTDKSISTAYMELSRLRSDDTAVYYCAR
YYDDHCSLDYWGQGTTLTVSS
VH-4 H100 (humanness 85.7%)
(SEQ ID NO: 28)
QVQLQQSGAEVKKPGASVKMSCKASGYTFTRYTMHWVRQAPGQGLEWIG
YINPSRGYTNYNQKFKDRVTLTTDTSISTAYMELSRLRSDDTAVYYCAR
YYDDHCSLDYWGQGTTLTVSS
VH-1 H101 (humanness 85.7%)
(SEQ ID NO: 29)
QVQLQQSGAEVAKPGASVKMSCKASGYTFTRYTMHWVRQAPGQGLEWIG
YINPSRGYTNYAQKFQGRATLTTDKSISTAYMELSRLRSDDTAVYYCAR
YYDDHYSLCYWGQGTTLTVSS
VH-2 H101 (humanness 85.7%)
(SEQ ID NO: 30)
QVQLQQSGAEVAKPGASVKVSCKASGYTFTRYTMHWVRQAPGQGLEWMG
YINPSRGYTNYNQKFKDRATLTRDKSISTAYMELSRLRSDDTAVYYCAR
YYDDHYSLCYWGQGTTLTVSS
VH-3 H101 (humanness 85.7%)
(SEQ ID NO: 31)
QVQLVQSGAEVKKPGASVKMSCKASGYTFTRYTMHWVRQAPGQGLEWIG
YINPSRGYTNYNQKFKDRATMTTDKSISTAYMELSRLRSDDTAVYYCAR
YYDDHYSLCYWGQGTTLTVSS
VH-4 H101 (humanness 85.7%)
(SEQ ID NO: 32)
QVQLQQSGAEVKKPGASVKMSCKASGYTFTRYTMHWVRQAPGQGLEWIG
YINPSRGYTNYNQKFKDRVTLTTDTSISTAYMELSRLRSDDTAVYYCAR
YYDDHYSLCYWGQGTTLTVSS
Exemplary light chain immunoglobulin variable
domain amino acid sequences of the anti-CD3
antibodies of the present technology include:
huOKT3
(SEQ ID NO: 33)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
CGTKLQITR
huOKT3-DS
(SEQ ID NO: 34)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITR
VL-1 (humanness 85.2%)
(SEQ ID NO: 35)
DIQMTQSPSSLSASVGDRVTMTCSASSSVSYMNWYQQKPGKAPKRLIYD
TSKLASGVPSRFSGSGSGTDFTLTISSLQPEDFATYYCQQWSSNPFTFG
SGTKLEINR
VL-2 (humanness 85.2%)
(SEQ ID NO: 36)
DIQMTQSPSSLSASVGDRVTMTCSASSSVSYMNWYQQKPGKAPKLLIYD
TSKLASGVPSRFSGSGSGTDFTLTISSMQPEDFATYYCQQWSSNPFTFG
SGTKLEINR
VL-3 (humanness 85.2%)
(SEQ ID NO: 37)
DIQLTQSPSSLSASVGDRVTITCSASSSVSYMNWYQQKPGKAPKLLIYD
TSKLASGVPSRFSGSGSGTDYTLTISSLQPEDFATYYCQQWSSNPFTFG
SGTKLEINR
VL-4 (humanness 85.2%)
(SEQ ID NO: 38)
DIQLTQSPSSLSASVGDRVTITCSASSSVSYMNWYQQKPGKAPKLLIYD
TSKLASGVPSRFSGSGSGTDFTLTISSMQPEDFATYYCQQWSSNPFTFG
SGTKLEINR
VL-5 (humanness 85.2%)
(SEQ ID NO: 39)
DIQMTQSPSSLSASVGDRVTITCSASSSVSYMNWYQQKPGKAPKRWIYD
TSKLASGVPSRFSGSGSGTDFTLTISSLQPEDFATYYCQQWSSNPFTFG
SGTKLEINR
VL-6 (humanness 85.2%)
(SEQ ID NO: 40)
DIQMTQSPSSLSASVGDRVTITCSASSSVSYMNWYQQKPGKAPKLWIYD
TSKLASGVPSRFSGSGSGTDYTLTISSLQPEDFATYYCQQWSSNPFTFG
SGTKLEINR
VL-1 L100 (humanness 85.2%)
(SEQ ID NO: 41)
DIQMTQSPSSLSASVGDRVTMTCSASSSVSYMNWYQQKPGKAPKRLIYD
TSKLASGVPSRFSGSGSGTDFTLTISSLQPEDFATYYCQQWSSNPFTFG
CGTKLEINR
VL-2 L100 (humanness 85.2%)
(SEQ ID NO: 42)
DIQMTQSPSSLSASVGDRVTMTCSASSSVSYMNWYQQKPGKAPKLLIYD
TSKLASGVPSRFSGSGSGTDFTLTISSMQPEDFATYYCQQWSSNPFTFG
CGTKLEINR
VL-3 L100 (humanness 85.2%)
(SEQ ID NO: 43)
DIQLTQSPSSLSASVGDRVTITCSASSSVSYMNWYQQKPGKAPKLLIYD
TSKLASGVPSRFSGSGSGTDYTLTISSLQPEDFATYYCQQWSSNPFTFG
CGTKLEINR
VL-4 L100 (humanness 85.2%)
(SEQ ID NO: 44)
DIQLTQSPSSLSASVGDRVTITCSASSSVSYMNWYQQKPGKAPKLLIYD
TSKLASGVPSRFSGSGSGTDFTLTISSMQPEDFATYYCQQWSSNPFTFG
CGTKLEINR
VL-5 L100 (humanness 85.2%)
(SEQ ID NO: 45)
DIQMTQSPSSLSASVGDRVTITCSASSSVSYMNWYQQKPGKAPKRWIYD
TSKLASGVPSRFSGSGSGTDFTLTISSLQPEDFATYYCQQWSSNPFTFG
CGTKLEINR
VL-6 L100 (humanness 85.2%)
(SEQ ID NO: 46)
DIQMTQSPSSLSASVGDRVTITCSASSSVSYMNWYQQKPGKAPKLWIYD
TSKLASGVPSRFSGSGSGTDYTLTISSLQPEDFATYYCQQWSSNPFTFG
CGTKLEINR
VL-1 L43 (humanness 85.2%)
(SEQ ID NO: 47)
DIQMTQSPSSLSASVGDRVTMTCSASSSVSYMNWYQQKPGKCPKRLIYD
TSKLASGVPSRFSGSGSGTDFTLTISSLQPEDFATYYCQQWSSNPFTFG
SGTKLEINR
VL-2 L43 (humanness 85.2%)
(SEQ ID NO: 48)
DIQMTQSPSSLSASVGDRVTMTCSASSSVSYMNWYQQKPGKCPKLLIYD
TSKLASGVPSRFSGSGSGTDFTLTISSMQPEDFATYYCQQWSSNPFTFG
SGTKLEINR
VL-3 L43 (humanness 85.2%)
(SEQ ID NO: 49)
DIQLTQSPSSLSASVGDRVTITCSASSSVSYMNWYQQKPGKCPKLLIYD
TSKLASGVPSRFSGSGSGTDYTLTISSLQPEDFATYYCQQWSSNPFTFG
SGTKLEINR
VL-4 L43 (humanness 85.2%)
(SEQ ID NO: 50)
DIQLTQSPSSLSASVGDRVTITCSASSSVSYMNWYQQKPGKCPKLLIYD
TSKLASGVPSRFSGSGSGTDFTLTISSMQPEDFATYYCQQWSSNPFTFG
SGTKLEINR
VL-5 L43 (humanness 85.2%)
(SEQ ID NO: 51)
DIQMTQSPSSLSASVGDRVTITCSASSSVSYMNWYQQKPGKCPKRWIYD
TSKLASGVPSRFSGSGSGTDFTLTISSLQPEDFATYYCQQWSSNPFTFG
SGTKLEINR
VL-6 L43 (humanness 85.2%)
(SEQ ID NO: 52)
DIQMTQSPSSLSASVGDRVTITCSASSSVSYMNWYQQKPGKCPKLWIYD
TSKLASGVPSRFSGSGSGTDYTLTISSLQPEDFATYYCQQWSSNPFTFG
SGTKLEINR
VL-1 L49 (humanness 85.2%)
(SEQ ID NO: 53)
DIQMTQSPSSLSASVGDRVTMTCSASSSVSYMNWYQQKPGKAPKRLICD
TSKLASGVPSRFSGSGSGTDFTLTISSLQPEDFATYYCQQWSSNPFTFG
SGTKLEINR
VL-2 L49 (humanness 85.2%)
(SEQ ID NO: 54)
DIQMTQSPSSLSASVGDRVTMTCSASSSVSYMNWYQQKPGKAPKLLICD
TSKLASGVPSRFSGSGSGTDFTLTISSMQPEDFATYYCQQWSSNPFTFG
SGTKLEINR
VL-3 L49 (humanness 85.2%)
(SEQ ID NO: 55)
DIQLTQSPSSLSASVGDRVTITCSASSSVSYMNWYQQKPGKAPKLLICD
TSKLASGVPSRFSGSGSGTDYTLTISSLQPEDFATYYCQQWSSNPFTFG
SGTKLEINR
VL-4 L49 (humanness 85.2%)
(SEQ ID NO: 56)
DIQLTQSPSSLSASVGDRVTITCSASSSVSYMNWYQQKPGKAPKLLICD
TSKLASGVPSRFSGSGSGTDFTLTISSMQPEDFATYYCQQWSSNPFTFG
SGTKLEINR
VL-5 L49 (humanness 85.2%)
(SEQ ID NO: 57)
DIQMTQSPSSLSASVGDRVTITCSASSSVSYMNWYQQKPGKAPKRWICD
TSKLASGVPSRFSGSGSGTDFTLTISSLQPEDFATYYCQQWSSNPFTFG
SGTKLEINR
VL-6 L49 (humanness 85.2%)
(SEQ ID NO: 58)
DIQMTQSPSSLSASVGDRVTITCSASSSVSYMNWYQQKPGKAPKLWICD
TSKLASGVPSRFSGSGSGTDYTLTISSLQPEDFATYYCQQWSSNPFTFG
SGTKLEINR
VL-1 L50 (humanness 85.2%)
(SEQ ID NO: 59)
DIQMTQSPSSLSASVGDRVTMTCSASSSVSYMNWYQQKPGKAPKRLIYC
TSKLASGVPSRFSGSGSGTDFTLTISSLQPEDFATYYCQQWSSNPFTFG
SGTKLEINR
VL-2 L50 (humanness 85.2%)
(SEQ ID NO: 60)
DIQMTQSPSSLSASVGDRVTMTCSASSSVSYMNWYQQKPGKAPKLLIYC
TSKLASGVPSRFSGSGSGTDFTLTISSMQPEDFATYYCQQWSSNPFTFG
SGTKLEINR
VL-3 L50 (humanness 85.2%)
(SEQ ID NO: 61)
DIQLTQSPSSLSASVGDRVTITCSASSSVSYMNWYQQKPGKAPKLLIYC
TSKLASGVPSRFSGSGSGTDYTLTISSLQPEDFATYYCQQWSSNPFTFG
SGTKLEINR
VL-4 L50 (humanness 85.2%)
(SEQ ID NO: 62)
DIQLTQSPSSLSASVGDRVTITCSASSSVSYMNWYQQKPGKAPKLLIYC
TSKLASGVPSRFSGSGSGTDFTLTISSMQPEDFATYYCQQWSSNPFTFG
SGTKLEINR
VL-5 L50 (humanness 85.2%)
(SEQ ID NO: 63)
DIQMTQSPSSLSASVGDRVTITCSASSSVSYMNWYQQKPGKAPKRWIYC
TSKLASGVPSRFSGSGSGTDFTLTISSLQPEDFATYYCQQWSSNPFTFG
SGTKLEINR
VL-6 L50 (humanness 85.2%)
(SEQ ID NO: 64)
DIQMTQSPSSLSASVGDRVTITCSASSSVSYMNWYQQKPGKAPKLWIYC
TSKLASGVPSRFSGSGSGTDYTLTISSLQPEDFATYYCQQWSSNPFTFG
SGTKLEINR
VL-1 L46 (humanness 85.2%)
(SEQ ID NO: 65)
DIQMTQSPSSLSASVGDRVTMTCSASSSVSYMNWYQQKPGKAPKCLIYD
TSKLASGVPSRFSGSGSGTDFTLTISSLQPEDFATYYCQQWSSNPFTFG
SGTKLEINR
VL-2 L46 (humanness 85.2%)
(SEQ ID NO: 66)
DIQMTQSPSSLSASVGDRVTMTCSASSSVSYMNWYQQKPGKAPKCLIYD
TSKLASGVPSRFSGSGSGTDFTLTISSMQPEDFATYYCQQWSSNPFTFG
SGTKLEINR
VL-3 L46 (humanness 85.2%)
(SEQ ID NO: 67)
DIQLTQSPSSLSASVGDRVTITCSASSSVSYMNWYQQKPGKAPKCLIYD
TSKLASGVPSRFSGSGSGTDYTLTISSLQPEDFATYYCQQWSSNPFTFG
SGTKLEINR
VL-4 L46 (humanness 85.2%)
(SEQ ID NO: 68)
DIQLTQSPSSLSASVGDRVTITCSASSSVSYMNWYQQKPGKAPKCLIYD
TSKLASGVPSRFSGSGSGTDFTLTISSMQPEDFATYYCQQWSSNPFTFG
SGTKLEINR
VL-5 L46 (humanness 85.2%)
(SEQ ID NO: 69)
DIQMTQSPSSLSASVGDRVTITCSASSSVSYMNWYQQKPGKAPKCWIYD
TSKLASGVPSRFSGSGSGTDFTLTISSLQPEDFATYYCQQWSSNPFTFG
SGTKLEINR
VL-6 L46 (humanness 85.2%)
(SEQ ID NO: 70)
DIQMTQSPSSLSASVGDRVTITCSASSSVSYMNWYQQKPGKAPKCWIYD
TSKLASGVPSRFSGSGSGTDYTLTISSLQPEDFATYYCQQWSSNPFTFG
SGTKLEINR

Additionally or alternatively, in some embodiments, the anti-CD3 multi-specific antibodies of the present technology include a CD3 binding domain comprising a heavy chain immunoglobulin variable domain (V_{H}) and a light chain immunoglobulin variable domain (V_{L}), wherein: (a) the V_{H} comprises an amino acid sequence selected from any one of SEQ ID NOs: 7-32; and/or (b) the V_{L} comprises an amino acid sequence selected from any one of SEQ ID NOs: 33-70. Additionally or alternatively, in some embodiments, the anti-CD3 multi-specific antibodies may be used to arm the EATs of the present technology.

In certain embodiments, the anti-CD3 multi-specific antibodies of the present technology includes one or more of the following characteristics: (a) a light chain immunoglobulin variable domain sequence that is at least 80%, at least 85%, at least 90%, at least 95%, or at least 99% identical to the light chain immunoglobulin variable domain sequence of any one of SEQ ID NOs: 33-70; and/or (b) a heavy chain immunoglobulin variable domain sequence that is at least 80%, at least 85%, at least 90%, at least 95%, or at least 99% identical to the heavy chain immunoglobulin variable domain sequence of any one of SEQ ID NOs: 7-32. In another aspect, one or more amino acid residues in the immunoglobulin-related compositions provided herein are substituted with another amino acid. The substitution may be a “conservative substitution” as defined herein. Additionally or alternatively, in some embodiments, the anti-CD3 multi-specific antibodies may be used to arm the EATs of the present technology.

In some embodiments, the anti-CD3 multi-specific antibodies of the present technology bind to the extracellular domain of a CD3 polypeptide. In certain embodiments, the epitope is a conformational epitope or non-conformational epitope. In some embodiments, the CD3 polypeptide has the amino acid sequence of SEQ ID NO: 71. Additionally or alternatively, in some embodiments, the anti-CD3 multi-specific antibodies may be used to arm the EATs of the present technology.

NCBI Ref: NP_000724.1 Homo sapiens T cell surface glycoprotein CD3 epsilon chain precursor (SEQ ID NO: 71)

MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

Additionally or alternatively, in some embodiments, the anti-CD3 multi-specific antibodies bind to the extracellular domain of a CD3 polypeptide. In certain embodiments, the extracellular domain comprises a CD3ε subunit including a linear stretch of sequence on the F-G loop. In some embodiments, the CD3ε subunit may comprise three discontinuous regions: residues 79ε-85ε (the F-G loop), residue 34ε (the first residue of the ßC strand), and residues 46ε and 48ε (the C′-D loop).

In any of the above embodiments, the anti-CD3 multi-specific antibodies further comprises a Fc domain of any isotype, e.g., but are not limited to, IgG (including IgG1, IgG2, IgG3, and IgG4).

Non-limiting examples of constant region sequences include:

Human IgG1 constant region, Uniprot: P01857
(SEQ ID NO: 72)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human IgG2 constant region, Uniprot: P01859
(SEQ ID NO: 73)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPGK
Human IgG3 constant region, Uniprot: P01860
(SEQ ID NO: 74)
ASTKGPSVFPLAPCSRSTSGGTAALGCLVKDYFPEPVTVSWNSGALTSG
VHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEP
KSCDTPPPCPRCPAPELLGGPSVFLFPPKPKDTLMISRTPEVTCVVVDV
SHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVS
LTCLVKGFYPSDIAVEWESSGQPENNYNTTPPMLDSDGSFFLYSKLTVD
KSRWQQGNIFSCSVMHEALHNRFTQKSLSLSPGK
Human IgG4 constant region, Uniprot: P01861
(SEQ ID NO: 75)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
Human Ig kappa constant region, Uniprot: P01834
(SEQ ID NO: 76)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

In some embodiments, the anti-CD3 multi-specific antibodies of the present technology comprise a heavy chain constant region that is at least 80%, at least 85%, at least 90%, at least 95%, at least 99%, or is 100% identical to SEQ ID NOs: 72-75. Additionally or alternatively, in some embodiments, the anti-CD3 multi-specific antibodies may be used to arm the EATs of the present technology.

Additionally or alternatively, in some embodiments, the anti-CD3 multi-specific antibodies of the present technology comprise a light chain constant region that is at least 80%, at least 85%, at least 90%, at least 95%, at least 99%, or is 100% identical to SEQ ID NO: 76. Additionally or alternatively, in some embodiments, the anti-CD3 multi-specific antibodies may be used to arm the EATs of the present technology.

In certain embodiments, the anti-CD3 multi-specific antibodies of the present technology contain an IgG1 constant region comprising one or more amino acid substitutions selected from the group consisting of N297A and K322A. Additionally or alternatively, in some embodiments, the immunoglobulin-related compositions contain an IgG4 constant region comprising a S228P mutation. Additionally or alternatively, in some embodiments, the anti-CD3 multi-specific antibodies may be used to arm the EATs of the present technology.

Additionally or alternatively, in some embodiments, the anti-CD3 multi-specific antibodies comprises a DOTA binding domain. The DOTA binding domain may include a V_{H} having the amino acid sequence of SEQ ID NO: 77 and/or a V_{L} having the amino acid sequence of SEQ ID NO: 78.

(SEQ ID NO: 77)
HVQLVESGGGLVQPGGSLRLSCAASGFSLTDYGVHWVRQAPGKGLEWL
GVIWSGGGTAYNTALISRFTISRDNSKNTLYLQMNSLRAEDTAVYYCAR
RGSYPYNYFDAWGCGTLVTVSS
(SEQ ID NO: 78)
QAVVTQEPSLTVSPGGTVTLTCGSSTGAVTASNYANWVQQKPGQCPRGL
IGGHNNRPPGVPARFSGSLLGGKAALTLLGAQPEDEAEYYCALWYSDHW
VIGGGTKLTVLG

In certain embodiments, the DOTA binding domain is a scFv and/or may comprise an amino acid sequence selected from the group consisting of:

(SEQ ID NO: 79)
HVQLVESGGGLVQPGGSLRLSCAASGFSLTDYGVHWVRQAPGKGLEWLG
VIWSGGGTAYNTALISRFTISRDNSKNTLYLQMNSLRAEDTAVYYCARR
GSYPYNYFDAWGCGTLVTVSSGGGGSGGGGSGGGGSQAVVTQEPSLTVS
PGGTVTLTCGSSTGAVTASNYANWVQQKPGQCPRGLIGGHNNRPPGVPA
RFSGSLLGGKAALTLLGAQPEDEAEYYCALWYSDHWVIGGGTKLTVLG;
and
(SEQ ID NO: 80)
HVQLVESGGGLVQPGGSLRLSCAASGFSLTDYGVHWVRQAPGKGLEWLG
VIWSGGGTAYNTALISRFTISRDNSKNTLYLQMNSLRAEDTAVYYCARR
GSYPYNYFDAWGCGTLVTVSSGGGGSGGGGSGGGGSGGGGSGGGGSGGG
GSQAVVTQEPSLTVSPGGTVTLTCGSSTGAVTASNYANWVQQKPGQCPR
GLIGGHNNRPPGVPARFSGSLLGGKAALTLLGAQPEDEAEYYCALWYSD
HWVIGGGTKLTVLG.
*(G4S)n linker sequence (SEQ ID NO: 155) is shown in boldface

Additionally or alternatively, in some embodiments, the anti-CD3 multi-specific antibody comprises a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, or a variant thereof having one or more conservative amino acid substitutions. Additionally or alternatively, in some embodiments, the anti-CD3 multi-specific antibody comprises a light chain (LC) amino acid sequence comprising SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, or a variant thereof having one or more conservative amino acid substitutions.

In other embodiments, the anti-CD3 multi-specific antibody comprises (a) a LC sequence that is at least 80%, at least 85%, at least 90%, at least 95%, or at least 99% identical to the LC sequence present in SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, or SEQ ID NO: 149; and/or (b) a HC sequence that is at least 80%, at least 85%, at least 90%, at least 95%, or at least 99% identical to the HC sequence present in SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, or SEQ ID NO: 150.

Additionally or alternatively, in some embodiments, the anti-CD3 multi-specific antibody comprises a HC amino acid sequence and a LC amino acid sequence selected from the group consisting of: SEQ ID NO: 82 and SEQ ID NO: 81, SEQ ID NO: 84 and SEQ ID NO: 83, SEQ ID NO: 86 and SEQ ID NO: 85, SEQ ID NO: 88 and SEQ ID NO: 87, SEQ ID NO: 90 and SEQ ID NO: 89, SEQ ID NO: 92 and SEQ ID NO: 91, SEQ ID NO: 94 and SEQ ID NO: 93, SEQ ID NO: 96 and SEQ ID NO: 95, SEQ ID NO: 98 and SEQ ID NO: 97, SEQ ID NO: 100 and SEQ ID NO: 99, SEQ ID NO: 102 and SEQ ID NO: 101, SEQ ID NO: 104 and SEQ ID NO: 103, SEQ ID NO: 106 and SEQ ID NO: 105, SEQ ID NO: 108 and SEQ ID NO: 107, SEQ ID NO: 110 and SEQ ID NO: 109, SEQ ID NO: 112 and SEQ ID NO: 111, SEQ ID NO: 114 and SEQ ID NO: 113, SEQ ID NO: 116 and SEQ ID NO: 115, SEQ ID NO: 118 and SEQ ID NO: 117, SEQ ID NO: 120 and SEQ ID NO: 119, SEQ ID NO: 122 and SEQ ID NO: 121, SEQ ID NO: 124 and SEQ ID NO: 123, SEQ ID NO: 126 and SEQ ID NO: 125, SEQ ID NO: 128 and SEQ ID NO: 127, SEQ ID NO: 130 and SEQ ID NO: 129, SEQ ID NO: 132 and SEQ ID NO: 131, SEQ ID NO: 134 and SEQ ID NO: 133, SEQ ID NO: 136 and SEQ ID NO: 135, SEQ ID NO: 138 and SEQ ID NO: 137, SEQ ID NO: 140 and SEQ ID NO: 139, SEQ ID NO: 142 and SEQ ID NO: 141, SEQ ID NO: 144 and SEQ ID NO: 143, SEQ ID NO: 146 and SEQ ID NO: 145, SEQ ID NO: 148 and SEQ ID NO: 147, and SEQ ID NO: 150 and SEQ ID NO: 149, respectively.

Additionally or alternatively, in some embodiments, the anti-CD3 multi-specific antibody comprise a first LC amino acid sequence, a first HC amino acid sequence, a second LC amino acid sequence, and a second HC amino acid sequence selected from the group consisting of SEQ ID NO: 99, SEQ ID NO: 100, SEQ ID NO: 101, and SEQ ID NO: 102; SEQ ID NO: 103, SEQ ID NO: 104, SEQ ID NO: 105, and SEQ ID NO: 106; SEQ ID NO: 107, SEQ ID NO: 108, SEQ ID NO: 109, and SEQ ID NO: 110; SEQ ID NO: 111, SEQ ID NO: 112, SEQ ID NO: 113, and SEQ ID NO: 114; SEQ ID NO: 115, SEQ ID NO: 116, SEQ ID NO: 117, and SEQ ID NO: 118; SEQ ID NO: 119, SEQ ID NO: 120, SEQ ID NO: 121, and SEQ ID NO: 122; SEQ ID NO: 123, SEQ ID NO: 124, SEQ ID NO: 125, and SEQ ID NO: 126; SEQ ID NO: 127, SEQ ID NO: 128, SEQ ID NO: 129, and SEQ ID NO: 130; SEQ ID NO: 131, SEQ ID NO: 132, SEQ ID NO: 133, and SEQ ID NO: 134; SEQ ID NO: 135, SEQ ID NO: 136, SEQ ID NO: 137, and SEQ ID NO: 138; SEQ ID NO: 139, SEQ ID NO: 140, SEQ ID NO: 141, and SEQ ID NO: 142; SEQ ID NO: 143, SEQ ID NO: 144, SEQ ID NO: 145, and SEQ ID NO: 146; and SEQ ID NO: 147, SEQ ID NO: 148, SEQ ID NO: 149, and SEQ ID NO: 150; respectively.

Additionally or alternatively, in some embodiments, the anti-CD3 multi-specific antibodies of the present disclosure bind one or more additional target antigens selected from the group consisting of CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), or a small molecule DOTA-based hapten. Additionally or alternatively, in some embodiments, the anti-CD3 multi-specific antibodies may be used to arm the EATs of the present technology.

In some aspects, the anti-CD3 multi-specific antibodies described herein contain structural modifications to facilitate rapid binding and cell uptake and/or slow release. In some aspects, the anti-CD3 multi-specific antibodies of the present technology (e.g., an antibody) may contain a deletion in the CH2 constant heavy chain region to facilitate rapid binding and cell uptake and/or slow release. Additionally or alternatively, in some embodiments, the anti-CD3 multi-specific antibodies may be used to arm the EATs of the present technology.

In any of the above embodiments of the anti-CD3 multi-specific antibodies or the EATs of the present technology, the anti-CD3 multi-specific antibodies may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In some embodiments, the anti-CD3 multi-specific antibodies or EATs of the present technology bind specifically to at least one CD3 polypeptide. In some embodiments, the anti-CD3 multi-specific antibodies or EATs of the present technology bind at least one CD3 polypeptide with a dissociation constant (K_{D}) of about 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, the antibodies comprise a human antibody framework region.

Formulations Including Dexamethasone, and/or the T Cell Engaging Multi-specific Antibodies of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with dexamethasone and/or a T cell engaging multi-specific antibody may be employed. Suitable methods include in vitro, ex vivo, or in vivo methods. In vivo methods typically include the administration of dexamethasone and/or a T cell engaging multi-specific antibody, such as those described herein, to a mammal, suitably a human. When used in vivo for therapy, the dexamethasone and/or T cell engaging multi-specific antibody are administered to the subject in effective amounts (i.e., amounts that have desired therapeutic effect). The dose and dosage regimen will depend upon the degree of the disease symptoms in the subject, the characteristics of the dexamethasone and/or the T cell engaging multi-specific antibody, e.g., its therapeutic index, the subject, and the subject's history.

The effective amount may be determined during pre-clinical trials and clinical trials by methods familiar to physicians and clinicians. An effective amount of the dexamethasone and/or the T cell engaging multi-specific antibody useful in the methods may be administered to a mammal in need thereof by any of a number of well-known methods for administering pharmaceutical compounds. The dexamethasone and/or the T cell engaging multi-specific antibody may be administered systemically or locally.

The dexamethasone and/or the T cell engaging multi-specific antibody can be incorporated into pharmaceutical compositions for administration, singly or in combination, to a subject for the treatment or prevention of a disorder described herein. Such compositions typically include the active agent and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

In some embodiments, the dexamethasone or the T cell engaging multi-specific antibody described herein is administered by a parenteral route or a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The dexamethasone and/or the T cell engaging multi-specific antibody described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, compositions including the dexamethasone and/or the T cell engaging multi-specific antibody of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of the dexamethasone and/or the T cell engaging multi-specific antibody of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

The dexamethasone and/or the T cell engaging multi-specific antibody of the present technology can be formulated in a carrier system. The carrier can be a colloidal system. The colloidal system can be a liposome, a phospholipid bilayer vehicle. In one embodiment, the therapeutic dexamethasone and/or T cell engaging multi-specific antibody is encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the dexamethasone and/or the T cell engaging multi-specific antibody can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the dexamethasone and/or the T cell engaging multi-specific antibody are prepared with carriers that will protect the dexamethasone and/or the T cell engaging multi-specific antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The dexamethasone and/or the T cell engaging multi-specific antibody can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of dexamethasone and/or a T cell engaging multi-specific antibody can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the dexamethasone and/or the T cell engaging multi-specific antibody exhibit high therapeutic indices. While the dexamethasone and/or the T cell engaging multi-specific antibody that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For the dexamethasone and/or the T cell engaging multi-specific antibody, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the dexamethasone and/or a T cell engaging multi-specific antibody, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the dexamethasone and/or the T cell engaging multi-specific antibody ranges from 0.001-10,000 micrograms per kg body weight. In one embodiment, the dexamethasone and/or the T cell engaging multi-specific antibody concentrations is in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of the dexamethasone and/or the T cell engaging multi-specific antibody may be defined as a concentration of dexamethasone and/or a T cell engaging multi-specific antibody at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the dexamethasone and/or the T cell engaging multi-specific antibody of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### EATs of the Present Technology

The present disclosure provides ex vivo armed T cells (EATs) that are coated or complexed with an effective arming dose of multi-specific (e.g., bispecific) antibodies that bind to CD3 and at least one additional target antigen (e.g., antigen that is expressed by tumor cells and/or a DOTA-based hapten). The EATs of the present disclosure may be armed with an effective arming dose of at least one type of anti-CD3 multi-specific antibody described herein. In certain embodiments, the EATs of the present disclosure may be armed with an effective arming dose of at least 1, 2, 3, 4, 5, 6, 7, 8, 9, 10 or more types of anti-CD3 multi-specific antibodies described herein.

T cells are lymphocytes that mature in the thymus and are chiefly responsible for cell-mediated immunity. T cells are involved in the adaptive immune system. The T cells included in the EATs of the presently disclosed subject matter can be any type of T cells, including, but not limited to, T helper cells, cytotoxic T cells, memory T cells (including central memory T cells), stem-cell-like memory T cells (or stem-like memory T cells), and two types of effector memory T cells: e.g., T_{EM} cells and TEMRA cells, Regulatory T cells (also known as suppressor T cells), Natural killer T cells, Mucosal associated invariant T (MAIT) cells, EBV-specific cytotoxic T cells (EBV-CTLs), αβ T cells and γδ T cells. Cytotoxic T cells (CTL or killer T cells) are a subset of T lymphocytes capable of inducing the death of infected somatic or tumor cells. In some embodiments, the T cells further comprise a chimeric antigen receptor (CAR).

In any and all embodiments of the EATs disclosed herein, the at least one type of anti-CD3 multi-specific antibody exhibits surface densities between about 500 to about 20,000 molecules per T cell or between about 1,500 to 10,000 molecules per T cell. In certain embodiments, the at least one type of anti-CD3 multi-specific antibody exhibits surface densities of about 500, about 550, about 600, about 650, about 700, about 750, about 800, about 850, about 900, about 950, about 1000, about 1250, about 1500, about 1750, about 2000, about 2250, about 2500, about 2750, about 3000, about 3250, about 3500, about 3750, about 4000, about 4250, about 4500, about 4750, about 5000, about 5500, about 6000, about 6500, about 7000, about 7500, about 8000, about 8500, about 9000, about 9500, about 10,000, about 11,000, about 12,000, about 13,000, about 14,000, about 15,000, about 16,000, about 17,000, about 18,000, about 19,000, about 20,000, about 25,000, about 30,000, or about 35,000 molecules per T cell. Values and ranges intermediate to the recited values are also contemplated.

In any and all embodiments of the EATs disclosed herein, T cells are armed ex vivo with the at least one type of anti-CD3 multi-specific antibody at doses (e.g., effective arming dose) ranging between about 0.05 μg/10⁶ T cells to about 5 μg/10⁶ T cells. In certain embodiments, T cells are armed ex vivo with the at least one type of anti-CD3 multi-specific antibody at a dose (e.g., effective arming dose) of about 0.05 μg/10⁶ T cells, about 0.06 μg/10⁶ T cells, about 0.07 μg/10⁶ T cells, about 0.08 μg/10⁶ T cells, about 0.09 μg/10⁶ T cells, about 0.1 μg/10⁶ T cells, about 0.2 μg/10⁶ T cells, about 0.3 μg/10⁶ T cells, about 0.4 μg/10⁶ T cells, about 0.5 μg/10⁶ T cells, about 0.6 μg/10⁶ T cells, about 0.7 μg/10⁶ T cells, about 0.8 μg/10⁶ T cells, about 0.9 μg/10⁶ T cells, about 1.0 μg/10⁶ T cells, about 1.5 μg/10⁶ T cells, about 2.0 μg/10⁶ T cells, about 2.5 μg/10⁶ T cells, about 3.0 μg/10⁶ T cells, about 3.5 μg/10⁶ T cells, about 4.0 μg/10⁶ T cells, about 4.5 μg/10⁶ T cells, or about 5.0 μg/10⁶ T cells. Values and ranges intermediate to the recited values are also contemplated. Additionally or alternatively, in some embodiments, T cells are armed ex vivo by contacting T cells with an effective arming dose of the at least one type of anti-CD3 multi-specific antibody for about 5-60 minutes at room temperature. In certain embodiments, T cells are armed ex vivo by contacting T cells with an effective arming dose of the at least one type of anti-CD3 multi-specific antibody for about 5 mins, about 10 mins, about 15 mins, about 20 mins, about 25 mins, about 30 mins, about 35 mins, about 40 mins, about 45 mins, about 50 mins, about 55 mins, or about 60 mins at room temperature. Values and ranges intermediate to the recited values are also contemplated.

Additionally or alternatively, in some embodiments, the EATs are freshly prepared or have been cryopreserved. In certain embodiments, the EATs are cryopreserved for a period of about 2 hours to about 1 or more years. In some embodiments, the EATs are cryopreserved for a period of at least 2 hours, at least 3 hours, at least 4 hours, at least 5 hours, at least 6 hours, at least 7 hours, at least 8 hours, at least 9 hours, at least 10 hours, at least 12 hours, at least 24 hours, at least 48 hours, at least 3 days, at least 4 days, at least 5 days, at least 6 days, at least 1 week, at least 2 weeks, at least 3 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, or at least 12 months. Values and ranges intermediate to the recited values are also contemplated.

The EATs can be generated using peripheral donor lymphocytes, e.g., those disclosed in Panelli et al., J Immunol 164:495-504 (2000); Panelli et al., J Immunol 164:4382-4392 (2000) (disclosing lymphocyte cultures derived from tumor infiltrating lymphocytes (TTLs) in tumor biopsies). The EATs can be autologous, non-autologous (e.g., allogeneic), or derived in vitro from lymphoid progenitor or stem cells.

The unpurified source of T cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cells initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

Administration. EATs of the presently disclosed subject matter can be provided systemically or directly to a subject for treating or preventing a neoplasia. In certain embodiments, EATs are directly injected into an organ of interest (e.g., an organ affected by a neoplasia). Alternatively or additionally, the EATs are provided indirectly to the organ of interest, for example, by administration into the circulatory system (e.g., the tumor vasculature) or into the solid tumor. Expansion and differentiation agents can be provided prior to, during or after administration of cells and compositions to promote maintenance/survival of T cells in vitro or in vivo.

EATs of the presently disclosed subject matter can be administered in any physiologically acceptable vehicle, systemically or regionally, normally intravascularly, intraperitoneally, intrathecally, or intrapleurally, although they may also be introduced into bone or other convenient site. In certain embodiments, at least 1×10⁵ cells, at least 1×10⁶ cells or 1×10¹⁰ or more cells can be administered. A cell population comprising EATs can comprise a purified population of cells. Those skilled in the art can readily determine the percentage of EATs in a cell population using various well-known methods, such as fluorescence activated cell sorting (FACS). The ranges of purity in cell populations comprising EATs can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The EATs can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising EATs coated or complexed with an effective arming dose of at least one type of anti-CD3 multi-specific antibody described herein and a pharmaceutically acceptable carrier. Administration can be autologous or non-autologous. For example, EATs coated or complexed with an effective arming dose of at least one type of anti-CD3 multi-specific antibody described herein and compositions comprising thereof can be obtained from one subject, and administered to the same subject or a different, compatible subject. Peripheral blood derived EATs of the presently disclosed subject matter can be administered via localized injection, including catheter administration, systemic injection, localized injection, intravenous injection, or parenteral administration. When administering a pharmaceutical composition of the presently disclosed subject matter (e.g., a pharmaceutical composition comprising EATs coated or complexed with an effective arming dose of at least one type of anti-CD3 multi-specific antibody described herein), it can be formulated in a unit dosage injectable form (solution, suspension, emulsion).

For treatment, the amount of the EATs provided herein administered is an amount effective in producing the desired effect, for example, treatment of a cancer or one or more symptoms of a cancer. An effective amount can be provided in one or a series of administrations of the EATs provided herein. An effective amount can be provided in a bolus or by continuous perfusion. For adoptive immunotherapy using EATs, cell doses in the range of about 10⁴ to about 10¹⁰ are typically infused.

The EATs of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the EATs and the compositions comprising thereof are intravenously administered to the subject in need. Methods for administering cells for adoptive cell therapies, including, for example, donor lymphocyte infusion and cellular immunotherapies, and regimens for administration are known in the art and can be employed for administration of the EATs provided herein.

Formulations. EATs coated or complexed with an effective arming dose of at least one type of anti-CD3 multi-specific antibody described herein and compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising EATs, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the EATs of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the EATs as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the EATs of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ EATs of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more EATs of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, EATs are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %.

Toxicity. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation. Optimally, an effective amount (e.g., dose) of an EAT described herein will provide therapeutic benefit without causing substantial toxicity to the subject. Toxicity of the EAT described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the EAT described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

### Combination Therapy of the Present Technology

The methods disclosed herein are effective in decreasing or delaying CRS and/or improving the efficacy of any of the anti-tumor T cell engaging (e.g., anti-CD3) multi-specific antibodies or EATs described herein in a subject in need thereof. For example, when administered to the appropriate subject as determined by the methods of the present technology, a therapeutically effective amount of dexamethasone and the anti-CD3 multi-specific antibody or EAT may partially or completely alleviate one or more symptoms of cancer and/or lead to increased survival, reduced tumor burden, reduced tumor relapse, reduction of the number of cancer cells, reduction of the tumor size, eradication of tumor, inhibition of cancer cell infiltration into peripheral organs, inhibition or stabilization of tumor growth, and stabilization or improvement of quality of life in the subject. The presently disclosed subject matter also provides methods of increasing or lengthening survival of a subject having a neoplasia (e.g., a tumor). In one non-limiting example, the method of increasing or lengthening survival of a subject having neoplasia (e.g., a tumor) comprises administering an effective amount of dexamethasone and an effective amount of an anti-CD3 multi-specific antibody or EAT of the present technology to the subject, thereby increasing or lengthening survival of the subject.

In one aspect, the present disclosure provides a method for treating cancer or inhibiting tumor growth in a subject in need thereof comprising administering to the subject an effective amount of dexamethasone and an effective amount of any and all embodiments of the anti-CD3 multi-specific antibody disclosed herein. In another aspect, the present disclosure provides a method for treating cancer or inhibiting tumor growth in a subject in need thereof comprising administering to the subject an effective amount of dexamethasone and an effective amount of any and all embodiments of the EATs disclosed herein.

In one aspect, the present disclosure provides a method for reducing or delaying cytokine release syndrome (CRS) in a subject in need thereof comprising administering to the subject an effective amount of dexamethasone and an effective amount of any and all embodiments of the anti-CD3 multi-specific antibody disclosed herein. In another aspect, the present disclosure provides a method for reducing or delaying cytokine release syndrome (CRS) in a subject in need thereof comprising administering to the subject an effective amount of dexamethasone and an effective amount of any and all embodiments of the EATs disclosed herein. Examples of immune-activating cytokines that cause CRS include granulocyte macrophage colony stimulating factor (GM-CSF), IFNα, IFN-β, IFN-γ, TNF-α, IL-2, IL-3, IL-6, IL-10, IL-11, IL-7, IL-12, IL-15, IL-21, interferon regulatory factor 7 (IRF7), and combinations thereof.

In any and all embodiments of the methods disclosed herein, dexamethasone may be administered at a high dosage, an intermediate dosage or a low dosage. In some embodiments of the methods disclosed herein, dexamethasone may be administered at one or more time points at a dose ranging from about 0.1 mg/kg to about 35 mg/kg. In certain embodiments, the dose of dexamethasone is about 0.1 mg/kg-about 0.15 mg/kg, about 0.16 mg/kg-about 0.2 mg/kg, about 0.2 mg/kg-about 0.25 mg/kg, about 0.26 mg/kg-about 0.3 mg/kg, about 0.3 mg/kg-about 0.35 mg/kg, about 0.36 mg/kg-about 0.4 mg/kg, about 0.4 mg/kg-about 0.45 mg/kg, about 0.46 mg/kg-about 0.5 mg/kg, about 0.5 mg/kg-about 0.55 mg/kg, about 0.56 mg/kg-about 0.6 mg/kg, about 0.6 mg/kg-about 0.65 mg/kg, about 0.66 mg/kg-about 0.7 mg/kg, about 0.7 mg/kg-about 0.75 mg/kg, about 0.76 mg/kg-about 0.8 mg/kg, about 0.8 mg/kg-about 0.85 mg/kg, about 0.86 mg/kg-about 0.9 mg/kg, about 0.9 mg/kg-about 0.95 mg/kg, about 0.96 mg/kg-about 1 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, or about 35 mg/kg. In some embodiments, the subject is human and dexamethasone may be administered at one or more time points at a dose ranging from about 0.16 mg/kg to about 8 mg/kg.

In one aspect, the present disclosure provides a method for treating cancer or ameliorating cytokine release syndrome in a subject in need thereof comprising (a) administering to the subject a first effective dose of dexamethasone, (b) administering to the subject a first effective amount of any and all embodiments of the anti-CD3 multi-specific antibody of the present technology about 1 hour after administration of the first effective dose of dexamethasone, (c) administering to the subject a second effective dose of dexamethasone about 72-96 hours after administration of the first effective dose of dexamethasone, (d) administering to the subject a second effective amount of the anti-CD3 multi-specific antibody about 1 hour after administration of the second effective dose of dexamethasone, and (e) repeating steps (a)-(d) for at least one additional cycle. In another aspect, the present disclosure provides a method for treating cancer or ameliorating cytokine release syndrome in a subject in need thereof comprising (a) administering to the subject a first effective dose of dexamethasone, (b) administering to the subject a first effective amount of any and all embodiments of the EATs disclosed herein about 1 hour after administration of the first effective dose of dexamethasone, (c) administering to the subject a second effective dose of dexamethasone about 72-96 hours after administration of the first effective dose of dexamethasone, (d) administering to the subject a second effective amount of the EATs about 1 hour after administration of the second effective dose of dexamethasone, and (e) repeating steps (a)-(d) for at least one additional cycle. The first second effective dose and second effective dose of dexamethasone may be identical or different. In some embodiments, the first and/or second effective dose of dexamethasone is about 0.1 mg/kg to about 35 mg/kg. In certain embodiments, the first and/or second effective dose of dexamethasone is about 0.1 mg/kg-about 0.15 mg/kg, about 0.16 mg/kg-about 0.2 mg/kg, about 0.2 mg/kg-about 0.25 mg/kg, about 0.26 mg/kg-about 0.3 mg/kg, about 0.3 mg/kg-about 0.35 mg/kg, about 0.36 mg/kg-about 0.4 mg/kg, about 0.4 mg/kg-about 0.45 mg/kg, about 0.46 mg/kg-about 0.5 mg/kg, about 0.5 mg/kg-about 0.55 mg/kg, about 0.56 mg/kg-about 0.6 mg/kg, about 0.6 mg/kg-about 0.65 mg/kg, about 0.66 mg/kg-about 0.7 mg/kg, about 0.7 mg/kg-about 0.75 mg/kg, about 0.76 mg/kg-about 0.8 mg/kg, about 0.8 mg/kg-about 0.85 mg/kg, about 0.86 mg/kg-about 0.9 mg/kg, about 0.9 mg/kg-about 0.95 mg/kg, about 0.96 mg/kg-about 1 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, or about 35 mg/kg. In some embodiments, the subject is human and the first and/or second effective dose of dexamethasone is from about 0.16 mg/kg to about 8 mg/kg.

In any and all embodiments of the methods disclosed herein, the subject is diagnosed with, or is suspected of having cancer. The cancer or tumor may be a carcinoma, sarcoma, a melanoma, or a hematopoietic cancer. Examples of cancer include, but are not limited to, osteosarcoma, Ewing's sarcoma, adrenal cancers, bladder cancers, blood cancers, bone cancers, brain cancers, breast cancers including triple negative breast cancer, carcinoma, cervical cancers, colon cancers, colorectal cancers, corpus uterine cancers, ear, nose and throat (ENT) cancers, endometrial cancers, esophageal cancers, gastrointestinal cancers including gastric cancer, head and neck cancers, Hodgkin's disease, intestinal cancers, kidney cancers, larynx cancers, leukemias (including acute and chronic leukemias), liver cancers, lymph node cancers, lymphomas, lung cancers (including non-small cell lung cancer), melanomas, mesothelioma, myelomas (including multiple myeloma), nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof. In certain embodiments, the cancer is a relapsed or refractory cancer. In some embodiments, the cancer is resistant to radiation therapy, chemotherapy or immunotherapy. In certain embodiments, the subject is human. Additionally or alternatively, in some embodiments, the subject is non-responsive to at least one prior line of cancer therapy such as radiation therapy, chemotherapy, or immunotherapy.

Additionally or alternatively, in some embodiments, the subject exhibits decreased tumor growth, reduced tumor proliferation, lower tumor burden, or increased survival after administration of dexamethasone and an anti-CD3 multi-specific antibody of the present technology or an EAT of the present technology. Additionally or alternatively, in some embodiments of the combination therapy methods disclosed herein, the time to response and/or duration of response is improved relative to that observed with dexamethasone monotherapy, or monotherapy with an anti-CD3 multi-specific antibody or EAT of the present technology.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the EATs or anti-CD3 multi-specific antibodies described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In one aspect, the present disclosure provides a method for increasing the efficacy of T-cell based immunotherapy in a subject suffering from cancer comprising: administering to the subject an effective amount of dexamethasone and an effective amount of any and all embodiments of the anti-CD3 multi-specific antibody disclosed herein or any and all embodiments of the EATs disclosed herein. Examples of T-cell based immunotherapy include T cell engaging multi-specific antibodies, native T cells, non-autologous T cells, and CAR T cells.

Additionally or alternatively, in some embodiments, the methods of the present technology further comprise separately, simultaneously, or sequentially administering an additional cancer therapy. Examples of additional cancer therapies include, but are not limited to, chemotherapy, radiation therapy, immunotherapy, tumor-specific monoclonal antibodies, anti-cancer nucleic acids or proteins, anti-cancer viruses or microorganisms, and any combinations thereof. In some embodiments, the additional cancer therapy is an immune checkpoint inhibitor selected from among pembrolizumab, nivolumab, cemiplimab, atezolizumab, avelumab, durvalumab, and ipilimumab. In other embodiments, the additional cancer therapy comprises one or more of an anti-Ly6G antibody, an anti-GR-1 antibody, an anti-Ly6C antibody, an anti-CSF-1R antibody, Clodronate, a VEGF inhibitor and a VEGFR inhibitor.

Exemplary VEGF inhibitors include, but are not limited to, Linifanib (ABT-869, Abbott), AEE-788 (Novartis) (also called AE-788 and NVP-AEE-788), axitinib (AG-13736, (Pfizer) (also called AG-013736), AG-028262 (Pfizer), Angiostatin (EntreMed) (also called CAS Registry Number 86090-08-6, K1-4, and rhuAngiostatin, among others), Avastin™ (Genentech) (also called bevacizumab, R-435, rhuMAB-VEGF, and CAS Registry Number 216974-75-3), ranibizumab (Lucentis, Genentech), Vanucizumab, Brolucizumab, hPV19, IBI305, AVE-8062 (Ajinomoto Co. and Sanofi-aventis) (also called AC-7700 and combretastatin A4 analog, among others), Cediranib (AZD-2171, AstraZeneca), Nexavar® (Bayer AG and Onyx) (also called CAS Registry Number 284461-73-0, BAY-43-9006, raf kinase inhibitor, sorafenib, sorafenib analogs, and IDDBCP150446), BMS-387032 (Sunesis and Bristol-Myers Squibb) (also called SNS-032 and CAS Registry Number 345627-80-7), CEP-7055 (Cephalon and Sanofi-aventis) (also called CEP-11981 and SSR-106462), Dovitinib (CHIR-258, Chiron) (also called CAS Registry Number 405169-16-6, GFKI, and GFKI-258), CP-547632 (OSI Pharmaceuticals and Pfizer) (also called CAS Registry Number 252003-65-9), CP-564959, Lenvatinib (E-7080, Eisai Co.) (also called CAS Registry Number 417716-92-8 and ER-203492-00), pazopanib (GW786034, GlaxoSmithKline), GW-654652 (GlaxoSmithKline) and closely related indazolylpyrimidine Kdr inhibitors, IMC-1C11 (ImClone) (also called DC-101 and c-p1C11), Tivozanib (KRN-951, Kirin Brewery Co.) and other closely related quinoline-urea VEGF inhibitors, PKC-412 (Novartis) (also called CAS Registry Number 120685-11-2, benzoylstaurosporine, CGP-41251, midostaurin, and STI-412), PTK-787 (Novartis and Schering) (also called CAS Registry Numbers 212141-54-3 and 212142-18-2, PTK/ZK, PTK-787/ZK-222584, ZK-22584, VEGF-TKI, VEGF-RKI, PTK-787A, DE-00268, CGP-79787, CGP-79787D, vatalanib, ZK-222584) and closely related anilinophthalazine derivative VEGF inhibitors, SU11248 (Sugen and Pfizer) (also called SU-11248, SU-011248, SU-11248J, Sutent®, and sunitinib malate), SU-5416 (Sugen and Pfizer/Pharmacia) (also called CAS Registry Number 194413-58-6, semaxanib, 204005-46-9), Orantinib (SU-6668, Sugen and Taiho) (also called CAS Registry Number 252916-29-3, SU-006668, and TSU-68, among others) and closely related VEGF inhibitors as described in WO-09948868, WO-09961422, and WO-00038519 (which are hereby incorporated by reference in their entireties), VEGF Trap (Regeneron and Sanofi-aventis) (also called AVE-0005 and Systemic VEGF Trap, among others) and closely related VEGF inhibitors as described in WO-2004110490, which is hereby incorporated by reference in its entirety, Thalidomide (Celgene) (also called CAS Registry Number 50-35-1, Synovir, Thalidomide Pharmion, and Thalomid), Tesevatinib (XL-647, Exelixis) (also called EXEL-7647), XL-999 (Exelixis) (also called EXEL-0999), Foretinib (XL-880, Exelixis) (also called EXEL-2880), Vandetanib (ZD-6474, AstraZeneca) (also called CAS Registry Number 443913-73-3, Zactima, and AZD-6474) and closely related anilinoquinazoline VEGF inhibitors, and ZK-304709 (Schering) (also called ZK-CDK, MTGI) and other closely related compounds including the indirubin derivative VEGF inhibitors described in WO-00234717, WO-02074742, WO-02100401, WO-00244148, WO-02096888, WO-03029223, WO-02092079, and WO-02094814 which are hereby incorporated by reference in their entireties. Other VEGF inhibitors useful in the methods of the present technology include: (a) a compound described in US2003/0125339 or U.S. Pat. No. 6,995,162 which is herein incorporated by reference in its entirety, particularly in parts disclosing VEGF inhibitors (e.g., 4TBPPAPC); (b) a substituted alkylamine derivative described in US2003/0125339 or US2003/0225106 or U.S. Pat. No. 6,995,162 or U.S. Pat. No. 6,878,714 each of which is herein incorporated by reference in its entirety, particularly in parts disclosing VEGF inhibitors (e.g., AMG 706); and (c) VEGF inhibitors as described in US2006/0241115, including those of Formula IV therein.

In any and all embodiments of the methods disclosed herein, the dexamethasone and the anti-CD3 multi-specific antibody or EAT are administered separately, sequentially, or simultaneously. The dexamethasone and the anti-CD3 multi-specific antibody or EAT may be administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, topically, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Formulations including dexamethasone, an anti-CD3 multi-specific antibody, or EAT disclosed herein may be designed to be short-acting, fast-releasing, or long-acting. In other embodiments, compounds can be administered in a local rather than systemic means, such as administration (e.g., by injection) at a tumor site. In certain embodiments, the methods of the present technology further comprise administering a cytokine to the subject, optionally wherein the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2. The cytokine may be administered prior to, during, or subsequent to administration of the EAT.

Additionally or alternatively, in some embodiments of the methods disclosed herein, the dexamethasone can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) the administration of the anti-CD3 multi-specific antibody or EAT to a subject suffering from cancer.

In some embodiments, the dexamethasone and anti-CD3 multi-specific antibody or EAT are administered to a subject, for example, a mammal, such as a human, in a sequence and within a time interval such that the therapeutic agent that is administered first acts together with the therapeutic agent that is administered second to provide greater benefit than if each therapeutic agent were administered alone. For example, the dexamethasone and the anti-CD3 multi-specific antibody or EAT can be administered at the same time or sequentially in any order at different points in time; however, if not administered at the same time, the dexamethasone and anti-CD3 multi-specific antibody or EAT are administered sufficiently close in time so as to provide the desired therapeutic or prophylactic effect of the combination of the multiple therapeutic agents. In one embodiment, the dexamethasone and the anti-CD3 multi-specific antibody or EAT exert their effects at times which overlap. In some embodiments, the dexamethasone and the anti-CD3 multi-specific antibody or EAT each are administered as separate dosage forms, in any appropriate form and by any suitable route. In other embodiments, the dexamethasone and anti-CD3 multi-specific antibody or EAT are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, dexamethasone or anti-CD3 multi-specific antibody or EAT may be administered daily, weekly, biweekly, or monthly for a particular period of time. A dexamethasone or anti-CD3 multi-specific antibody or EAT may be dosed daily over a 14 day time period, or twice daily over a seven day time period. The dexamethasone or anti-CD3 multi-specific antibody or EAT may be administered daily for 7 days.

Alternatively, dexamethasone or anti-CD3 multi-specific antibody or EAT may be administered daily, weekly, biweekly, or monthly for a particular period of time followed by a particular period of non-treatment. In some embodiments, the dexamethasone or anti-CD3 multi-specific antibody or EAT may be administered daily for 14 days followed by seven days of non-treatment, and repeated for two more cycles of daily administration for 14 days followed by seven days of non-treatment. In some embodiments, the dexamethasone or anti-CD3 multi-specific antibody or EAT can be administered twice daily for seven days followed by 14 days of non-treatment, which may be repeated for one or two more cycles of twice daily administration for seven days followed by 14 days of non-treatment.

In some embodiments, the dexamethasone or anti-CD3 multi-specific antibody or EAT is administered daily over a period of 14 days. In another embodiment, the dexamethasone or anti-CD3 multi-specific antibody or EAT is administered daily over a period of 12 days, or 11 days, or 10 days, or nine days, or eight days. In another embodiment, the dexamethasone or anti-CD3 multi-specific antibody or EAT is administered daily over a period of seven days. In another embodiment, the dexamethasone or anti-CD3 multi-specific antibody or EAT is administered daily over a period of six days, or five days, or four days, or three days.

In some embodiments, individual doses of the dexamethasone and anti-CD3 multi-specific antibody or EAT are administered within a time interval such that the multiple therapeutic agents can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the subject. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the subject has achieved a complete response or a partial response, at which point the treatment may be stopped. Alternatively, the period of treatment in cycling therapy may continue until the subject has achieved a complete response or a partial response, at which point the period of treatment may continue for a particular number of cycles. In some embodiments, the length of the period of treatment may be a particular number of cycles, regardless of subject response. In some other embodiments, the length of the period of treatment may continue until the subject relapses.

In some embodiments, dexamethasone and the anti-CD3 multi-specific antibody or EAT are cyclically administered to a subject. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, dexamethasone is administered for a particular length of time prior to administration of the T cell engaging multi-specific antibody. For example, in a 21-day cycle, dexamethasone may be administered on days 1 to 5, days 1 to 7, days 1 to 10, or days 1 to 14, and the anti-CD3 multi-specific antibody or EAT may be administered on days 6 to 21, days 8 to 21, days 11 to 21, or days 15 to 21.

In some embodiments, the dexamethasone and anti-CD3 multi-specific antibody or EAT each are administered at a dose and schedule typically used for that agent during monotherapy. In other embodiments, when the dexamethasone is administered in combination with an anti-CD3 multi-specific antibody or EAT disclosed herein, one or more of the agents can advantageously be administered at a lower dose than typically administered when the agent is used during monotherapy, such that the dose falls below the threshold that an adverse side effect is elicited.

The therapeutically effective amounts or suitable dosages of dexamethasone and the anti-CD3 multi-specific antibody or EAT in combination depends upon a number of factors, including the nature of the severity of the condition to be treated, the particular inhibitor, the route of administration and the age, weight, general health, and response of the individual subject. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of dexamethasone can generally range, in single or divided or multiple doses, from about 10% to about 120% of the maximum tolerated dose as a single agent. In certain embodiments, the suitable dosages of dexamethasone are from about 20% to about 100% of the maximum tolerated dose as a single agent. In other embodiments, the suitable dosages of dexamethasone are from about 25% to about 90% of the maximum tolerated dose as a single agent. In some embodiments, the suitable dosages of dexamethasone are from about 30% to about 80% of the maximum tolerated dose as a single agent. In other embodiments, the suitable dosages of dexamethasone are from about 40% to about 75% of the maximum tolerated dose as a single agent. In some embodiments, the suitable dosages of dexamethasone are from about 45% to about 60% of the maximum tolerated dose as a single agent. In other embodiments, suitable dosages of dexamethasone are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of the anti-CD3 multi-specific antibody or EAT can generally range, in single or divided or multiple doses, from about 10% to about 120% of the maximum tolerated dose as a single agent. In certain embodiments, the suitable dosages of the anti-CD3 multi-specific antibody or EAT are from about 20% to about 100% of the maximum tolerated dose as a single agent. In some other embodiments, the suitable dosages of the anti-CD3 multi-specific antibody or EAT are from about 25% to about 90% of the maximum tolerated dose as a single agent. In some other embodiments, the suitable dosages of the anti-CD3 multi-specific antibody or EAT are from about 30% to about 80% of the maximum tolerated dose as a single agent. In some other embodiments, the suitable dosages of the anti-CD3 multi-specific antibody or EAT are from about 40% to about 75% of the maximum tolerated dose as a single agent. In some other embodiments, the suitable dosages of the anti-CD3 multi-specific antibody or EAT are from about 45% to about 60% of the maximum tolerated dose as a single agent. In other embodiments, suitable dosages of the anti-CD3 multi-specific antibody or EAT are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

In one non-limiting example, the method of reducing tumor burden comprises administering an effective amount of dexamethasone, an effective amount of the presently disclosed EATs and an effective amount of the anti-CD3 multi-specific antibodies to the subject, thereby inducing tumor cell death in the subject. In some embodiments, the EATs and the anti-CD3 multi-specific antibody are administered at different times. For example, in some embodiments, the EATs are administered and then the anti-CD3 multi-specific antibody is administered. In some embodiments, the anti-CD3 multi-specific antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours, 72 hours, 96 hours, or longer after the administration of the EATs. In other embodiments, the anti-CD3 multi-specific antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours, 72 hours, 96 hours, or longer prior to the administration of the EATs.

Additionally or alternatively, in some embodiments, the methods of the present technology further comprise detecting the presence of a DOTA-based hapten in a subject that has been administered any embodiment of the anti-CD3 multi-specific antibody or ex vivo armed T cell described herein comprising (a) administering to the subject an effective amount of a DOTA-based hapten, wherein the DOTA-based hapten comprises a radionuclide, and is configured to localize to the anti-CD3 multi-specific antibody or ex vivo armed T cell; and (b) detecting the presence of the DOTA-based hapten in the subject by detecting radioactive levels emitted by the DOTA-based hapten that are higher than a reference value, wherein the anti-CD3 multi-specific antibody is configured to localize to a tissue expressing one or more target antigens recognized by the anti-CD3 multi-specific antibody or wherein the ex vivo armed T cell is configured to localize to a tissue expressing one or more target antigens recognized by any embodiment of the anti-CD3 multi-specific antibody disclosed herein that is present on the ex vivo armed T cell.

Additionally or alternatively, in some embodiments, the methods of the present technology further comprise detecting the presence of a DOTA-based hapten in a subject that has been administered a complex comprising any embodiment of the ex vivo armed T cell described herein or any embodiment of the anti-CD3 multi-specific antibody disclosed herein and a DOTA-based hapten including a radionuclide, comprising detecting the presence of the DOTA-based hapten in the subject by detecting radioactive levels emitted by the complex that are higher than a reference value, wherein the anti-CD3 multi-specific antibody is configured to localize to a tissue expressing one or more target antigens recognized by the anti-CD3 multi-specific antibody or the ex vivo armed T cell is configured to localize to a tissue expressing one or more target antigens recognized by any embodiment of the anti-CD3 multi-specific antibody disclosed herein that is present on the ex vivo armed T cell.

Additionally or alternatively, in some embodiments, the method further comprises quantifying radioactive levels emitted by the DOTA-based hapten or complex that is localized to the tumor and/or radioactive levels emitted by the DOTA-based hapten or the complex that is localized in one or more normal tissues or organs of the subject. In certain embodiments, the one or more normal tissues or organs are selected from the group consisting of heart, muscle, gallbladder, esophagus, stomach, small intestine, large intestine, liver, pancreas, lungs, bone, bone marrow, kidneys, urinary bladder, brain, skin, spleen, thyroid, and soft tissue. In any of the preceding embodiments, the method further comprises determining biodistribution scores by computing a ratio of the radioactive levels emitted by the DOTA-based hapten or complex that is localized to the tumor relative to the radioactive levels emitted by the DOTA-based hapten or complex that is localized in the one or more normal tissues or organs of the subject. Additionally or alternatively, the method further comprises calculating estimated absorbed radiation doses for the tumor and the one or more normal tissues or organs of the subject based on the biodistribution scores. In some embodiments, the method further comprises computing a therapeutic index for the DOTA-based hapten or complex based on the estimated absorbed radiation doses for the tumor and the one or more normal tissues or organs of the subject.

In some embodiments of the preceding methods disclosed herein, the radioactive levels emitted by the complex or the detectably labeled DOTA-based hapten are detected using positron emission tomography or single photon emission computed tomography. Additionally or alternatively, in some embodiments of the methods disclosed herein, the radioactive levels emitted by the complex or the radiolabeled DOTA-based hapten are detected between 2 to 120 hours after the complex or the radiolabeled DOTA-based hapten is administered. In certain embodiments of the methods disclosed herein, the radioactive levels emitted by the complex or the radiolabeled DOTA-based hapten are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Additionally or alternatively, in some embodiments of the methods disclosed herein, the ex vivo armed T cell, the anti-CD3 multi-specific antibody, the complex or the detectably labeled DOTA-based hapten is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the ex vivo armed T cell, the anti-CD3 multi-specific antibody, the complex or the detectably labeled DOTA-based hapten is administered into the cerebral spinal fluid or blood of the subject.

Examples of DOTA-based haptens useful in the methods disclosed herein include, but are not limited to, benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-N02-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA. In any and all embodiments of the methods disclosed herein, the subject is human.

### Kits of the Present Technology

The presently disclosed subject matter provides kits for the treatment of cancer and/or reducing CRS in a subject in need thereof. In certain embodiments, the kit comprises any and all embodiments of the anti-CD3 multi-specific antibody disclosed herein in unit dosage form, dexamethasone, and instructions for using the same to treat cancer. In certain embodiments, the kits further comprise instructions for arming T cells with the anti-CD3 multi-specific antibody. Additionally or alternatively, in some embodiments, the kits may further comprise instructions for isolating T cells from an autologous or non-autologous donor, and agents for culturing, differentiating and/or expanding isolated T cells in vitro such as cell culture media, CD3/CD28 beads, zoledronate, cytokines such as IL-2, IL-15 (e.g., IL15Rα-IL15 complex), buffers, diluents, excipients, and the like. Additionally or alternatively, in some embodiments, the kits comprise any and all embodiments of the EATs described herein, dexamethasone, and instructions for using the same to treat cancer in a subject in need thereof. The kits may further comprise pharmaceutically acceptable excipients, diluents, or carriers that are compatible with one or more kit components described herein. The instructions will generally include information about the use of the composition for the treatment or prevention of a neoplasia (e.g., solid tumor).

In any of the preceding embodiments of the kits disclosed herein, the kit comprises a sterile container which contains a therapeutic agent disclosed herein (e.g., any and all embodiments of the anti-CD3 multi-specific antibody or EATs described herein, and/or any and all embodiments of the dexamethasone disclosed herein); such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. Additionally or alternatively, in some embodiments, the instructions include at least one of the following: description of the therapeutic agent (e.g., any and all embodiments of the anti-CD3 multi-specific antibody or EATs described herein, and/or dexamethasone); dosage schedule and administration for treatment or prevention of a neoplasia (e.g., solid tumor) or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The present technology is further illustrated by the following Examples, which should not be construed as limiting in any way.

### Example 1: Materials and Methods

### Construction and Expression of BsAbs

The construction of GD2-BsAb was as described by Xu H, et al., Cancer Immunol Res 3: 266-277 (2015), the entirety of which is incorporated by reference herein. For each BsAb, scFv of huOKT3 was fused to the C-terminus of the light chain of human IgG1 via a C-terminal (G₄S)₃ linker (SEQ ID NO: 154) as described by Orcutt K D, et al., Protein Eng Des Sel 23(4):221-8 (2010), the entirety of which is incorporated by reference herein. N297A and K322A on Fc were generated with site-directed mutagenesis via primer extension in polymerase chain reactions as described by Reikofski J. and Tao B Y., Biotechnol Adv 10: 535-47 (1992). The nucleotide sequence encoding each BsAb was synthesized by GenScript and was subcloned into a mammalian expression vector. BsAb was produced using Expi293™ expression system (Thermo Fisher Scientific, Waltham, MA) separately. Antibodies were purified with protein A affinity column chromatography. The purity of these antibodies was evaluated by size-exclusion high-performance liquid chromatography (SE-HPLC).

### Cell Lines

Representative melanoma cell line M14 (UCLA-SO-M14), osteosarcoma cell line 143B (ATCC-CRL-8303) and small cell lung cancer (SCLC) cell line NCI-N417 were used. All the cell lines used were authenticated by short tandem repeat profiling with PowerPlex 1.2 System (Promega, Madison, WI, USA), and periodically tested for mycoplasma infection using MycoAlert™ PLUS Mycoplasma Detection Kit (Lonza, Basel, Switzerland). The cells were cultured in RPMI1640 (Sigma-Adlrich, St. Louis, MO) supplemented with 10% heat-inactivated fetal bovine serum (FBS, Life Technologies, Carlsbad, CA) at 37° C. in a 5% CO₂ humidified incubator. The luciferase-labeled osteosarcoma cell line 143BLuc and melanoma cell line M14Luc were generated by retroviral infection with an SFG-GF Luc vector. BsAb treatment and depleting antibodies

GD2-BsAb was given intravenously with effector T cells or as GD2-BsAb armed T cells (GD2-EATs). Ex vivo BsAb armed T cells (EATs) were generated in the following order: T cells isolated from peripheral blood mononuclear cells (PBMCs) were activated with CD3/CD28 Dynabeads (Invitrogen, Carlsbad, CA) for 7 to 14 days in the presence of 30 IU/mL of IL-2. Activated T cells were harvested and armed with each BsAb for 20 minutes at room temperature. After incubation, the T cells were washed with PBS twice. Properties of EATs were tested with cell surface density of BsAb using idiotype antibody and in vitro cytotoxicity against target antigens. Activated T cells or EATs were injected intravenously with 1000 IU of IL-2 given subcutaneously. For depleting tumor infiltrating myeloid cells and modulating tumor microvasculature, anti-mouse Gr-1 (BioXCell, Lebanon, NH), anti-mouse Ly6G (BioXCell, Lebanon, NH) and anti-mouse Ly6C antibody (BioXCell, Lebanon, NH), clodronate liposome (Liposoma B.V.), anti-VEGF-A (bevacizumab) and anti-mouse VEGFR2 antibody (BioXCell, Lebanon, NH), and dexamethasone were given intraperitoneally (ip) one day or one hour before each BsAb or EAT injection.

### Measurement of VEGF Serum Levels

Concentration of VEGF serum levels were measured using a commercially available enzyme-linked immunosorbent assay (ELISA) kit (R&D Systems, Minneapolis, MN) according to the manufacturer's instruction. Briefly, serum samples and standards were incubated for 2 hours in a microplate pre-coated with human or murine anti-VEGF monoclonal antibodies. After washing three times, enzyme-linked anti-VEGF polyclonal antibodies were added. Unbound antibodies were removed by washing. The intensity of the reaction was then revealed with tetramethylbenzidine and optical density was measured at 450 nm using a Vmax microplate reader and Soft MAX Pro software (Molecular Devices, Menlo Park, CA, USA). All samples were run in triplicate, and a standard curve was established for each assay. The sensitivity of the assay was <9.0 pg/ml. Inter- and intra-assay variations were <10%.

### Anti-Tumor Effect in Human Tumor Xenografts

All animal procedures were performed in compliance with Memorial Sloan Kettering Cancer Center's institutional Animal Care and Use Committee (IACUC) guidelines. BALB/c Rag2^{-/-}IL-2Rγc^{-/-} (BRG) mice were used in this study. Target antigen (+) patient-derived xenograft (PDX) was established from fresh surgical specimens with MSKCC IRB approval. Tumor cells in Matrigel (Corning Corp, Corning, NY) were implanted subcutaneously on the right flank of each mouse. Tumor size was measured using handheld Peira TM900 imaging device (Piera, Brussels, BE). Tumor growth curves and overall survival was analyzed, and the overall survival was defined as the time from start of treatment to when tumor volume reached 2000 mm³. To define the well-being of mice, CBC analyses, changes in body weight, behavior and physical appearance were monitored. All animal experiments were repeated twice more with different donor's T cells to ensure that the results were reliable.

### T Cell Transduction

T cells isolated from peripheral blood were stimulated with Dynabeads™ Human T-Activator CD3/CD28 for 24 hours. T cells were transduced with retroviral constructs containing tdTomato and click beetle red luciferase in RetroNectin-coated 6-well plates in the presence of IL-2 (100 IU/ml) and protamine sulfate (4 g/mL). Transduced T cells were cultured for 8 days before being used in animal experiments.

### Bioluminescence Imaging

Mice were anesthetized and imaged after intravenous injection of 3 mg of D-luciferin (Gold Biotechnology, St. Louis, MO) on different days post T cell injection. Images were acquired using IVIS Spectrum CT In Vivo Imaging System (Caliper Life Sciences, Waltham, MA). Bioluminescence images were overlaid with photographs, and regions of interest (ROI) were drawn based on the location and contour of tumor using Living image 2.60 (Xenogen, Alameda, CA). The total counts of photons (photon/s) were obtained. Bioluminescence signals (total flux, photon/s) before T cell injection were used as baselines.

### Immunohistochemistry and Immunofluorescence Staining

Immunohistochemistry (IHC) and immunofluorescence (IF) staining were performed at the MSK Molecular Cytology Core Facility using Discovery XT processor (Ventana Medical Systems, Oro Valley, AZ) as described by Xu H, et al., Cancer Immunol Res 3: 266-277 (2015), the entirety of which is incorporated herein by reference. Tumor samples were fixed and embedded in paraffin. Anti-human CD45, anti-human CD3, and anti-mouse CD68 antibodies were used, which was followed by biotinylated secondary antibody. The detection was performed using a DAB detection kit (Ventana Medical Systems, Oro Valley, AZ) or Alexa Fluor™ 488 or 568 Tyramide Reagent (Invitrogen, Carlsbad, CA). IHC images were captured from tumor sections using a Nikon ECLIPSE Ni-U microscope and NIS-Elements 4.0 imaging software. IF images were captured using Leica Inverted Confocal SP8 and processed with Imaris (Bitplane, Zurich, Switzerland). Antigen positive cells were counted with Qupath 0.1.2.

### Flow Cytometry Analysis and Cytokine Assay

For blood and tumor samples from mice, the following antibodies were purchased from Biolegend: anti-human CD45-APC (HI30), anti-human CD3-Percp/Cy5.5 (SK7), anti-human CD8-FITC, anti-human CD4-PE/Cy7, anti-mouse CD45-APC, anti-mouse CD45-Brilliant Violet 711™ (30-F11), anti-mouse CD11b-Brilliant Violet 570™ (M1/70), anti-mouse Ly6G-FITC, anti-mouse Ly6C-PerCP/Cy5.5, and anti-mouse F4/80-PE/Cy7. Statistical analysis

In vivo anti-tumor effect was compared by area under curve (AUC) and survival curves, which were calculated. Differences between samples indicated in the figures were tested for statistical significance by two-tailed Student's t-test for two sets of data while one-way ANOVA with Tukey's post hoc test was used to among three or more sets of data. All statistical analyses were performed using GraphPad Prism V.8.0 for Windows (GraphPad Software, La Jolla, CA, www.graphpad.com). P value <0.05 was considered statistically significant. Asterisks indicate that the experimental P value is statistically significantly different from the associated controls at *P<0.05; **P<0.01; ***P<0.001, ****P<0.0001.

### Example 2: Target Antigen Specific BsAb and T Cell Treatment have Profound Effect on Tumor Infiltrating Myeloid Cell (TIM) Populations

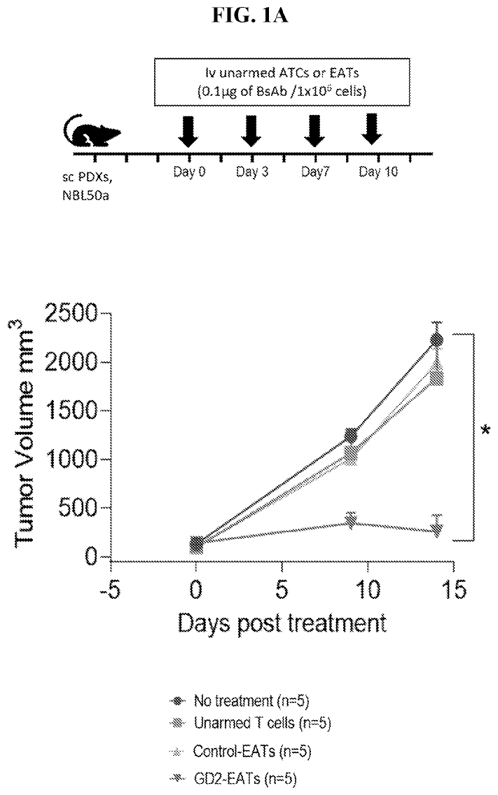

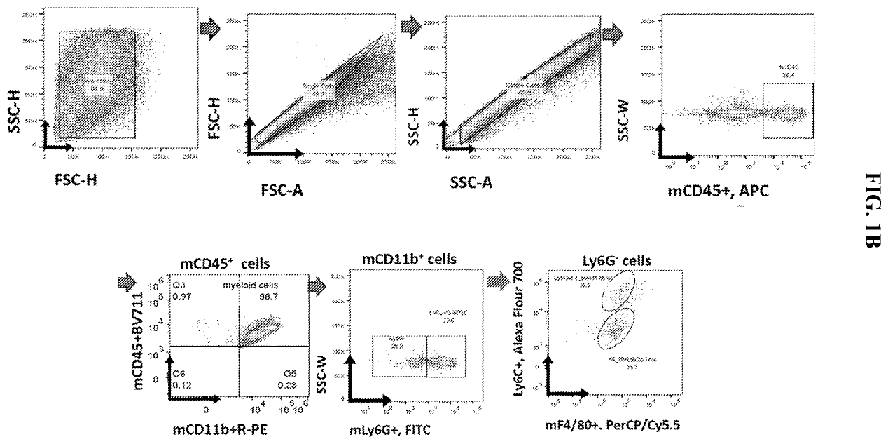

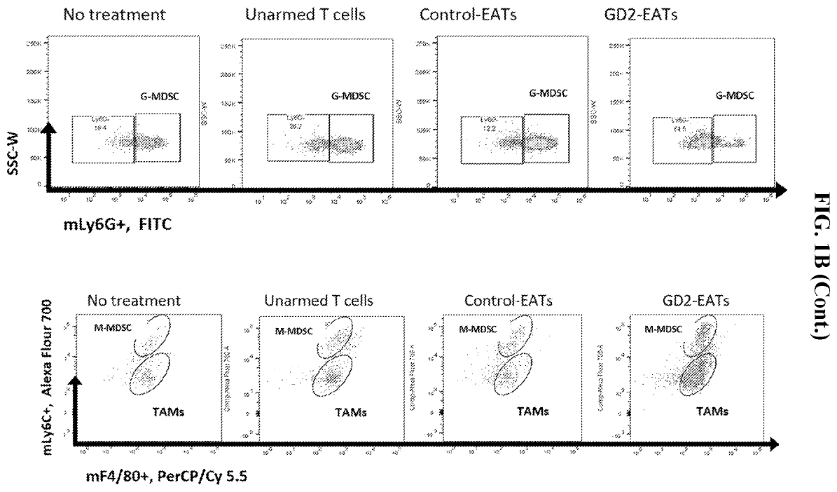

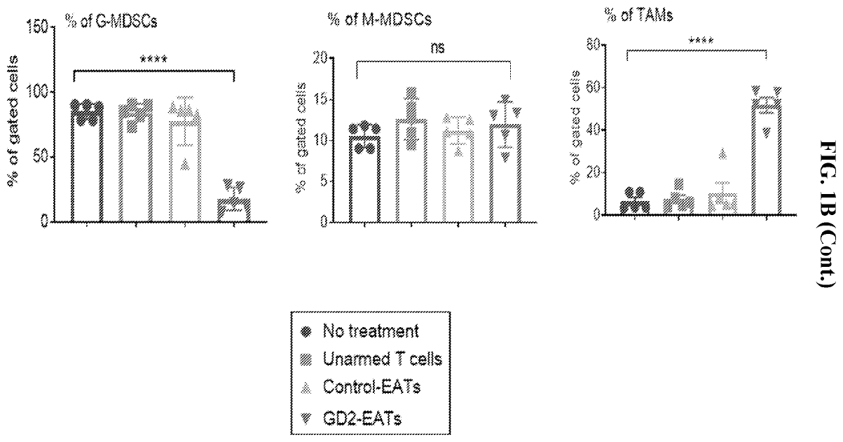

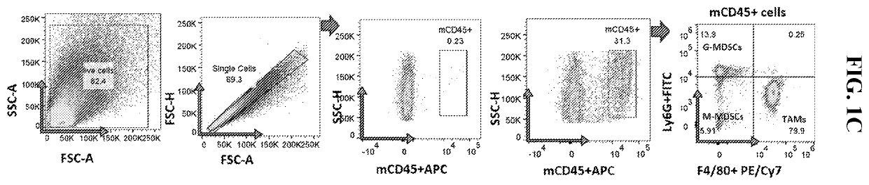

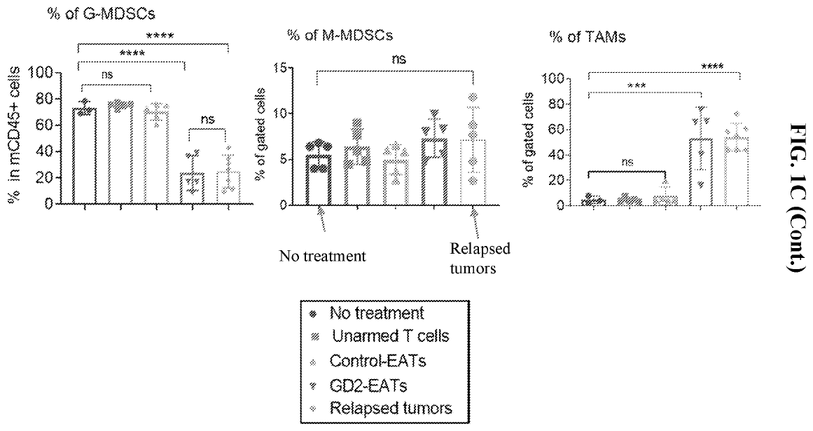

Ex vivo GD2-BsAb armed T cells (GD2-EATs) were generated and administered to treat GD2(+) neuroblastoma PDXs (FIG. 1A). In contrast to GD2-EATs, other control groups including no treatment, unarmed T cells, and control-BsAb armed EATs (control-EATs) failed to suppress tumor growth. When tumors were harvested on day 15 post treatment and analyzed by flow cytometry (FIG. 1B), those treated with GD2-EATs had substantially fewer mouse derived Ly6G⁺ MDSCs (mCD45⁺CD11b⁺Ly6G⁺F4/80⁻) and more Ly6G⁻F4/80⁺ TAMs (mCD45⁺CD11b⁺Ly6G⁻F4/80⁺) compared to control groups.

GD2-EATs suppressed neuroblastoma PDXs and prolonged mouse survival, but some tumors ultimately recurred. When those tumors were analyzed by flow cytometry (FIG. 1C), the majority had high frequencies of murine CD11b⁺Ly6G⁻F4/80⁺ TAMs and a few CD11b⁺Ly6G⁺F4/80⁻ G-MDSCs, in contrast to control groups which have a majority of G-MDSCs. This CD11b⁺Ly6G⁻F4/80⁺ TAM enrichment appeared during initial tumor response and again during tumor recurrence, but only in tumors responding to GD2-EATs.

### Example 3: Granulocytes MDSC (G-MDSC) Depletion Enhanced Anti-Tumor Effect of Target Antigen Specific BsAb and T Cell Treatment

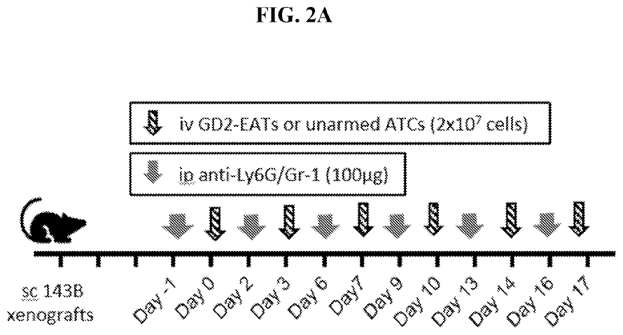

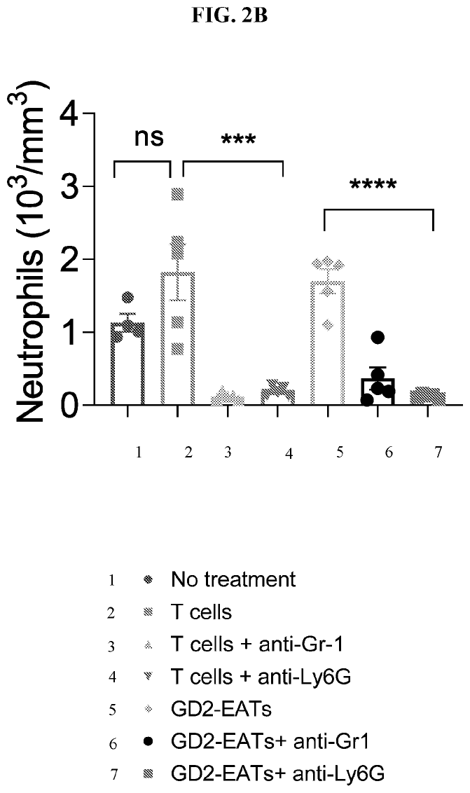

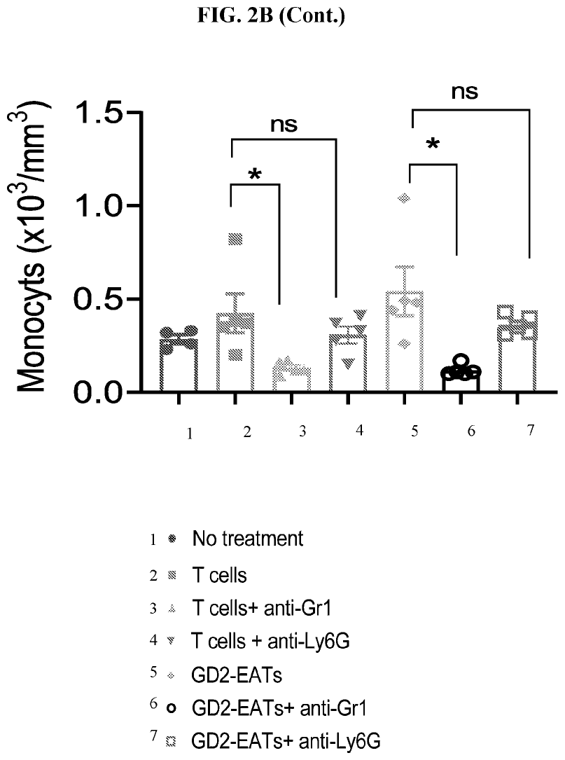

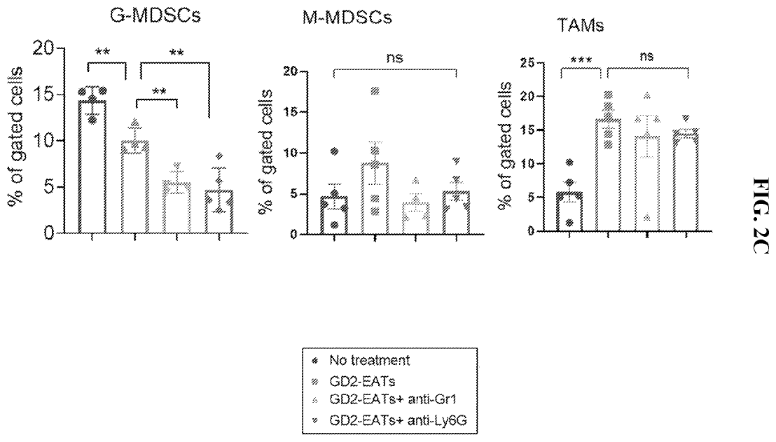

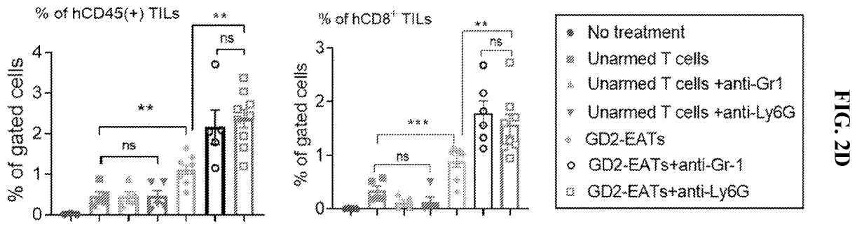

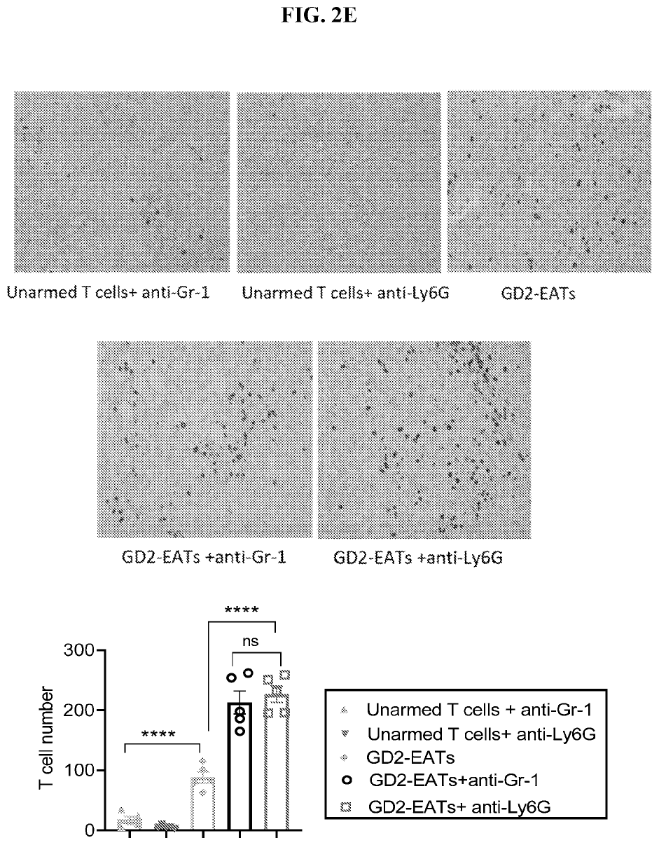

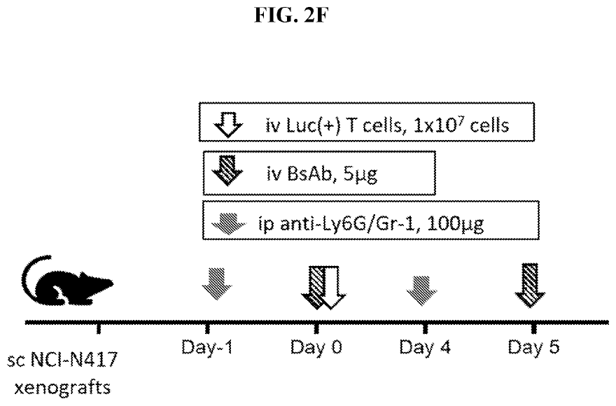

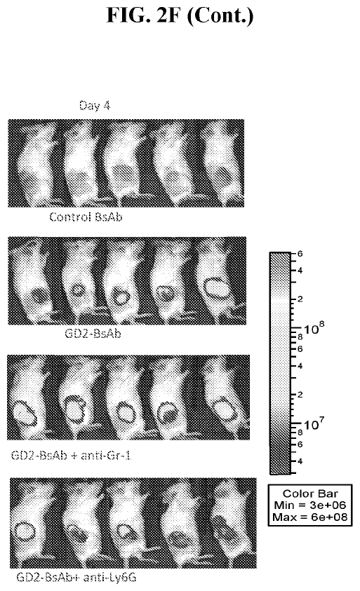

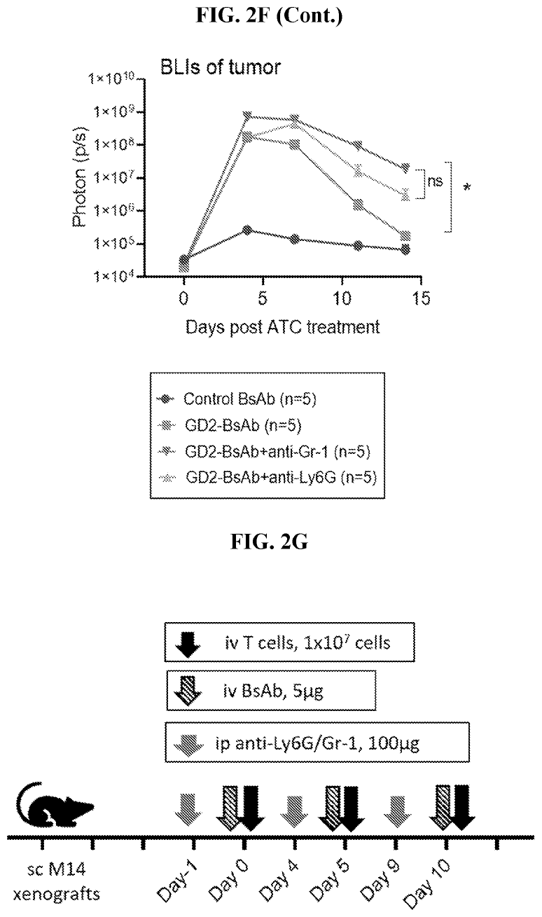

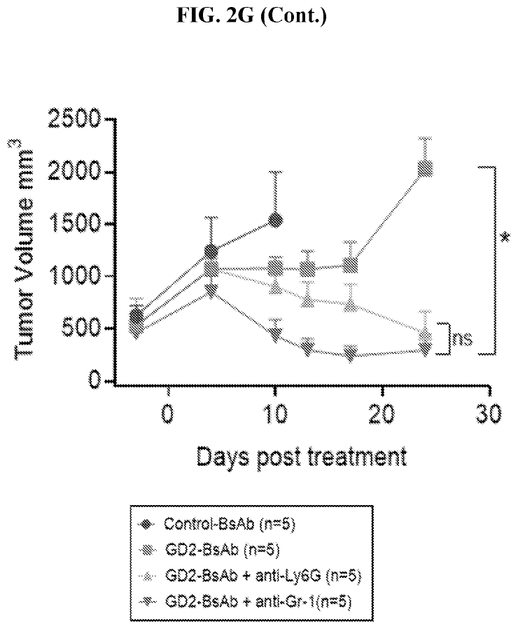

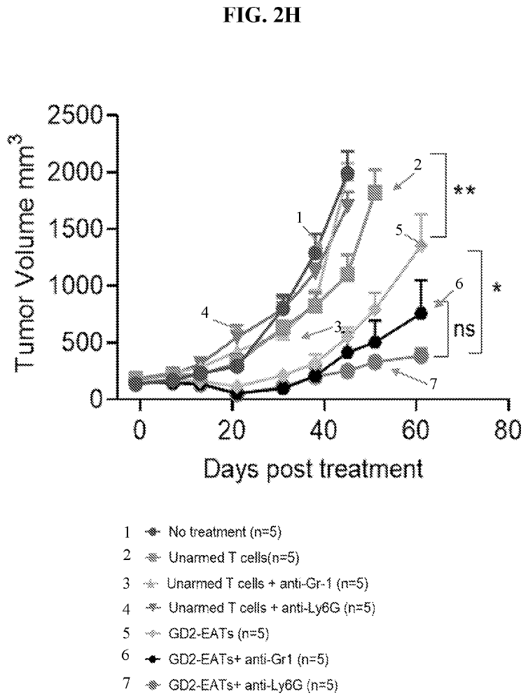

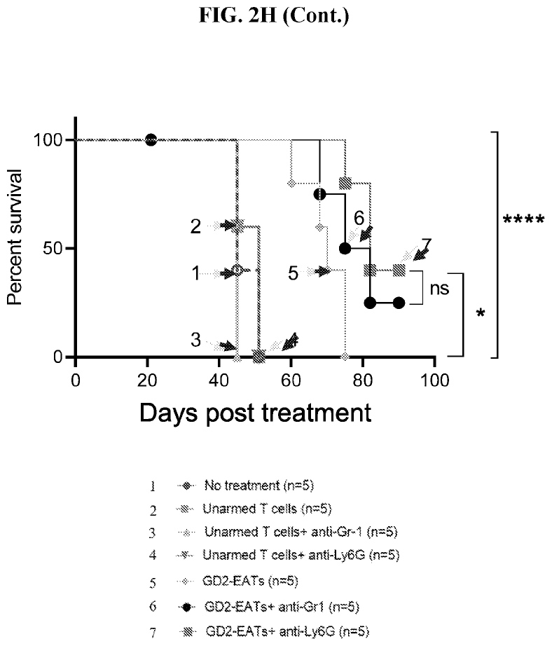

To test if removing MDSCs could enhance BsAb-directed T cell immune response, the GD2(+) osteosarcoma cell line 143B xenograft mouse model was used, where GD2-BsAb was substantially less effective than osteosarcoma PDXs. To deplete G-MDSCs, intraperitoneal (ip) injection of 100 g of anti-Ly6G or anti-GR-1 antibody was administered one day before each GD2-EAT treatment (FIG. 2A). After 2 doses of depleting antibodies, peripheral blood (PB) was collected for CBC analyses (FIG. 2B). Anti-Ly6G and anti-Gr-1 antibody successfully depleted neutrophils in circulation, and anti-GR-1 depleted monocytes as well.

Tumors were harvested on day 60 post-treatment and analyzed using flow cytometry. GD2-EATs reduced intratumoral murine G-MDSCs, and the addition of anti-Ly6G or anti-Gr-1 antibody further reduced the frequencies of G-MDSCs (mCD45⁺CD11b⁺Ly6G⁺Ly6C⁻ cells) in tumors compared to GD2-EATs alone (FIG. 2C). Inversely, human CD45(+) T cell infiltration in tumors was increased by G-MDSC depletion (FIG. 2D). GD2-EATs successfully infiltrated into tumors, and anti-Ly6G and anti-GR-1 antibody further enhanced T cell infiltration, especially for CD8(+) T cells. The difference in TIL frequencies among groups was confirmed by anti-human CD3 IHC staining (FIG. 2E).

To study the effect of G-MDSC depletion on BsAb-driven T cell trafficking, 1×10⁷ of luciferase labeled T cells [Luc(+) T cells] were administered iv into GD2(+) small cell lung cancer (SCLC) cell line (NCI-N417) xenografted mice, followed by GD2-BsAb (5μ/dose, iv twice weekly). Anti-Ly6G or anti-Gr-1 antibody was injected iv one day before each BsAb injection (FIG. 2F). Mean bioluminescence intensity (photon/sec) of TILs within tumors was compared among groups. In contrast to control BsAb, GD2-BsAb successfully drove Luc(+) T cells into tumors. The addition of anti-Gr-1 or anti-Ly6G antibody amplified the intensity of bioluminescence within tumors more than two-fold above those treated with GD2-BsAb alone. In addition, these G-MDSC depleting antibodies delayed bioluminescence signal loss. While the bioluminescence intensity by day 11 has dropped to 1% of its peak in the GD2-BsAb alone group, the addition of anti-Ly6G or anti-Gr-1 antibody enhanced Luc(+) T cell persistence: the intensities by day 11 was 10 to 50-fold higher compared to GD2-BsAb alone (P=0.0087).

The enhancement of T cell infiltration and persistence in tumors by granulocyte depletion translated to a substantial improvement in anti-tumor effect. In contrast to control-BsAb where tumors grew unabated, GD2-BsAb treatment suppressed tumor growth, and this anti-tumor response against melanoma cell line (M14) xenografts was much enhanced by G-MDSC depletion (P=0.0104) (FIG. 2G). The effect of G-MDSC depletion on anti-tumor response by GD2-EATs was confirmed in osteosarcoma cell line 143B xenograft model (FIG. 2A and FIG. 211). Anti-Gr-1 or anti-Ly6G antibody improved osteosarcoma tumor control compared to GD2-EATs alone (P=0.0384), leading to improved survival (P=0.0239).

### Example 4: Monocytes MDSC (M-MDSC) Depletion Enhanced Anti-Tumor Effect of Target Antigen Specific BsAb and T Cell Treatment

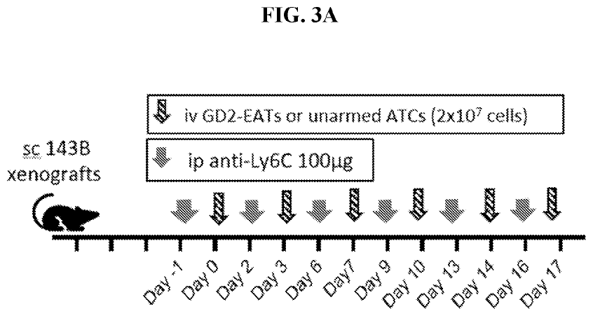

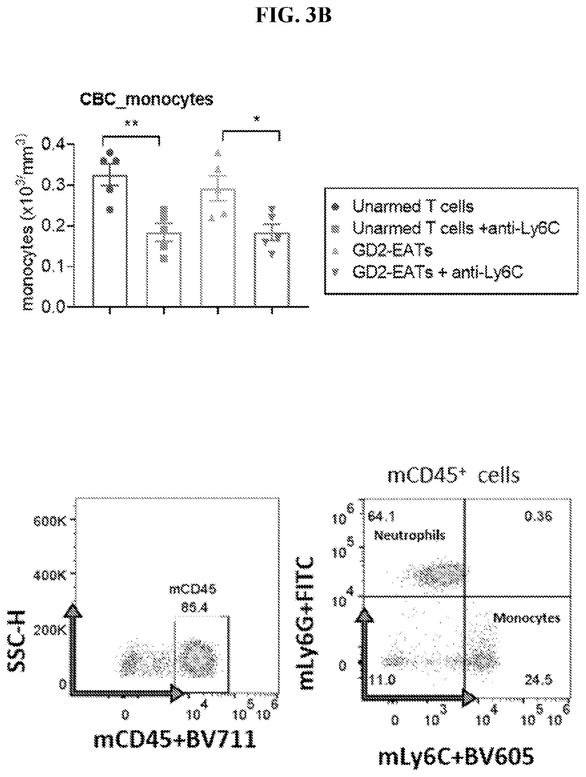

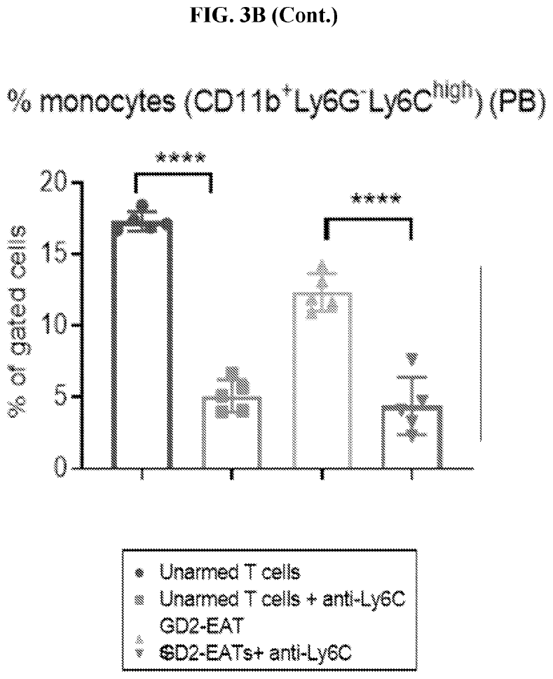

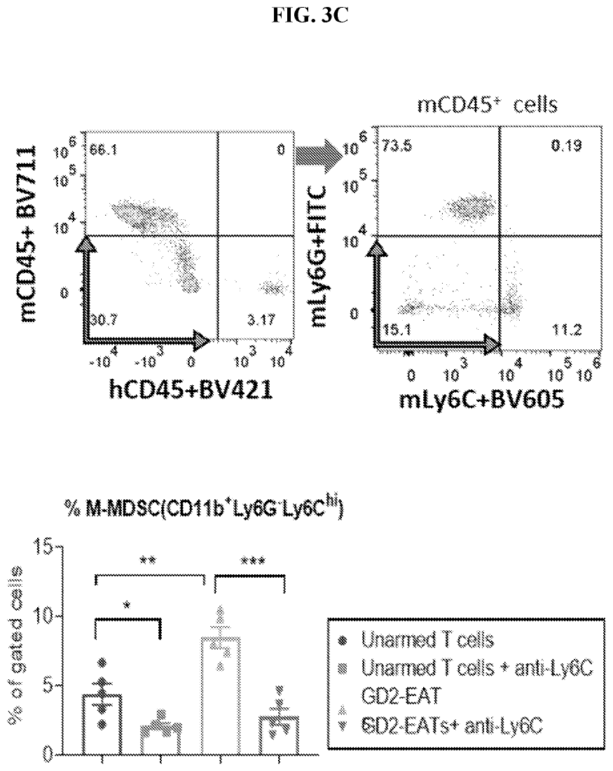

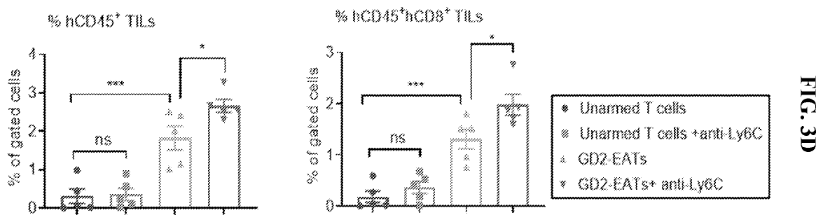

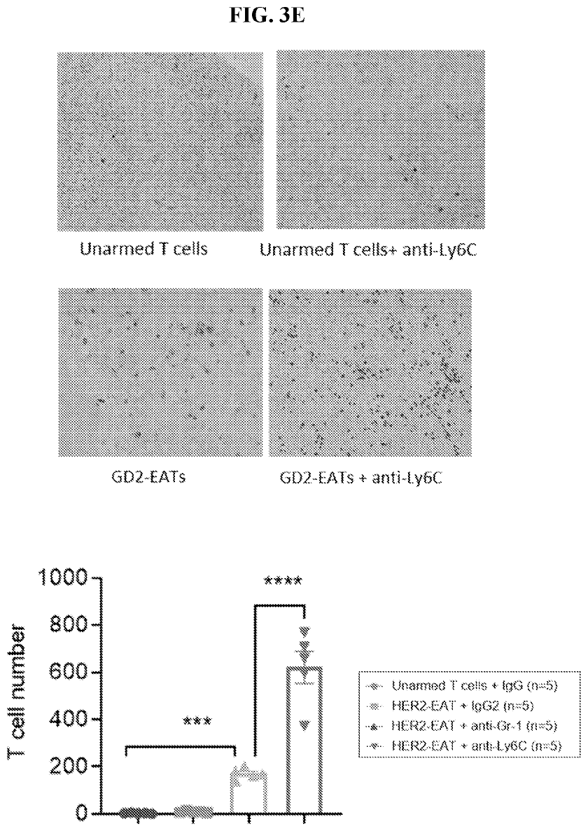

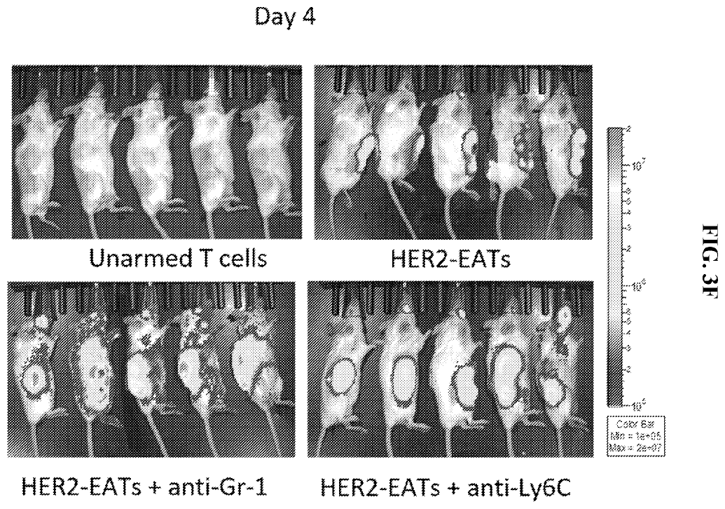

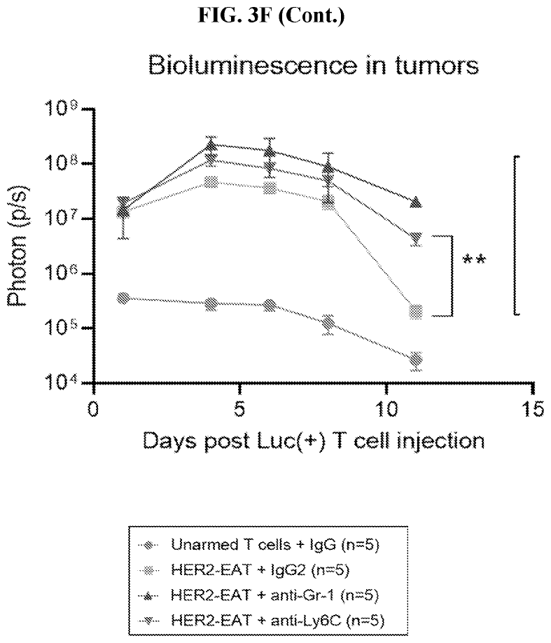

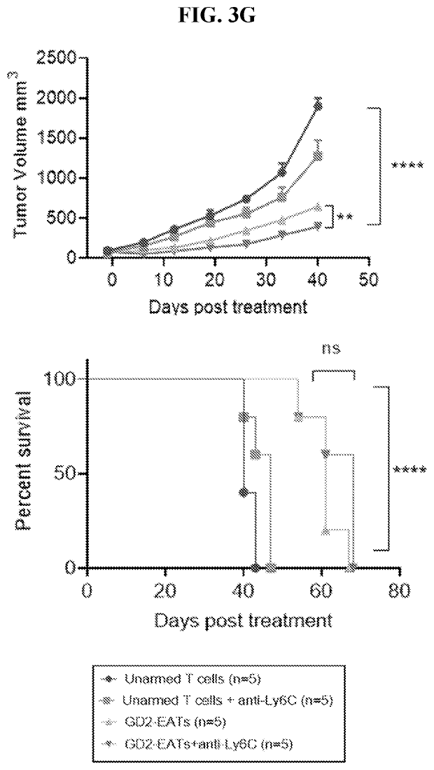

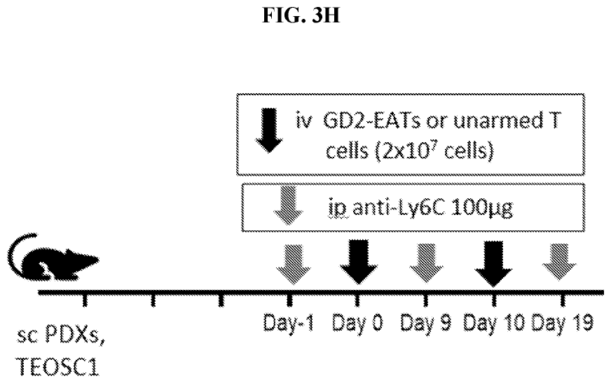

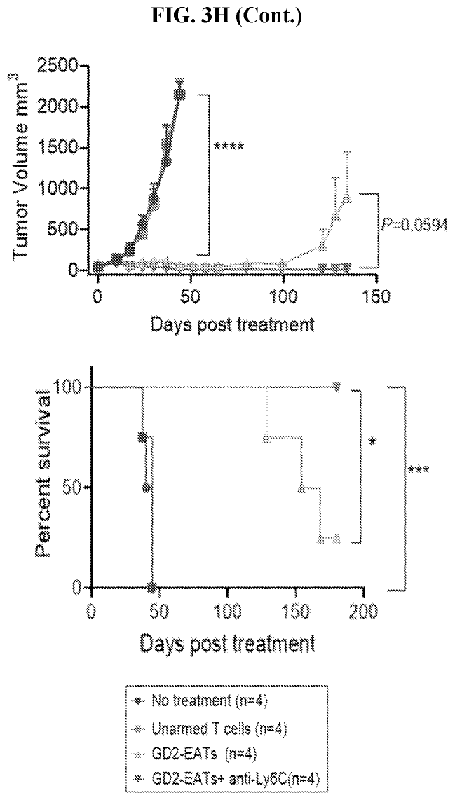

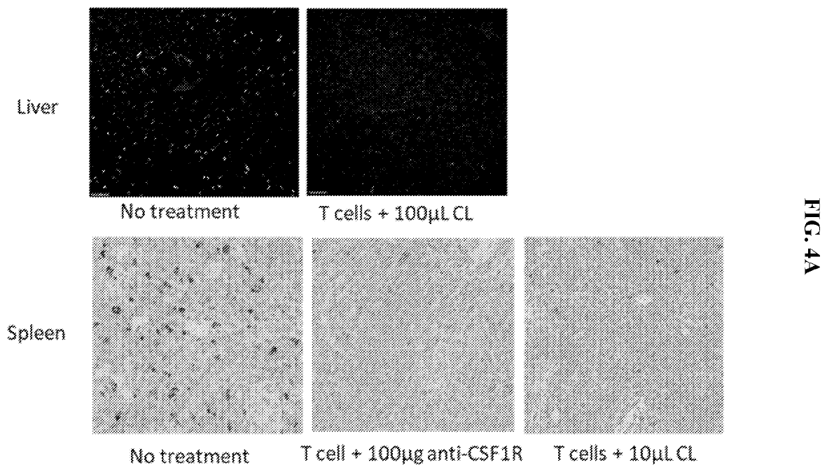

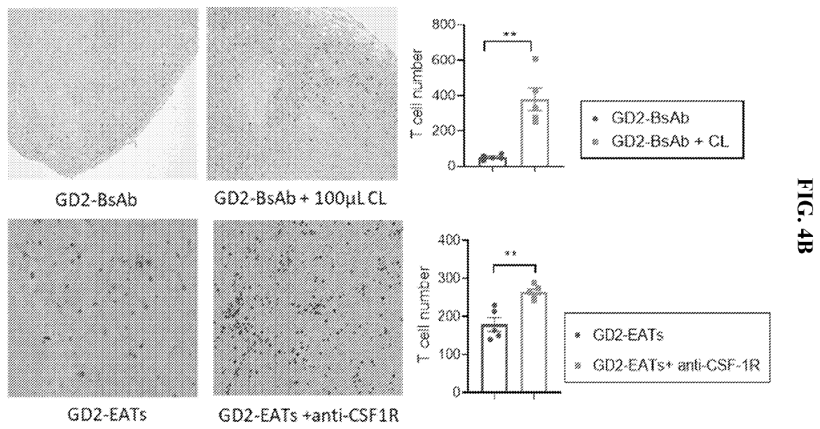

To test the role of M-MDSCs depletion, anti-Ly6C antibody was applied to the osteosarcoma 143B xenograft model, where mice were treated with iv GD2-EATs (3 doses/week, for 2 weeks). Anti-Ly6C antibody was injected 24 hours before each GD2-EATs (FIG. 3A). After 2 doses of anti-Ly6C antibody, CBC and flow cytometry analyses of PB confirmed the depletion of monocytes, i.e., CD11b⁺Ly6G⁻Ly6C^{hi} cell population, in the circulation (FIG. 3B). When tumors were analyzed on day 60 post treatment, anti-Ly6C antibody effectively depleted intratumoral M-MDSCs, i.e., mCD45⁺CD11b⁺Ly6G⁻Ly6C^{hi} cells, in contrast to those treated with unarmed T cells or GD2-EATs alone (FIG. 3C). In the presence of anti-Ly6C antibody, GD2-EATs treated tumors showed significantly more frequent TILs, i.e., hCD45(+) TILs and CD8(+) TILs, than tumors treated with GD2-EATs alone (FIG. 3D). IHC staining using anti-human CD3 antibody confirmed the increase of T cell infiltration in tumors by anti-Ly6C antibody (FIG. 3E).

To study the effect of M-MDSCs depletion on T cell trafficking into tumors, 100 g of anti-Ly6C antibody was administered iv one day before Luc(+) HER2-EATs injection into HER2(+) osteosarcoma PDX bearing mice (FIG. 3F). The Luc(+) HER2-EATs successfully trafficked into tumors, showing highest bioluminescence intensities around day 3 and 4, which dropped to 1% of the peak intensities by day 11. Anti-Ly6C delayed the dissipation of the Luc(+) HER2-EATs, whose bioluminescence intensities remained more than 10-fold higher compared to HER2-EATs alone on day 11 (P=0.0059).

This delayed clearance of T cells' bioluminescence signal in tumors was associated with improved tumor control. Osteosarcoma cell line 143B xenografts were treated with GD2-EATs and anti-Ly6C antibody (FIG. 3A). Anti-Ly6C combination with GD2-EATs improved tumor control compared to GD2-EATs alone (P=0.0061) (FIG. 3G). In a separate set of experiments using osteosarcoma PDX mouse model, while 3 of 4 mice treated with GD2-EATs experienced tumor relapse, none of 4 mice treated with GD2-EATs plus anti-Ly6C antibody relapsed (>180 days post treatment), demonstrating the role of depleting M-MDSCs on anti-tumor response by GD2-EATs (P=0.0401)(FIG. 311).

### Example 5: Macrophage MDSC (M-MDSC) Depletion Enhanced Anti-Tumor Effect of Target Antigen Specific BsAb and T Cell Treatment

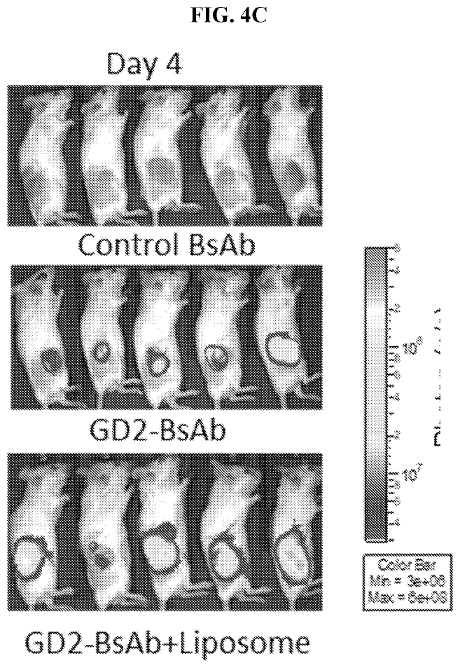

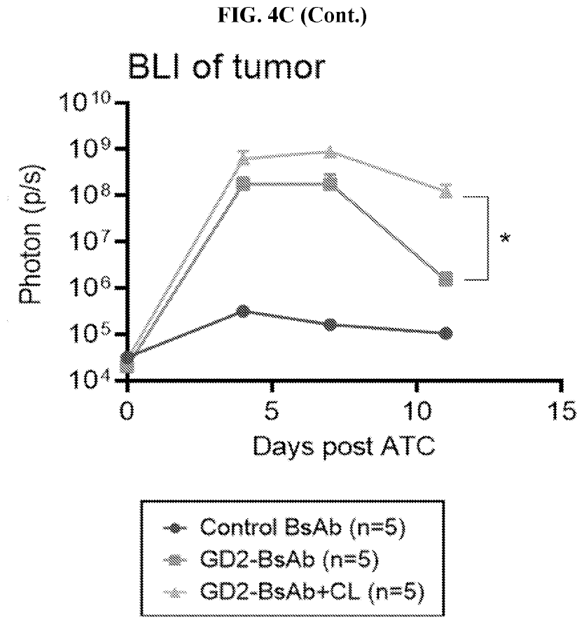

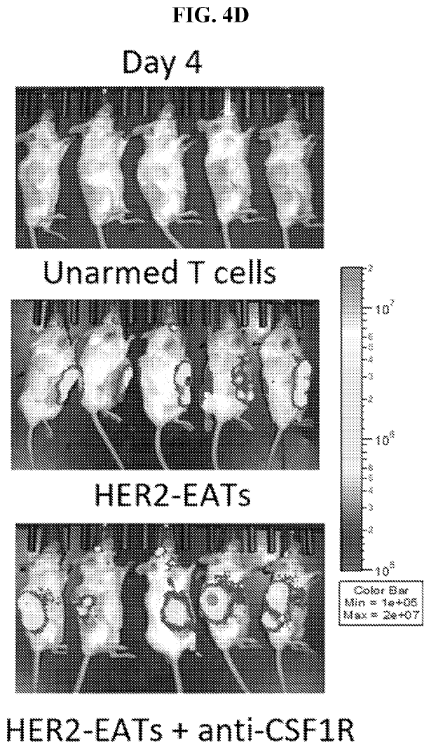

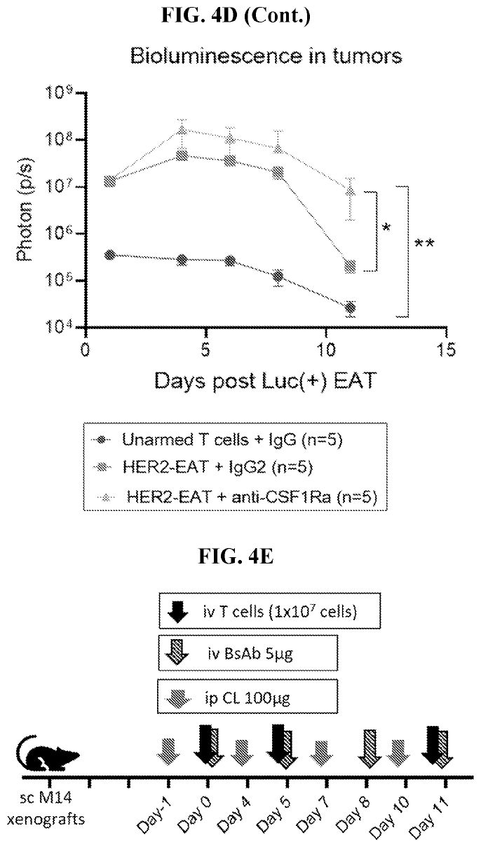

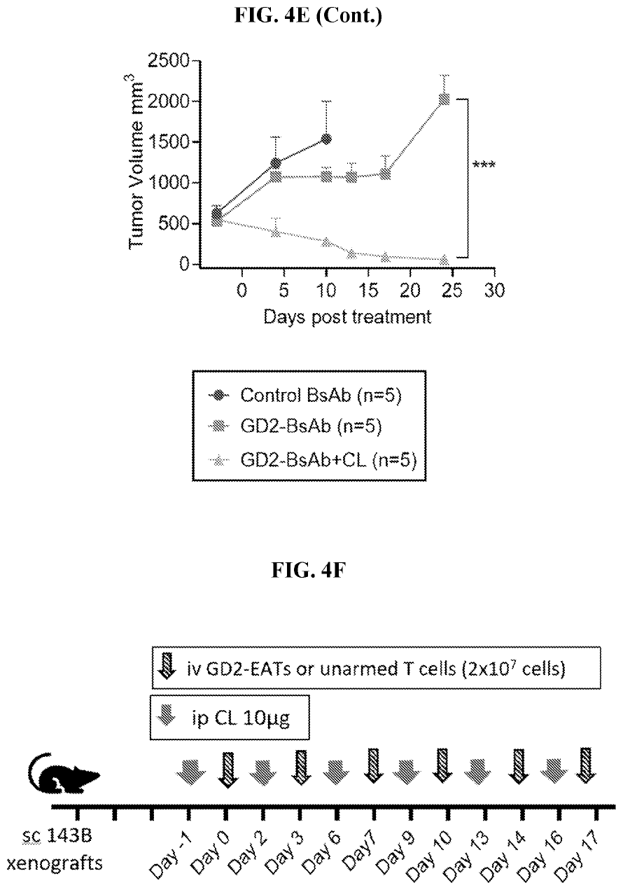

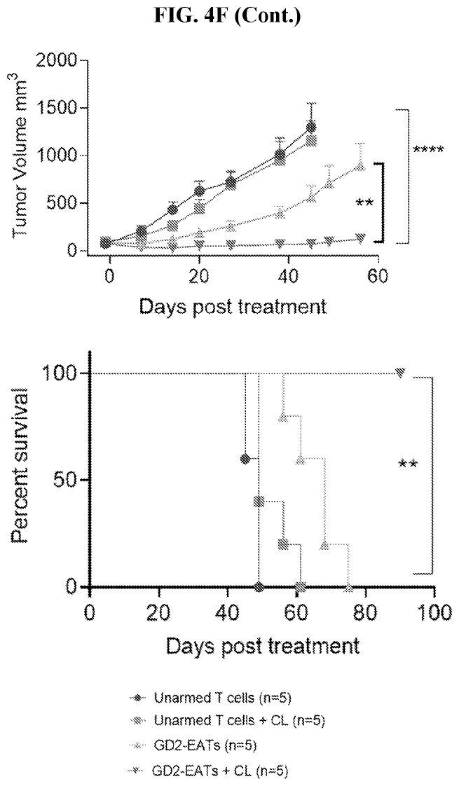

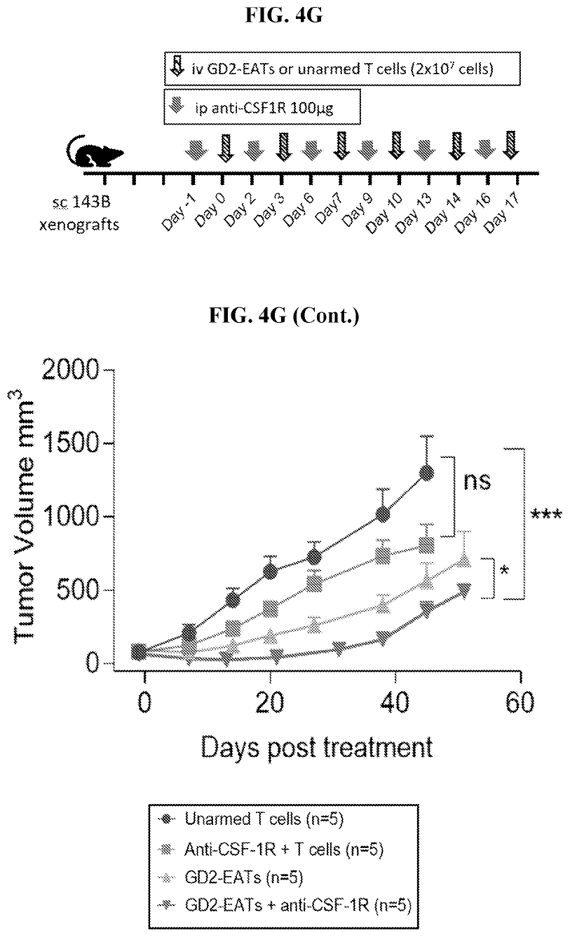

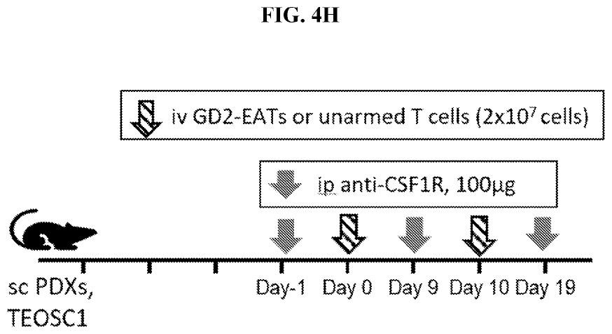

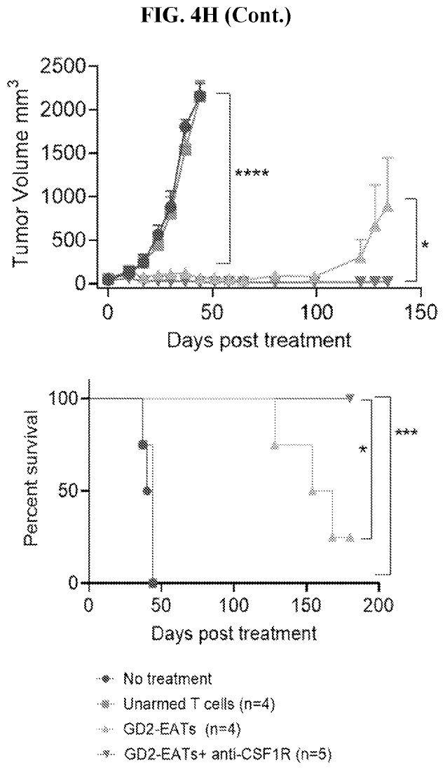

To study the role of tumor-associated macrophage (TAM) in tumor progression, mice were treated with either clodronate liposome (CL, 100 μL) or anti-CSF1R antibody (100 μg). After 2 doses of each treatment, livers and spleens were immunostained with anti-mouse CD68 antibody, and compared to control treatment (FIG. 4A). Either 100 μL of CL or 100 g of anti-CSF-1R antibody was able to deplete macrophages from the liver or spleen; a 10-fold lower dose (10 μL) of CL was also effective in depleting splenic macrophages.

Next, the effect of macrophage depletion on BsAb-driven T cell infiltration in tumors was studied (FIG. 4B). GD2-BsAb with or without CL was administered to GD2(+) M14 melanoma cell line xenografted mice. When tumors were stained with anti-human CD45 antibody, GD2-BsAb plus CL had significantly more TILs than GD2-BsAb alone (P=0.0010). T cell distribution within melanoma xenografts was more diffuse in the presence of CL, different from the restricted distribution towards the tumor margins when CL was not given. When anti-CSF1R antibody was administered to deplete macrophages in osteosarcoma 143B xenograft mouse model, it also enhanced GD2-BsAb-driven T cell infiltration in tumors. Four doses of anti-CSF1R antibody, each given 24 hours before GD2-EATs, increased CD3(+) TILs within tumors when compared to GD2-EATs alone (P=0.0024).

To evaluate the effect of macrophage depletion on T cell trafficking into tumors, 100 μL of CL was administered to mice bearing SCLC NCI-N417 xenografts with GD2-BsAb (5 g/dose) and Luc(+) T cells (1×10⁷ cells) (FIG. 4C). Contrast to control-BsAb, GD2-BsAb treated tumors showed high bioluminescence, and the signal intensities increased 10-fold in the presence of CL. While the bioluminescence in tumors treated with GD2-BsAb fell sharply after day 7, those in GD2-BsAb with CL was maintained through day 11, being 100-fold higher compared to GD2-BsAb alone (P=0.0209). The effect of anti-CSF1R on T cell trafficking was also evaluated using HER2(+) osteosarcoma PDX model (FIG. 4D). The bioluminescence for HER2-EATs plus anti-CSF1R persisted longer than those for HER2-EATs alone. In the presence of anti-CSF1R, the bioluminescence for HER2-EATs in tumors had increased 20-fold on day 15 (P=0.0222).

The enhanced TIL persistence by macrophage depletion also translated to improved tumor control. M14 xenografts treated with GD2-BsAb plus CL showed complete regression despite large tumor volume at treatment start, contrasting with GD2-BsAb alone where tumor growth was poorly controlled (FIG. 4E). This enhancing anti-tumor effect of CL was confirmed in osteosarcoma cell line xenograft model (FIG. 4F). Due to thromboembolic complications of 100 μL of CL, reduced dose (10 μL) of CL was administered without significant complication. The CL significantly improved anti-tumor response of GD2-EATs (P=0.0011) and prolonged survival (P=0.0021). The effect of anti-CSF1R was also confirmed in two different osteosarcoma xenograft models, 143B cell line xenografts (FIG. 4G) and TEOSC1 PDXs (FIG. 411). In both tumor models, macrophage depletion using anti-CSF1R improved tumor control, especially effective against TEOSC1 PDXs both in tumor response as well as in survival (P=0.0221).

### Example 6: Targeting VEGF/VEGFR to Modulate Anti-Tumor Immunity

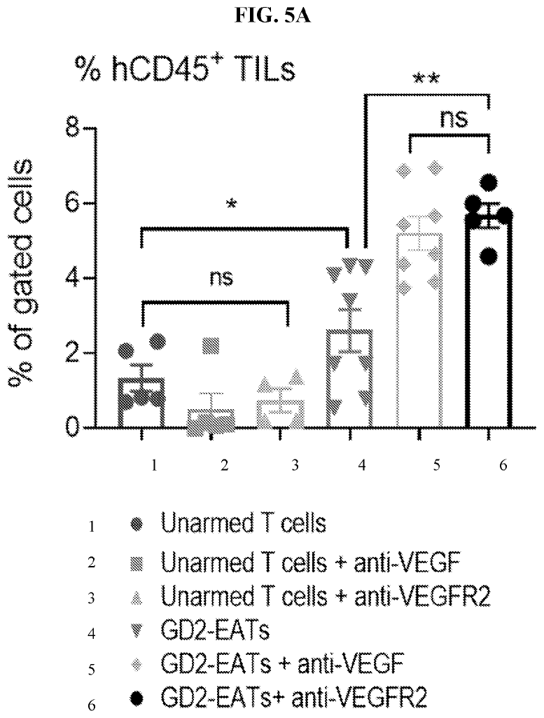

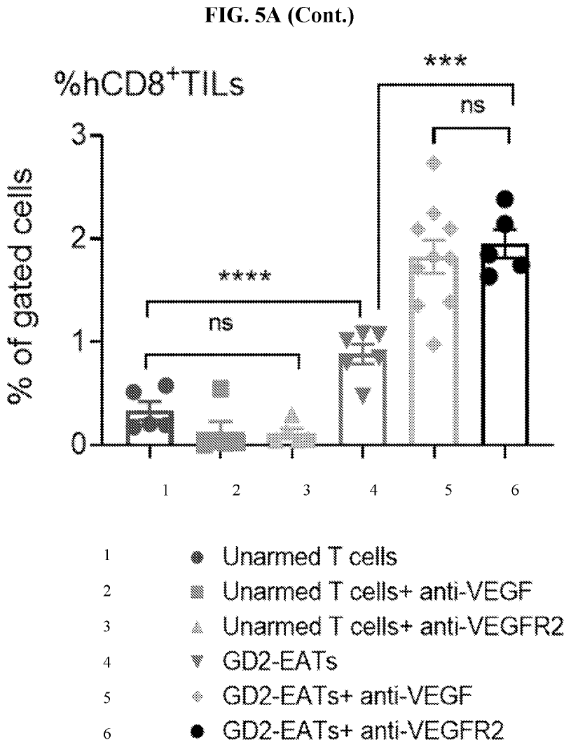

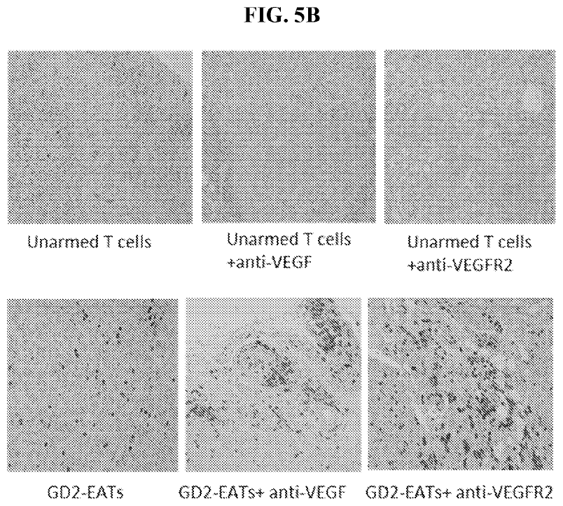

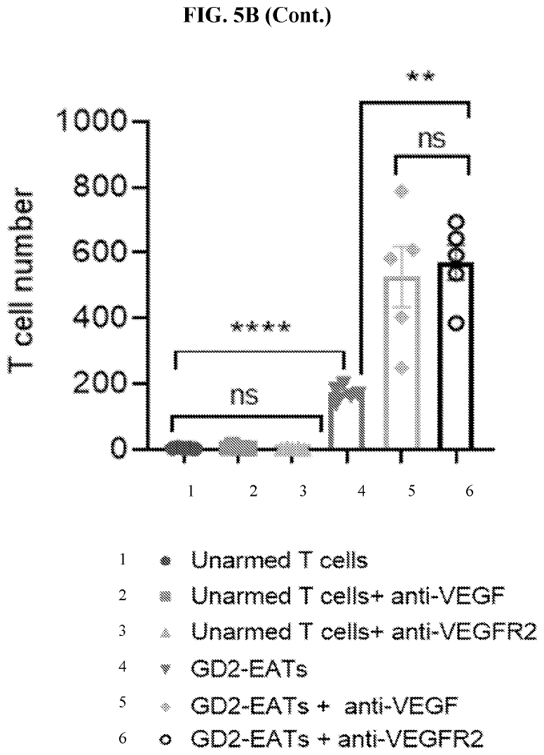

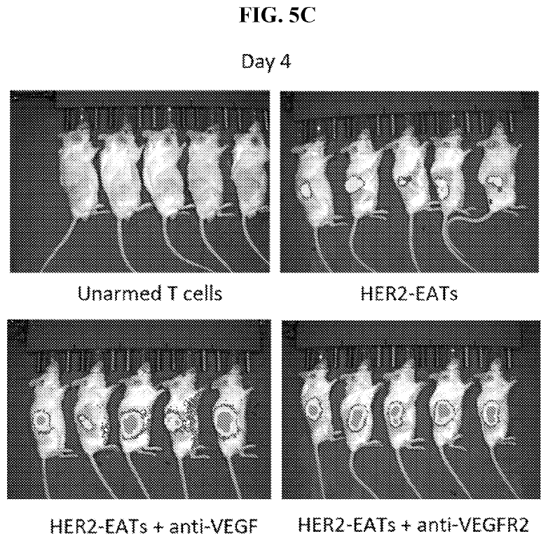

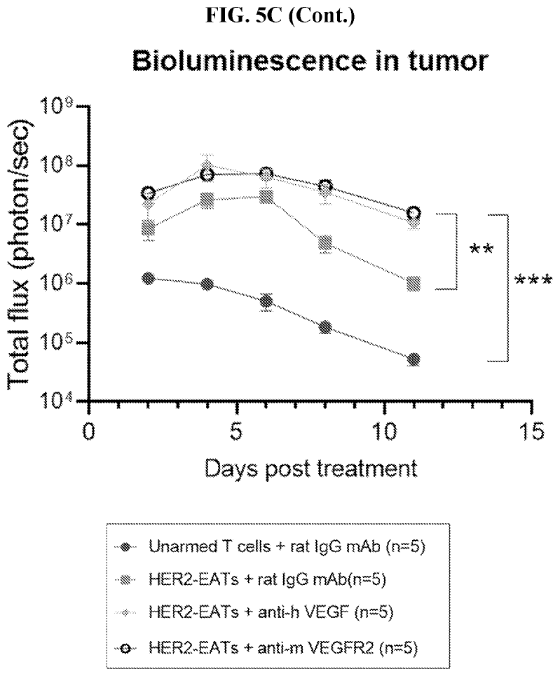

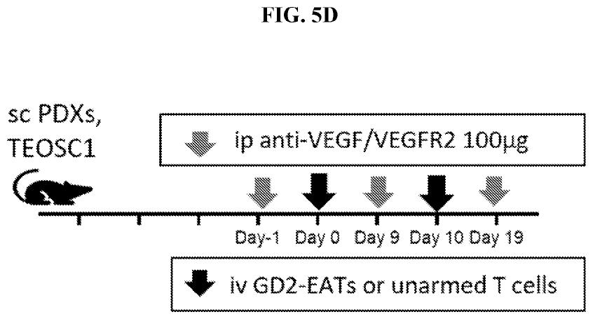

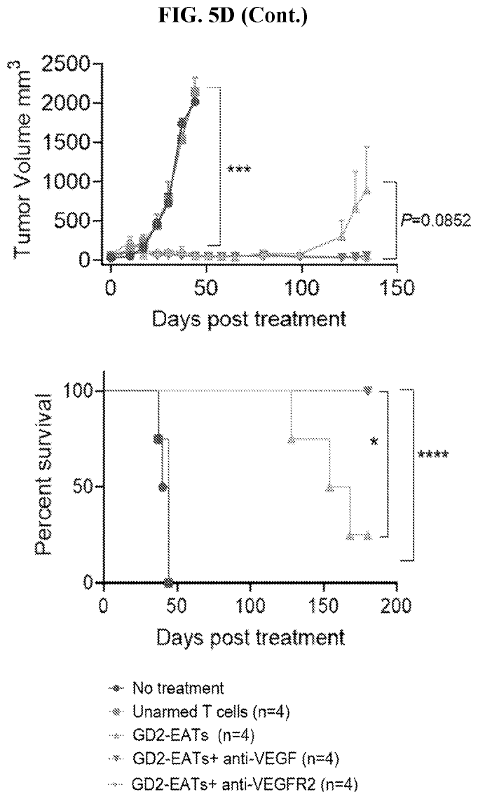

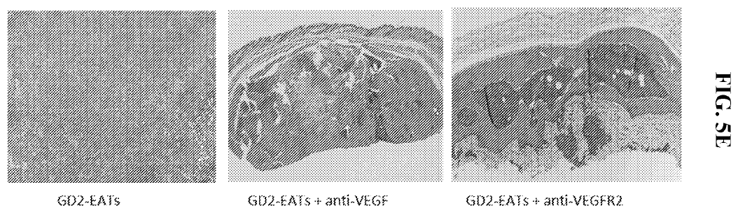

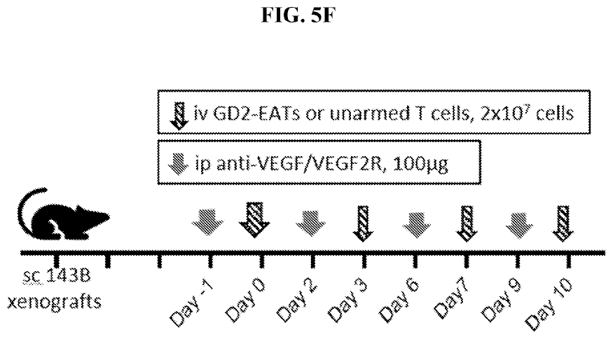

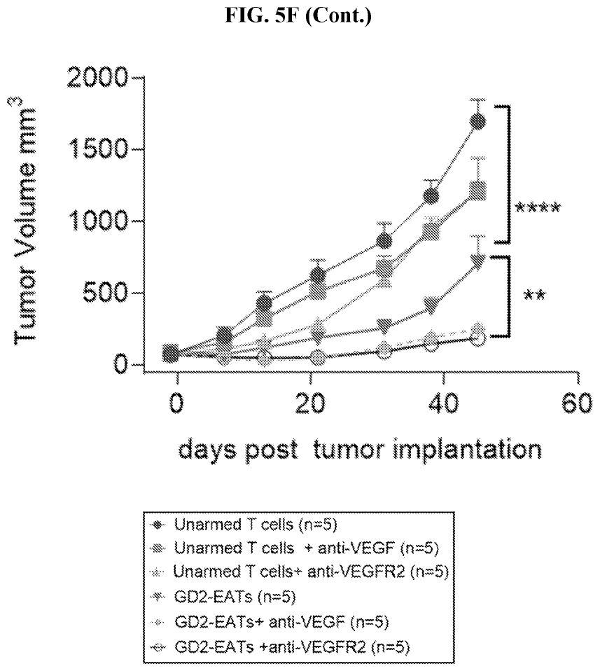

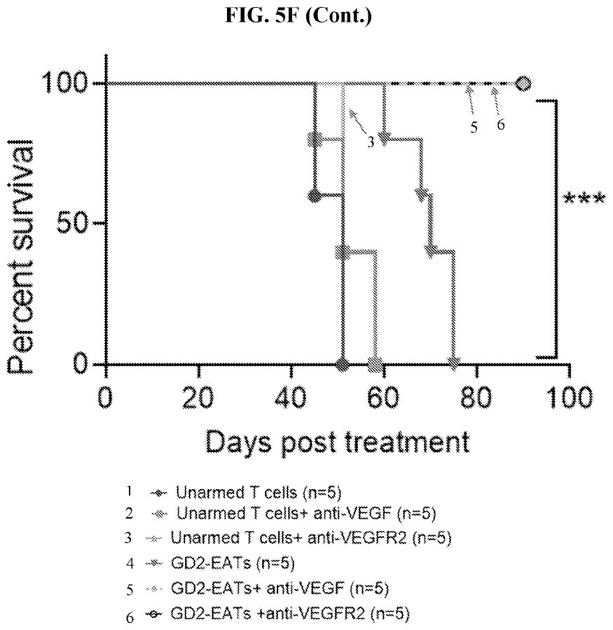

To target VEGF signaling pathway, anti-VEGF2 antibody (bevacizumab, anti-VEGF) or anti-VEGF receptor-2 antibody (anti-VEGFR2) was combined with GD2-BsAb treatment. 100 μg of each antibody was administered prior to unarmed T cells or GD2-EATs to treat osteosarcoma 143B xenografts. Flow cytometry analysis of tumors harvested on day 60 showed increased frequencies of tumor infiltrating hCD45(+) T cells and CD8(+) T cells when anti-VEGF or anti-VEGFR2 antibody was added to GD2-EAT treatment (FIG. 5A). Enhanced T cell infiltration in tumors after VEGF blockades was confirmed by IHC staining using anti-human CD3 antibody (FIG. 5B): both combinations of GD2-EATs with anti-VEGF or anti-VEGFR2 antibodies significantly increased T cell count in the tumors compared to GD2-EATs (P=0.0051 and P<0.0001, respectively).

The effect of VEGF blockades on T cell trafficking into tumors was studied using HER2 (+) osteosarcoma HGSOS PDX mouse model (FIG. 5C). 1×10⁷ of Luc(+) HER2-EATs were administered with or without ip anti-VEGF or anti-VEGFR2, and the bioluminescence intensity was monitored. The signal intensities significantly increased with VEGF blockades. HER2-EATs successfully trafficked into tumors, and the bioluminescence peaked on day 6 to 8 and then rapidly decreased. VEGF blockades significantly increased the T cell bioluminescence in tumors and enhanced persistence of HER2-EATs in tumors. The tumors treated with HER2-EATs plus anti-VEGF or anti-VEGFR2 maintained high bioluminescence for more than 14 days.

The in vivo effect of VEGF blockades on anti-tumor response of BsAb-directed T cell immunotherapy was evaluated in osteosarcoma 143B cell line xenograft and TEOSC1 PDX models. For TEOSC1 PDXs, 2 doses of GD2-EATs were given with 3 doses of either anti-VEGF or anti-VEGFR2 antibody (FIG. 5D). The combination with VEGF blockades produced long-term remission; mice treated with GD2-EATs plus VEGF blockades showed durable remission past 6 months and improved survival over GD2-EATs alone (P=0.0221). Interestingly, the tumors treated with VEGF blockade combinations showed fibrous and cavitary bone lesions characterized by intracavitary extramedullary hematopoiesis and mineralization, in contrast to recurred tumors treated with GD2-EATs alone showing chondroid differentiating osteosarcoma with T cell infiltration (FIG. 5E). When the effect of targeting VEGF/VEGFR2 was tested in osteosarcoma 143B xenograft model (FIG. 5F), both antibodies significantly enhanced anti-tumor response of GD2-EATs (P=0.0083 and P=0.0073), leading to improved tumor control and survival (P=0.0002).

### Example 7: Dexamethasone Premedication to Modulate Anti-Tumor Immunity

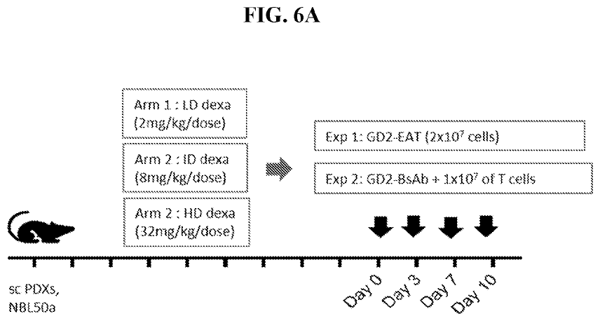

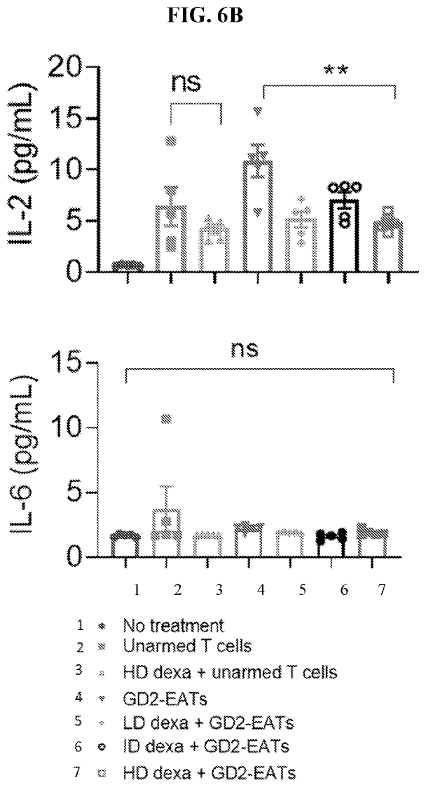

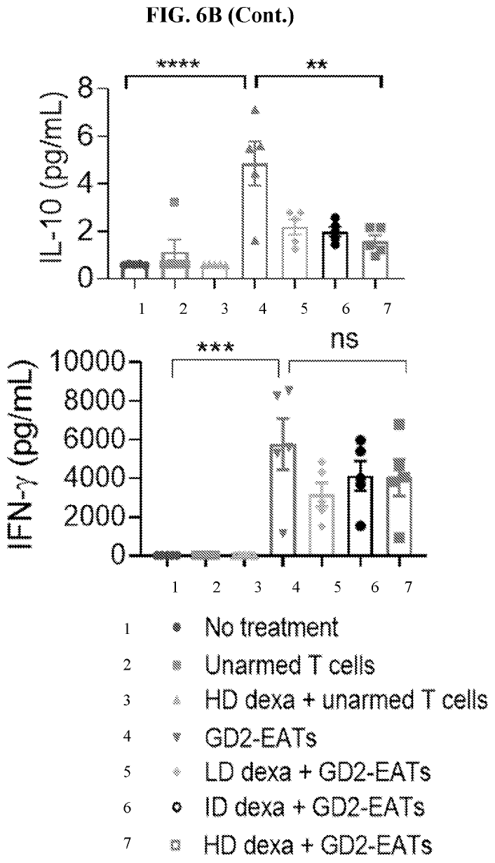

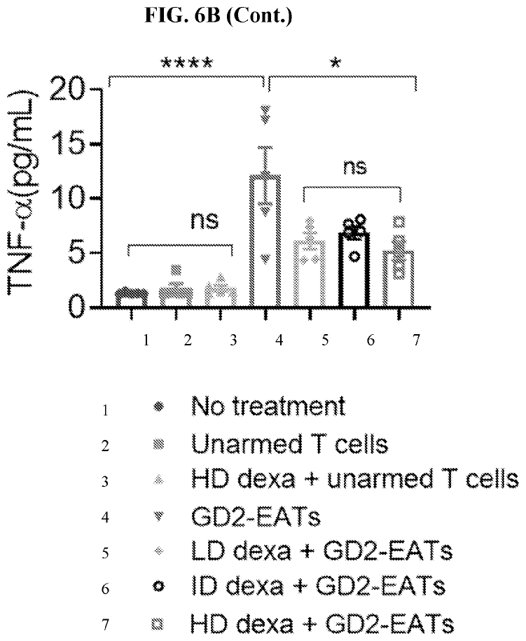

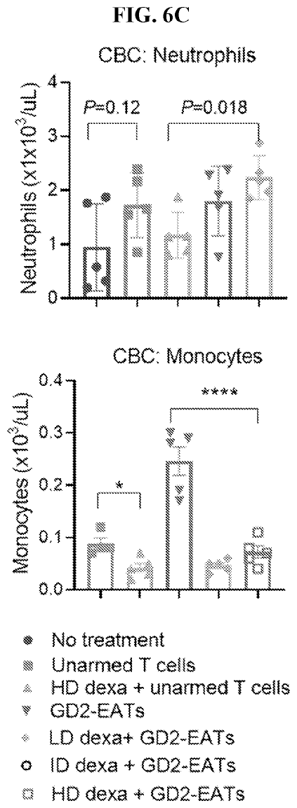

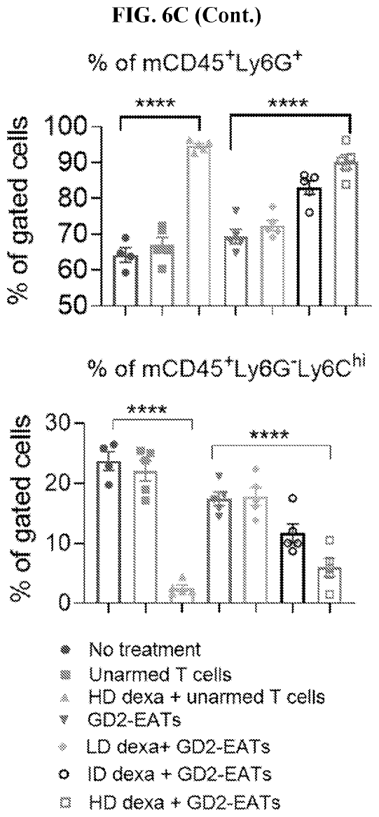

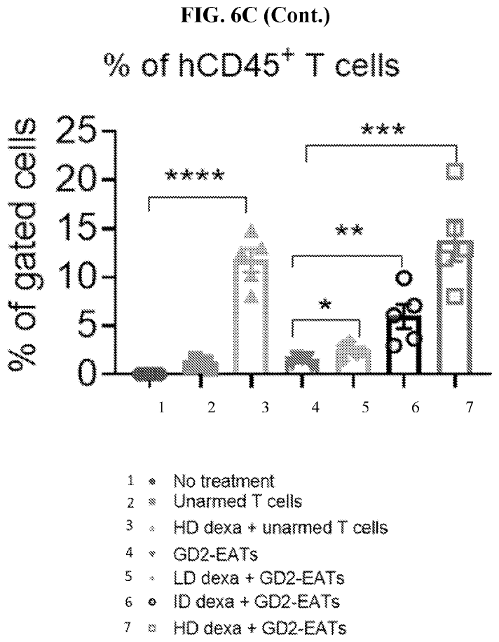

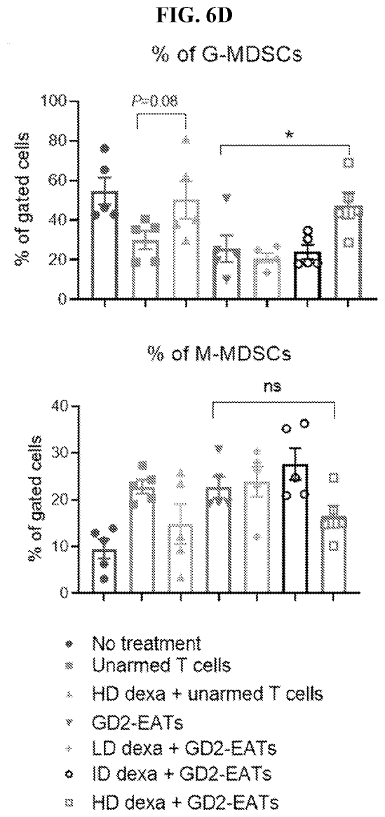

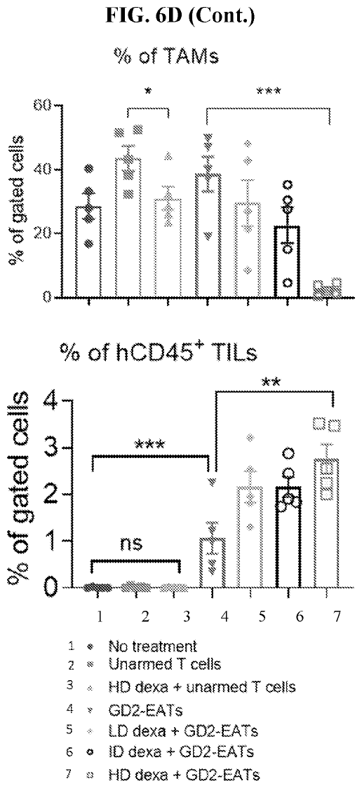

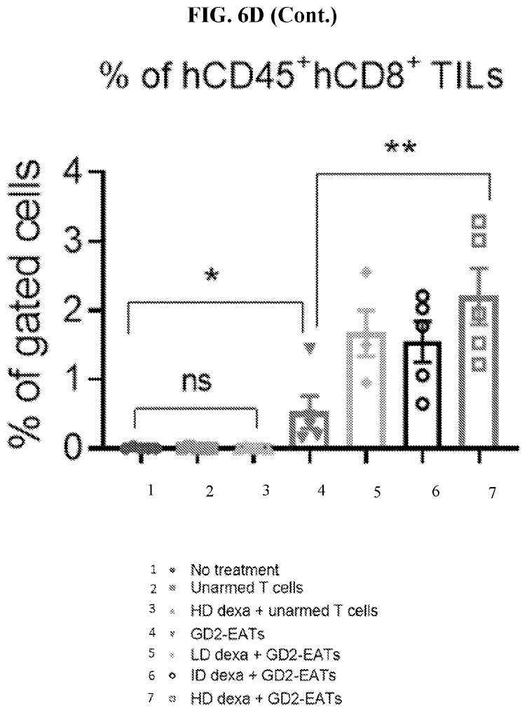

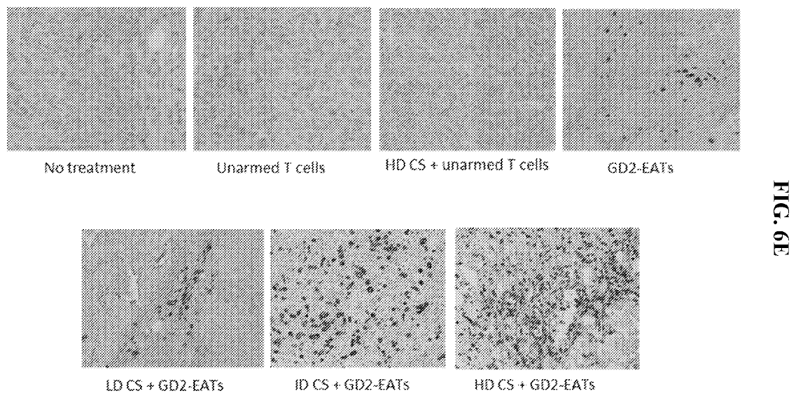

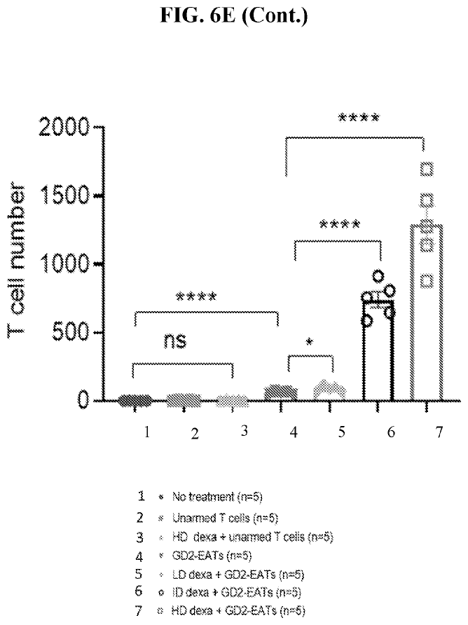

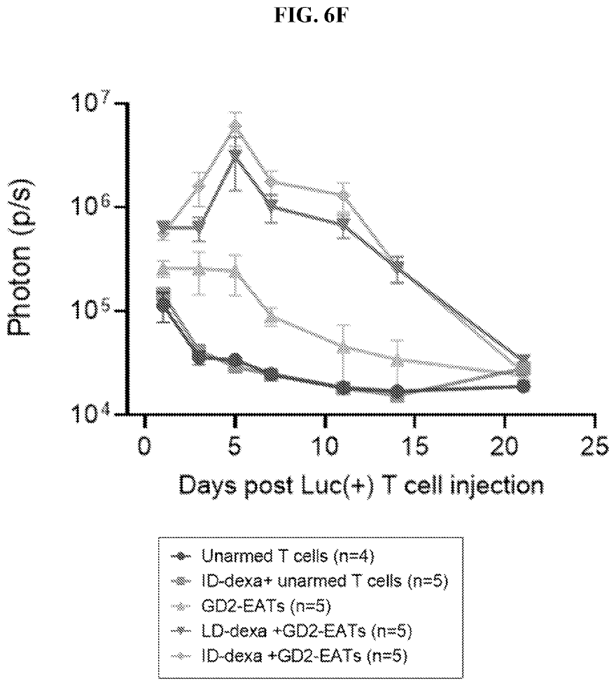

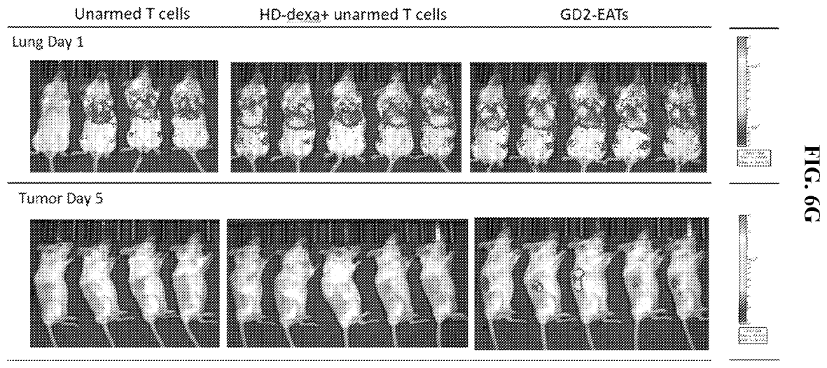

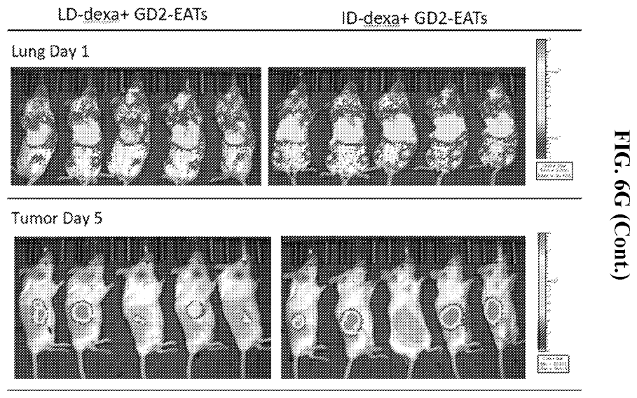

Corticosteroid premedication is routinely given to mitigate cytokine release following BsAb treatment. The effects of dexamethasone on the immune response of BsAb treatment were tested. The effect of dexamethasone on tumor infiltrating myeloid cells and lymphocytes driven by BsAb was evaluated, and the effect on in vivo anti-tumor response was assessed. 3 increasing dose levels of dexamethasone [low-dose (LD); 2 mg/kg/dose, intermediate-dose (ID); 8 mg/kg/dose, and high-dose (HD); 32 mg/kg/dose] were tested as premedication administered ip 1 hour before each GD2-EATs or GD2-BsAb injection (FIG. 6A). Dexamethasone premedication decreased TNF-α release from GD2-EATs, while IFN-γ level was not significantly affected (FIG. 6B). Mice treated with dexamethasone showed neutrophilia and profound monocytopenia and had significantly higher frequencies of circulating T cells than mice treated without dexamethasone premedication (FIG. 6C). Dexamethasone also significantly affected the frequencies of TIMs and TILs (FIG. 6D). While the frequencies of G-MDSCs (mCD45+mCD11b⁺Ly6G⁺Ly6C^{lo}F4/80⁻) or M-MDSCs (mCD45+mCD11b+Ly6G−Ly6C^{hi}) did not change significantly compared to unarmed T cells or GD2-EATs alone, the frequencies of F4/80(+) TAMs (mCD45⁺Ly6G⁻Ly6C^{lo}F4/80⁺] significantly decreased by dexamethasone treatment (P=0.0014). The depletion of TAMs was dose-dependent, being most profound with HD dexamethasone (vs. no dexamethasone), P=0.0002; vs. LD, P=0.0057; vs. ID, P=0.0074). Depletion of TAMs was inversely correlated with the frequencies of TILs. Dexamethasone increased the frequency of hCD45(+) TILs, and especially CD8(+) T cells in a dose-dependent fashion. HD dexamethasone had the greatest frequencies of hCD8(+) TILs among groups. This effect of dexamethasone on BsAb-driven T cell infiltration in tumors was confirmed by IHC staining using anti-human CD3 antibody (FIG. 6E). HD dexamethasone group showed significantly more TILs compared to GD2-EATs alone (P<0.0001).

Dexamethasone premedication also influenced BsAb-driven T cell trafficking into tumors. Bioluminescence signals was monitored following Luc(+) GD2-EAT treatment in GD2(+) osteosarcoma PDX mouse model (FIG. 6F). Mice received dexamethasone premedication showed much stronger signal intensities than mice treated without dexamethasone (FIG. 6G). The mean signal intensity of GD2-EATs plus HD dexamethasone was more than 10 times stronger than GD2-EATs alone on day 5 and maintained higher on day 14.

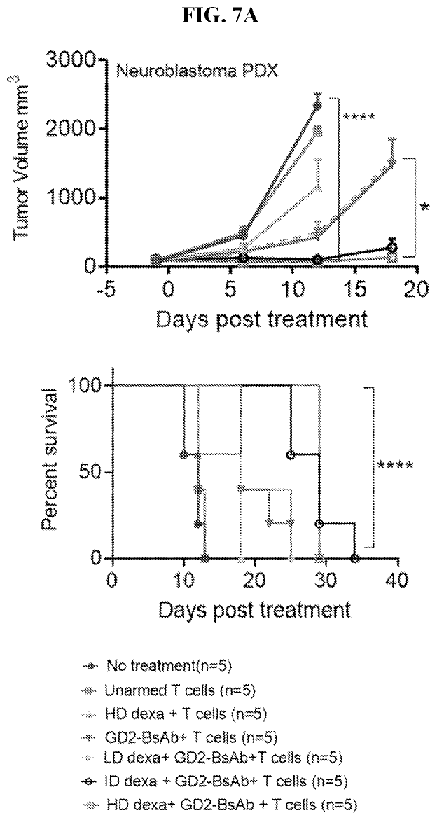

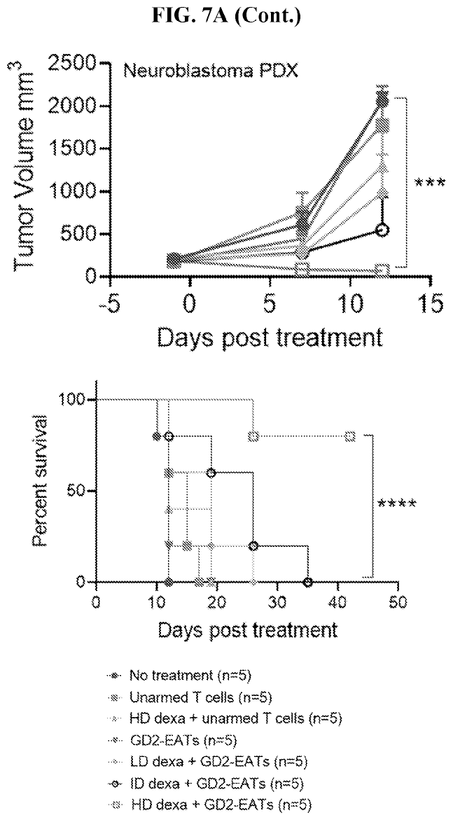

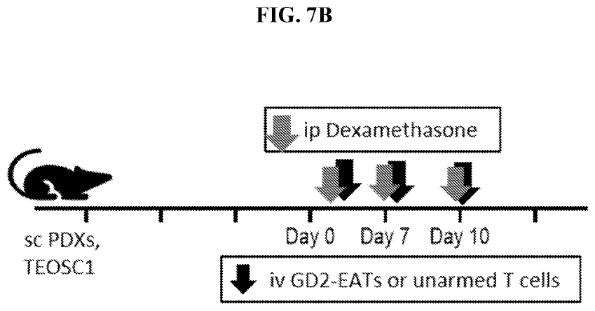

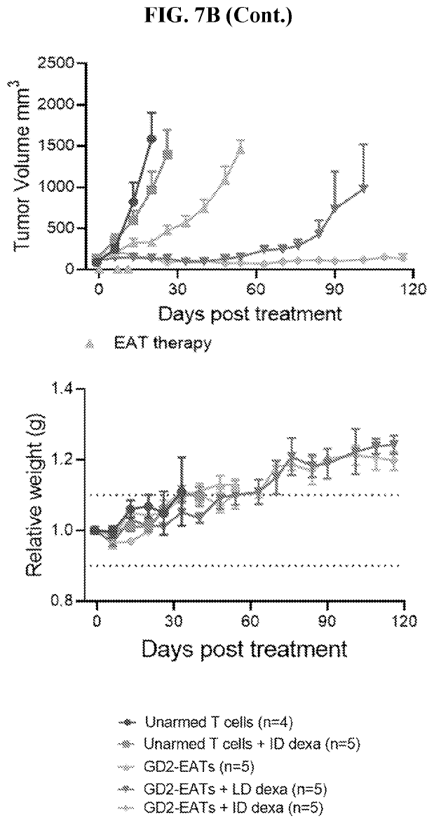

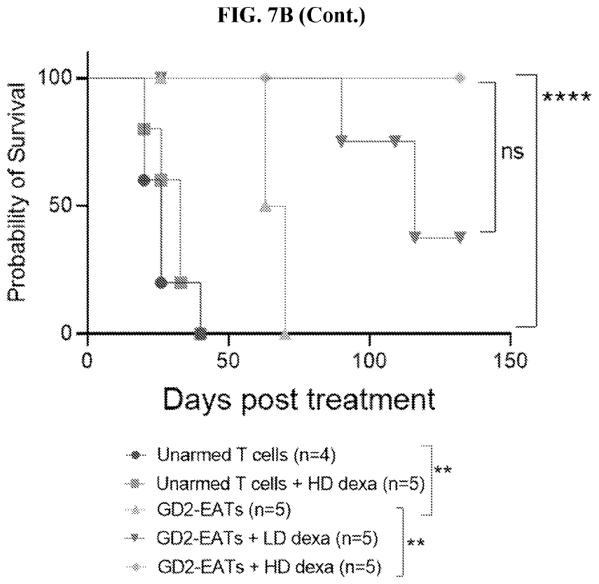

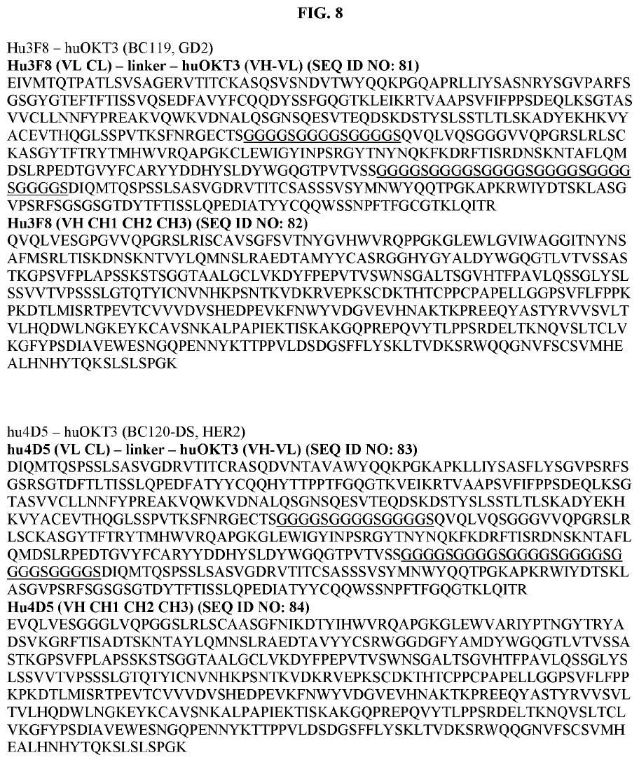

In vivo anti-tumor response correlated with the frequencies of T cell in the circulation and tumors and bioluminescence signals in tumors. When the anti-tumor responses among groups were compared (FIG. 6A), dexamethasone did not compromise anti-tumor effect of GD2-BsAb against neuroblastoma PDXs (FIG. 7A). Rather, ID and HD dexamethasone enhanced anti-tumor effect of GD2-BsAb against neuroblastoma PDXs when administered either as iv BsAb+T cells or as EATs. The addition of ID and HD dexamethasone improved tumor control and significantly improved survival (P<0.0001). This tumor suppressive role of dexamethasone on GD2-EATs was confirmed in another GD2(+) osteosarcoma PDX mouse model. LD or HD dexamethasone was administered before GD2-EATs, and the dexamethasone significantly improved tumor control and prolonged survival in a dose-dependent manner (FIG. 7B).

### Example 8: Potentiation of Anti-Tumor Effect of Anti-HER2 T Cell Encasing Bispecific Antibody Against Breast Cancer Using Dexamethasone

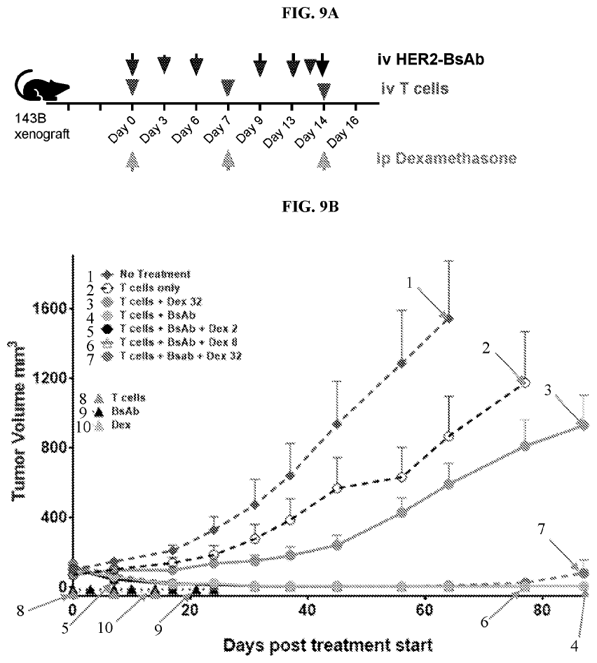

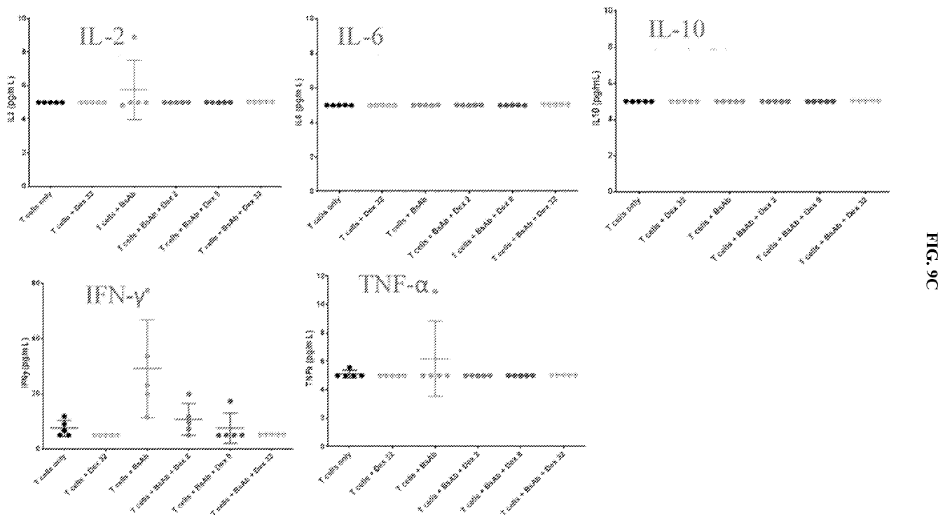

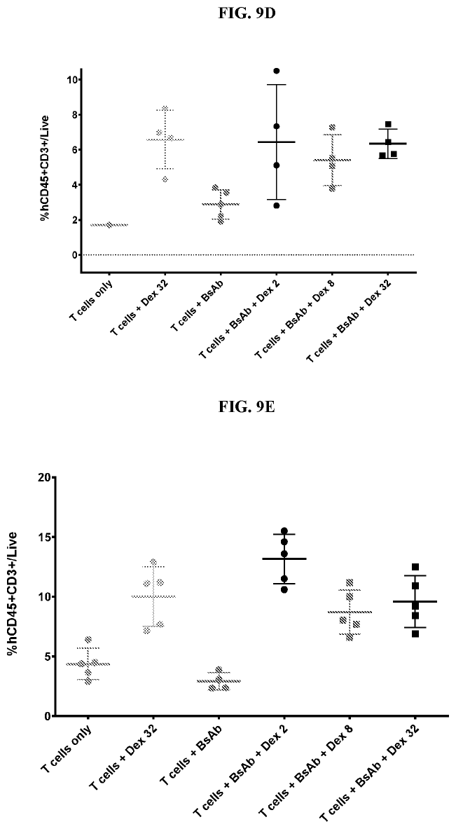

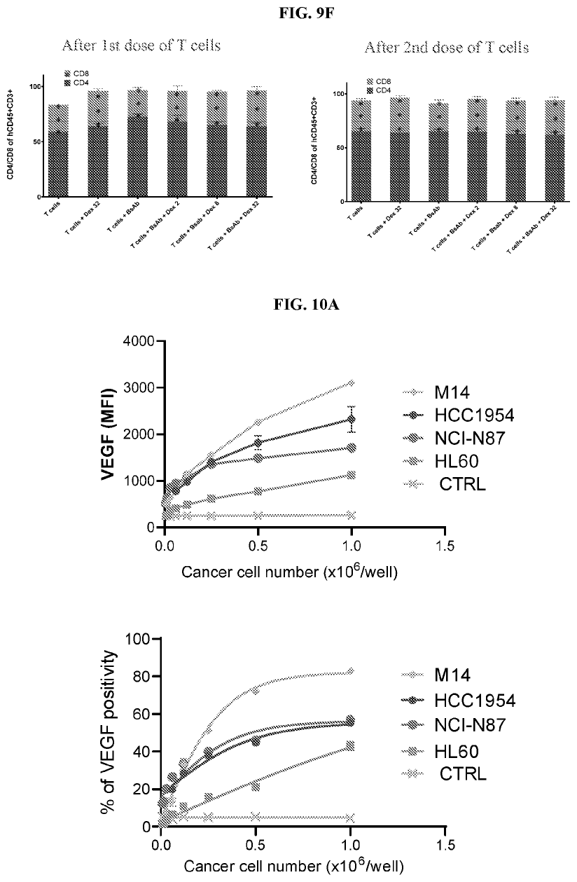

Increasing dosage of dexamethasone was tested as a premedication administered i.p. before the i.v. injection of HER2×CD3-BsAb plus activated human T cells in mice xenografted s.c. with M37 breast cancer PDX using the schedule detailed in FIG. 9A. The three dosages of intraperitoneal dexamethasone were 2 mg/kg (Dex 2), 8 mg/kg (Dex 8) and 32 mg/kg (Dex 32) once a week×3 weeks.

The in vivo anti-tumor effect is summarized in FIG. 9B, showing the strong anti-tumor response of the HER2×CD3-BsAb was not interfered by dexamethasone irrespective of dosage used, although at 32 mg/kg of dexamethasone, tumor escape occurred in one of the 5 mice. However, the mice treated with low and intermediate dosage (but not high dosage) of dexamethasone suffered weight loss around day 30 post treatment, primarily due to diarrhea from C. difficile bacteria infection.

In vivo serum cytokine levels were measured at 16 hours post treatment and compared among treatment groups (FIG. 9C). Although there was a general trend of decrease in IL2, IFN-gamma and TNF-alpha when dexamethasone was used, the differences were not statistically significant, most likely because of the small number of mice per cage.

Peripheral blood samples were also analyzed to compare the T cell engraftment. Human CD45(+)CD3(+) T cells were analyzed by flow cytometry after 1st dose of T cells (FIG. 9D) and 2nd dose of T cells (FIG. 9E), respectively. Dexamethasone treatment significantly increased circulating T cell frequencies after T cells injection with or without HER2-BsAb, most striking after the 2nd dose of T cell injection (FIG. 9E) (p<0.01). There was no difference in the CD4 to CD8 ratio among T cells among different treatment groups (FIG. 9F).

### Example 9: Combination Therapy with VEGF/VEGFR Blockade and Tumor-Specific T Cell Encasing BsAbs

### Experimental Methods

Tumor cell lines. Representative melanoma cell line M14 (NCI-DTP Cat #M14, RRID:CVCL_1395), small cell lung cancer cell line NCI-N87 (ATCC Cat #CRL-5822, RRID:CVCL_1603), human leukemia cell line HL6 (ATCC Cat #CCL-240, RRID:CVCL_0002), breast cancer cell line HCC1954 (ATCC Cat #CRL-2338, RRID:CVCL_1259), osteosarcoma cell line 143B (ATCC Cat #CRL-8303, RRID:CVCL_2270), and hepatoblastoma cell line Hep-G2 (ATCC Cat #HB-8065, RRID:CVCL_0027) were purchased. All the cell lines used were authenticated by short tandem repeat profiling with PowerPlex 1.2 System (Promega), and periodically tested for mycoplasma infection using MycoAlert™ PLUS Mycoplasma Detection Kit (Lonza). The cells were cultured in RPMI1640 (Sigma) supplemented with 10% heat-inactivated fetal bovine serum (FBS, Life Technologies) at 37° C. in a 5% CO₂ humidified incubator. The luciferase-labeled melanoma cell line M14Luc were generated by retroviral infection with an SFG-GF Luc vector.

Antibodies. To target VEGF, the anti-human VEGF (hVEGF) antibody bevacizumab (R and D Systems Cat #MAB9947, RRID:AB_2892608) and anti-mouse VEGFR2 (mVEGFR2) antibody DC101 (Bio X Cell Cat #BE0060, RRID:AB_1107766) were used. The VEGF inhibitors were given intraperitoneally (ip) one day before each EAT injection. Anti-GD2, anti-HER2, and anti-glypican3 (GPC3) BsAbs were mainly used in the experiments to test the synergy between EATs and VEGF inhibitors. These BsAbs were built on the IgG-[L]-scFv format using the sequences of anti-CD3 (huOKT3) IgG and anti-GD2 hu3F8 IgG1, anti-epidermal growth factor receptor-2 (HER2) (trastuzumab) IgG1, or anti-GPC3 antibody (clone GC33). For each BsAb, scFv of huOKT3 was fused to the C-terminus of the light chain of human IgG1 via a C-terminal (G₄S)₃ linker (SEQ ID NO: 154). N297A and K322A on Fc were generated with site-directed mutagenesis via primer extension in polymerase chain reactions. The nucleotide sequence encoding each BsAb was synthesized by GenScript and was subcloned into a mammalian expression vector. BsAb was produced using Expi293™ expression system (Thermo Fisher Scientific) separately. Antibodies were purified with protein A affinity column chromatography. The purity of these antibodies was tested by size-exclusion high-performance liquid chromatography (SE-HPLC).

T cell expansion ex vivo. Serially expanded T cells from a single donor were used for each individual experiment. Peripheral blood mononuclear cells (PBMCs) were separated from buffy coats (New York Blood Center) by Ficoll. Naïve T cells were purified using Pan T cell isolation kit (Miltenyi Biotec, Cat #130096535) and expanded by CD3/CD28 Dynabeads (Gibco™, Cat #11132D) for 7 to 14 days in the presence of 30 IU/mL of IL-2. Expanded T cells were analyzed for their proportion of CD3(+), CD4(+), and CD8(+) T cells, and the fraction of CD4(+) or CD8(+) T cells was allowed between 40% and 60% to maintain consistency. Unless stated otherwise, these activated T cells were used for all T cell experiments.

Flow cytometry analysis of VEGF expression. Increasing numbers of various cancer cells (4×10³ to 1×10⁶ cells/well), including gastric cancer cell line NCI-N87, melanoma cell line M14Luc, leukemia cell line HL60, and breast cancer cell line HCC1954, were incubated in 96-well plates at 37° C. in a 5% CO₂ humidified incubator. v Cancer cells were harvested after 48 hours and stained with anti-hVEGF APC-conjugated antibody (R and D Systems Cat #IC2931A, RRID:AB_357310) according to manufacturer's instructions. 1×10⁶ of cancer cells were incubated with the antibody at 4° C. for 30 minutes, washed, and analyzed by flow cytometry (BD FACS Calibur instrument and Attune NxT Flow Cytometer). Palivizumab was used as a control antibody. Data were analyzed with FlowJo V10 software (FlowJo, RRID:SCR_008520).

Measurement of serum VEGF levels. Serum VEGF levels in murine plasma were measured using Quantikine enzyme-linked immunosorbent assay (ELISA) kit (R and D Systems Cat #DVE00, RRID:AB_2800364) according to the manufacturer's instruction. The intensity of the reaction was then revealed with tetramethylbenzidine and optical density was measured at 450 nm and at 540 nm using a Vmax microplate reader and Soft MAX Pro software (Molecular Devices, Menlo Park, CA, USA). All samples were run in triplicate, and a standard curve was established for each assay.

Therapeutic study with mouse xenograft model All animal procedures were performed in compliance with Memorial Sloan Kettering Cancer Center's institutional Animal Care and Use Committee (IACUC) guidelines. BALB/c Rag2^{-/-}IL-2Rγc^{-/-} (BRG) mice purchased from Taconics Inc. were used in this study. Cancer cell line xenografts (CDXs) were established using 1×10⁶ of 143B and 5×10⁶ of Hep-G2 cell lines. Patient-derived xenografts (PDXs) were established from fresh surgical specimens with MSKCC IRB approval from patients diagnosed with neuroblastoma or osteoblastoma. Tumor cells in Matrigel (Corning Corp) were implanted subcutaneously on the right flank of each mouse. To avoid biological variables, only female mice were used for in vivo experiments. Treatment was initiated after tumors were established, average tumor volume of 100 mm³ when measured using TM900 scanner (Piera, Brussels, BE). Before treatment, mice with small tumors (<50 mm³) or infection signs were excluded from randomization to experimental groups. For ex vivo arming of T cells, 10 g of BsAb were used to arm 2×10⁷ of T cells per injection[22, 23]. The VEGF blockades (Bevacizumab and DC101) were given 100 g/dose intraperitoneal (ip) one day before each EAT injection. Tumor growth curves and overall survival were analyzed, and the overall survival was defined as the time from start of treatment to when tumor volume reached 2000 mm³. To define the well-being of mice, CBC analyses, changes in body weight, behavior and physical appearance were monitored. All animal experiments were repeated twice more with different donor's T cells to ensure that our results were reliable.

T cell transduction. T cells isolated from peripheral blood were stimulated with Dynabeads™ Human T-Activator CD3/CD28 for 24 hours. T cells were transduced with retroviral constructs containing tdTomato and click beetle red luciferase in RetroNectin-coated 6-well plates in the presence of IL-2 (100 IU/ml) and protamine sulfate (4 μg/mL). Transduced T cells were cultured for 8 days before being used in animal experiments.

Bioluminescence imaging. Mice were anesthetized and imaged after intravenous injection of 3 mg of D-luciferin (Gold Biotechnology) on different days post T cell injection. Images were acquired using IVIS Spectrum CT In vivo Imaging System (Caliper Life Sciences). Bioluminescence images were overlaid with photographs, and regions of interest (ROI) were drawn based on the location and contour of tumor using Living image 2.60 (Xenogen). The total counts of photons (photon/s) were obtained. Bioluminescence signals (total flux, photon/s) before T cell injection were used as baselines.

Immunohistochemistry and immunofluorescence staining. Immunohistochemistry (TIC) staining were performed at the MSK Molecular Cytology Core Facility using Discovery XT processor (Ventana Medical Systems). Tumor samples were fixed and embedded in paraffin. Anti-human CD45, anti-human CD3, anti-human CD4, anti-human CD8, anti-mouse CD31 antibodies were used, which was followed by biotinylated secondary antibody. The detection was performed using a DAB detection kit (Ventana Medical Systems) or Alexa Fluor™ 488 or 568 Tyramide Reagent (Invitrogen). IHC images were captured from tumor sections using a Nikon ECLIPSE Ni-U microscope and NIS-Elements 4.0 imaging software. Antigen positive cells were counted with Qupath 0.1.2 or using positive pixel count analysis.

Positive Pixel Count Analysis. IHC slides were scanned (Aperio ScanScope XT) and analyzed by comparing positive pixel counts (Aperio Technologies). For analyzing tumor infiltrating lymphocytes, the largest area of intact tumor tissue was included, and oblique sections were avoided. Each slide was visually inspected to ensure specificity and sensitivity of antibody staining. After positive pixel count analysis was run, analyzed slides were examined to confirm that positively identified pixels were consistent with lymphocyte staining and not background staining. Percentages were calculated as the total number of positive pixels divided by the total number of pixels (% positive pixels/total pixels).

Flow cytometry analysis. For flow cytometry analyses of blood and tumor samples from mice, the following antibodies were purchased from Biolegend: anti-human CD45-APC (HI30), anti-human CD3-Percp/Cy5.5 (SK7), anti-human CD8-FITC, anti-human CD4-PE/Cy7, anti-mouse CD45-Brilliant Violet 711™ (30-F11), anti-mouse CD11b-Brilliant Violet 570™ (M1/70), anti-mouse Ly6G-FITC, anti-mouse Ly6C-PerCP/Cy5.5, and anti-mouse F4/80-PE.

Cytokine release assays. EAT-induced human cytokine release was analyzed in vitro and in vivo. Human Th1 cell released cytokines were analyzed by LEGENDplex™ Human Th1 Panel (Biolegend, Cat #741035). Five T cell cytokines including IL-2, IL-6, IL-10, IFN-γ and TNF-α were analyzed using mouse serum after treatment with EATs with or without VEGF blockade.

Statistical analysis. In vivo anti-tumor effect was compared by tumor growth curves and survival curves. Area under curves (AUCs) of tumor growth were calculated and compared among groups. Differences between samples indicated in the figures were tested for statistical significance by two-tailed Student's t-test for two sets of data while one-way ANOVA with Tukey's post hoc test was used to among three or more sets of data. All statistical analyses were performed using GraphPad Prism V.8.0 for Windows (GraphPad Software, La Jolla, CA, www.graphpad.com). P value <0.05 was considered statistically significant. Asterisks indicate that the experimental P value is statistically significantly different from the associated controls at *P<0.05; **P<0.01; ***P<0.001, ****P<0.0001.

### VEGF Expression on Tumor Cell Increased by Increasing Density of Cancer Cells

Flow cytometry analyses revealed an increase in VEGF expression when seeding density was increased (FIG. 10A). Tumor cells were stained with anti-human VEGF antibody, and the MFIs of the cell lines M14Luc, HCC1954, NCI-N87, and HL60 incubated in high densities were 2- to 10-fold higher than those of the cancer cells in the lowest density (4×10³ cells/well). Particularly, M14Luc cell lines incubated in the wells of high cell density expressed significantly elevated levels of VEGF, while those in a low density did not express VEGF. The frequencies of VEGF positivity were also increased with the cell density. While a few cancer cells express VEGF when incubated in the lowest density, more than 50% of cancer cells express VEGF in the highest cell density.

### Anti-VEGF Antibody Reduced Serum VEGF Levels

Mice bearing HGSOC1 osteosarcoma PDX were treated with T cells ex vivo armed with anti-HER2-BsAb (HER2-EATs) with or without VEGF blockade using bevacizumab or DC101. These antibodies have no known species crossreactivities (Table 2).

TABLE 2
Cross interactions between human and
murine VEGF ligands and receptors

| | | | Bevacizumab | DC101 |
|---|---|---|---|---|
| | | | Effect | Effect |
| Ligand | Receptor | Interaction | expected | expected |
| hVEGF | hVEGFR | strong | Yes | No |
| hVEGF | mVEGFR | weak/intermediate | Yes | Yes |
| mVEGF | mVEGFR | strong | No | Yes |
| mVEGF | hVEGFR | weak | No | No |

After 2 doses of each treatment (day 5 post-treatment, at 48 hours after 2^{nd} dose of bevacizumab or DC101), serum h-VEGF levels were measured using Quantikine ELISA assay. In contrast to the DC101 not affecting the h-VEGF levels in the mouse blood, bevacizumab significantly reduced serum h-VEGF levels (FIG. 10B).

Effect of VEGF Blockade on Cytokine Levels after EAT Therapy

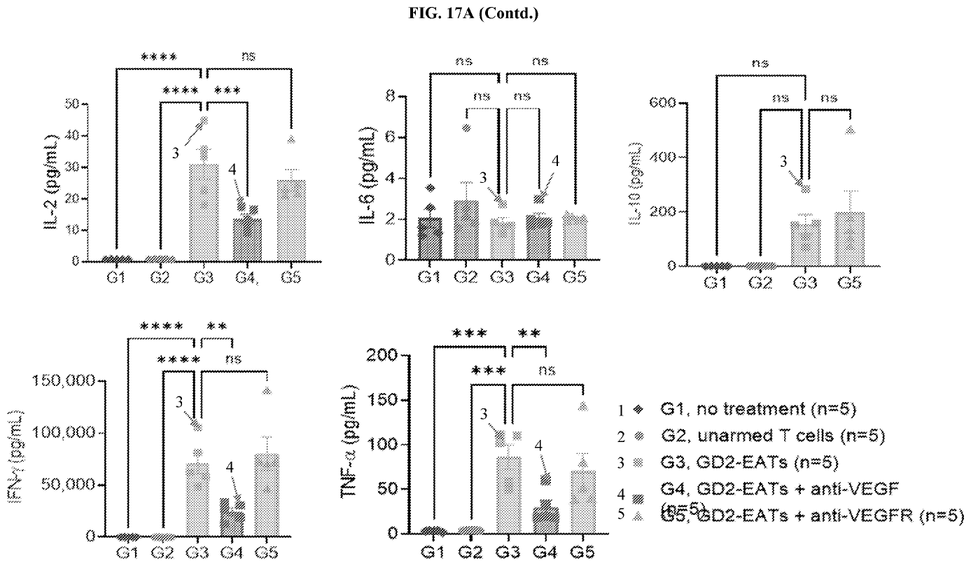

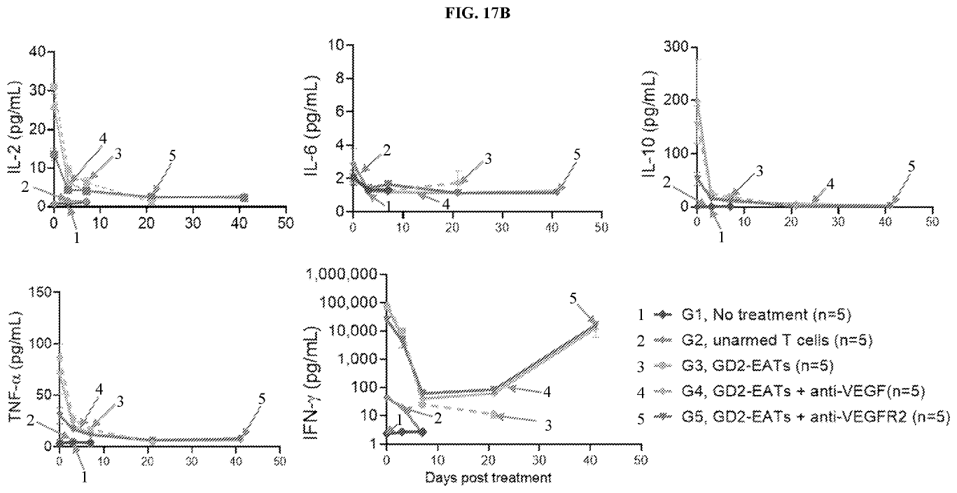

To investigate the effect of VEGF blockades on cytokine release by T cells, TH1 cell cytokine levels were measured after GD2-EATs injection following bevacizumab or DC101 in mice carrying neuroblastoma PDX (FIG. 17A). The TH1 cell cytokines (IL-2, IFN-γ, and TNF-α) 2 hours after the first dose of GD2-EATs were significantly lower in bevacizumab combination group, but not in DC101 combination group. Thereafter, despite repeated GD2-EATs injections, TH1 cell cytokines decreased overtime. However, IFN-7 which levelled off after day 7 rapidly rose after day 21 through day 40 both in GD2-EATs plus bevacizumab and in GD2-EATs plus DC101 treatment groups (FIG. 17B), coinciding with the strong anti-tumor effects shown in both combination groups.

VEGF Blockades Facilitate BsAb-Driven T Cell Trafficking into Tumors

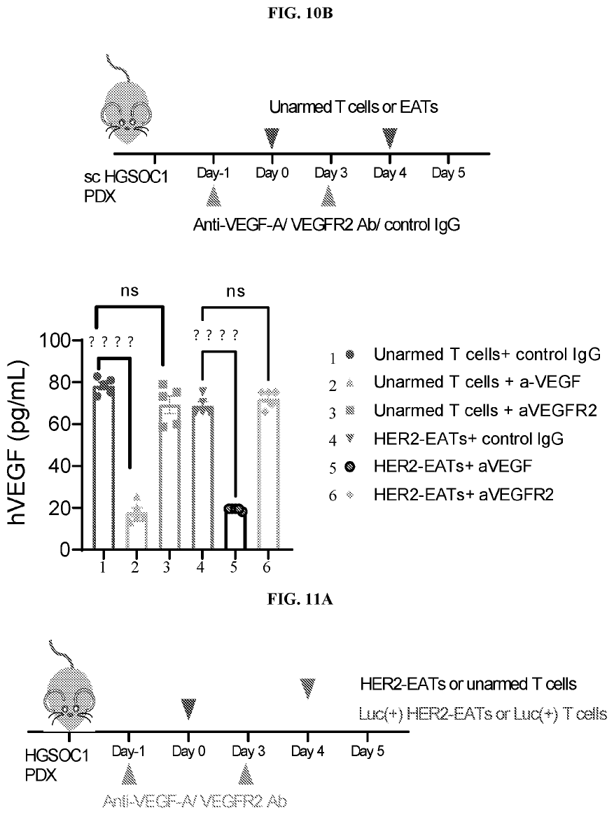

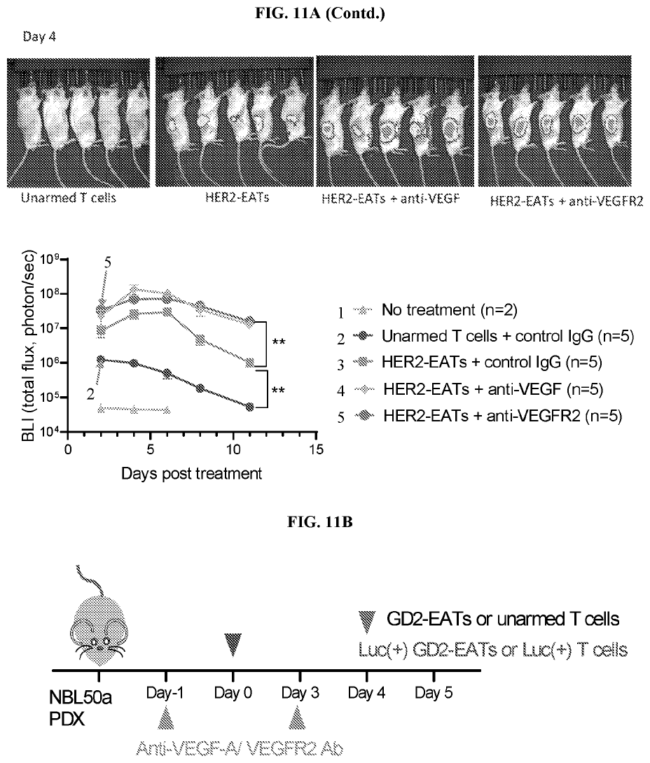

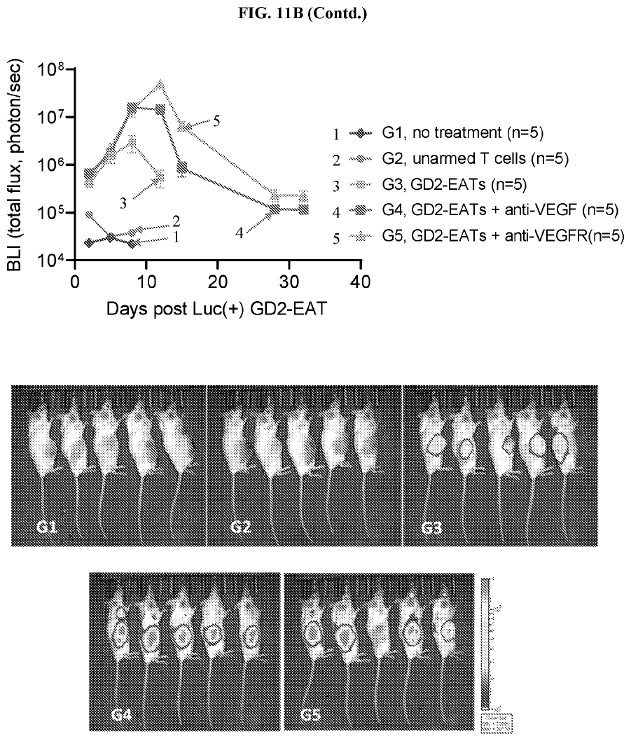

The effect of VEGF blockades on BsAb-driven T cell trafficking into solid tumors was investigated. Luciferase transduced T cells armed with anti-HER2-BsAb [Luc (+) HER2-EATs]were administered in combination with bevacizumab or with DC101 to mice bearing osteosarcoma PDX HGSOC1 (FIG. 11A). The bioluminescence of tumor infiltrating lymphocytes (TILs) were monitored over time and quantified as a bioluminescence intensity (BLI). HER2-EATs successfully trafficked into tumors, where their bioluminescence peaked around day 5. Both bevacizumab and DC101 significantly increased the BLIs of TILs in tumors and enhanced their persistence (7.5-fold and 2.4-fold increase in BLI AUC, p-value 0.05 and 0.016, respectively) (Table 3).

TABLE 3
Bioluminescence intensities (BLIs) of tumor infiltrating
lymphocytes analyzed by area under curves

| | | Fold | | | |
|---|---|---|---|---|---|
| | | increase | | | |
| | | in BLI | P- | | |
| Control | TIM depletion treatment | (AUC-BLI) | value | Tumor model | FIG. |
| HER2-EATs | unarmed T cells | 0.04 | 0.02 | osteosarcoma PDX | FIG. 2A |
| HER2-EATs | GD2-EATs + anti-VEGF | 7.5 | 0.05 | osteosarcoma PDX | FIG. 2A |
| HER2-EATs | GD2-EATs + anti-VEGFR2 | 2.4 | 0.02 | osteosarcoma PDX | FIG. 2A |
| GD2-EATs | unarmed T cells | 0.004 | 0.01 | neuroblastoma PDX | FIG. 2B |
| GD2-EATs | GD2-EATs + anti-VEGF | 2.8 | 0.01 | neuroblastoma PDX | FIG. 2B |
| GD2-EATs | GD2-EATs + anti-VEGFR2 | 8.1 | 0.05 | neuroblastoma PDX | FIG. 2B |

The persistence of HER2-EATs was also significantly longer with VEGF blockades. The effect of VEGF blockades on BsAb-driven T cell trafficking was tested in a separate tumor model using GD-EATs and GD2(+) neuroblastoma PDX (FIG. 11B). Both bevacizumab and DC101 increased BLIs in tumors (2.8-fold and 8.1-fold increase in BLI-AUC, p-value 0.01 and 0.05, respectively), significantly prolonging TIL persistence; The TIL bioluminescence peaked around day 7 to day 14 and remained detectable after 30 days post-treatment (FIG. 18).

VEGF Blockades Significantly Increased T Cell Infiltration into Tumors

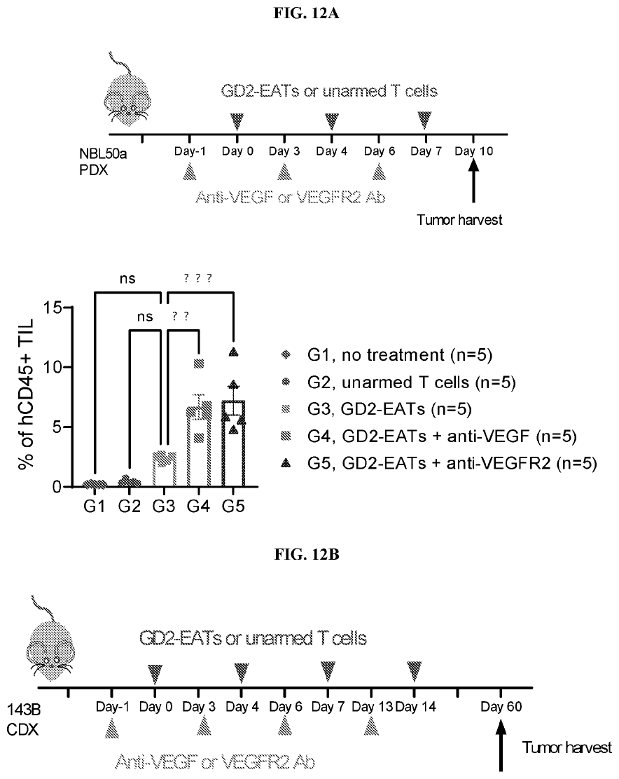

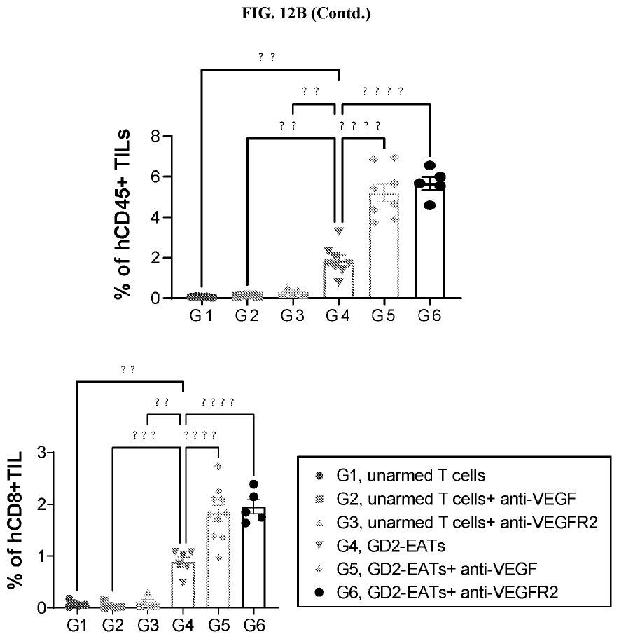

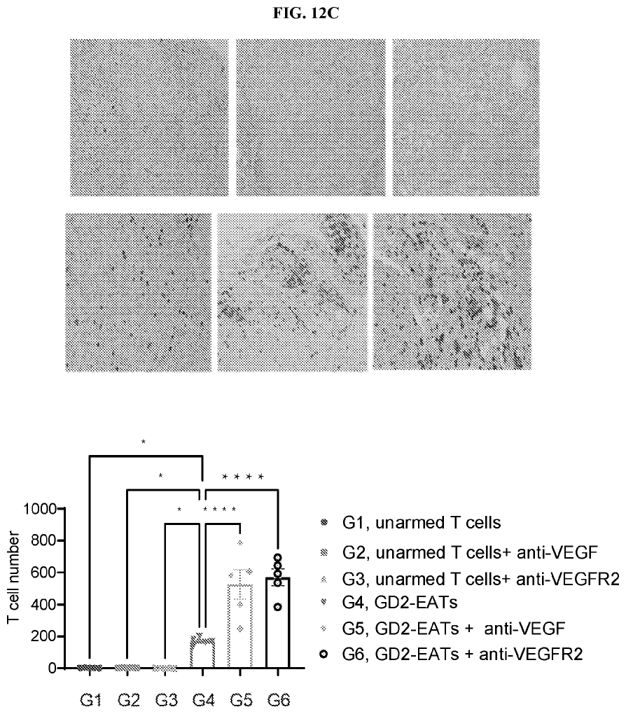

Neuroblastoma PDXs treated with GD2-EATs with or without VEGF blockade were analyzed by flow cytometry on day 10 post-treatment (FIG. 12A). Tumors treated with GD2-EATs showed higher frequencies of intratumoral hCD45(+) T cells than tumors treated with unarmed T cells. When bevacizumab or DC101 was combined with GD2-EATs, the increase of hCD45(+) TILs was much more pronounced. The enhanced intratumoral T cell infiltration by VEGF blockades was reproduced in a second model wherein 143B osteosarcoma CDXs were analyzed on day 60 post-treatment of GD2-EATs (FIG. 12B). In the presence of bevacizumab or DC101, not just hCD45(+) TILs but also hCD8(+) TILs were significantly increased.

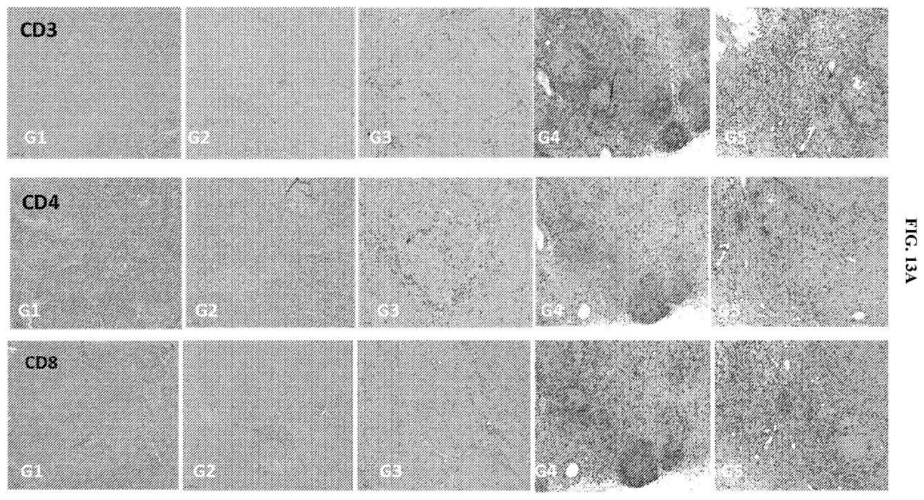

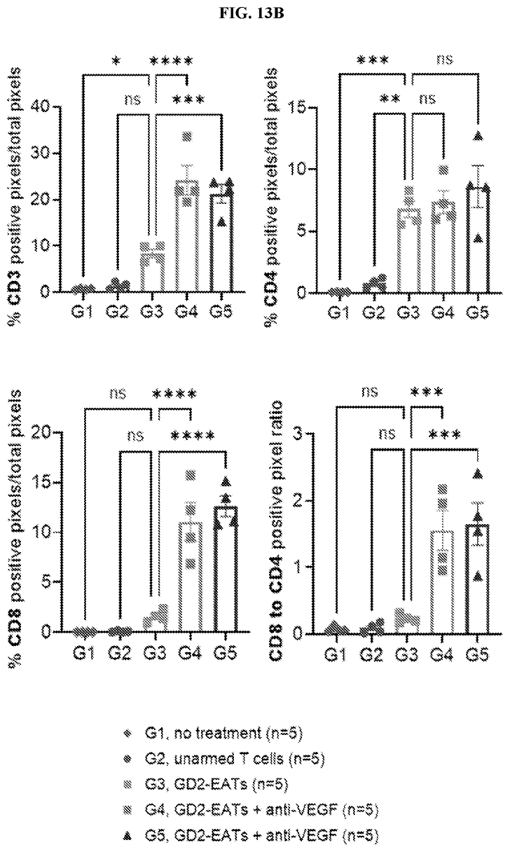

The effect of VEGF blockades on TILs was confirmed by IHC staining. In contrast to tumors treated by unarmed T cells with or without VEGF blockades showing few TILs, osteosarcoma CDXs treated by GD2-EATs showed diffuse T cell infiltration on day 60 post-treatment (FIG. 12C). By the intratumoral hCD3(+) T cell count, TILs were significantly increased by both bevacizumab and DC101 when compared to GD2-EATs alone (p<0.0001 and p<0.0001, respectively), with no difference between two blocking antibodies (P=0.69). CD3(+), CD4(+), or CD8(+) T cell infiltration in neuroblastoma PDX model were also compared (FIG. 13A). Tumors harvested on day 10 post-treatment showed a significant increase of TILs in the groups of GD2-EATs with VEGF blockades (FIG. 13B). Bevacizumab and DC101 showed a 3-fold and 2.5-fold increase of CD3(+) TILs compared to tumors treated by GD2-EATs alone, respectively. While CD4(+) TIL increase was only 1.1-fold and 1.3-fold increase, respectively, CD8(+) TIL increase was more robust, at 5-fold and 4-fold, respectively. Because of these shifts, the CD8/CD4 ratios underwent a significant increase after combined treatment of GD2-EATs with VEGF blockades (Table 4).

TABLE 4
Distribution of CD3, CD4, and CD8 T cells in neuroblastoma patient-derived
xenografts by positive pixel count analyses.

| | | P-value | | P-value | % CD8+ | P-value | Ratio of | P-value |
|---|---|---|---|---|---|---|---|---|
| | % CD3+ cells | (vs.GD2- | % CD4+ cells | (vs.GD2- | cells | (vs.GD2- | CD8 to CD4 | (vs.GD2- |
| Treatment | (mean ± SD) | EATs) | (mean ± SD) | EATs) | (mean ± SD) | EATs) | T cells | EATs) |
| Unarmed T | 1.4 ± 0.7 | 0.05 | 0.8 ± 0.4 | 0.001 | 0.07 ± 0.06 | 0.662 | 0.1 ± 0.07 | 0.96 |
| cells | | | | | | | | |
| GD2-EATs | 8.4 ± 2 | — | 6.8 ± 1.3 | — | 1.1 ± 0.3 | — | 0.2 ± 0.06 | — |
| GD2-EATs | 24 ± 6 | <0.0001 | 7.4 ± 1.8 | 0.98 | 5 ± 3 | <0.0001 | 1.6 ± 0.6 | 0.001 |
| +anti-VEGF | | | | | | | | |
| GD2-EATs | 21 ± 4 | 0 | 8.6 ± 3.4 | 0.45 | 4 ± 4 | <0.0001 | 1.7 ± 0.6 | 0.001 |
| +anti-VEGFR2 | | | | | | | | |

### VEGF Blockade and Tumor Infiltrating Myeloid Cells

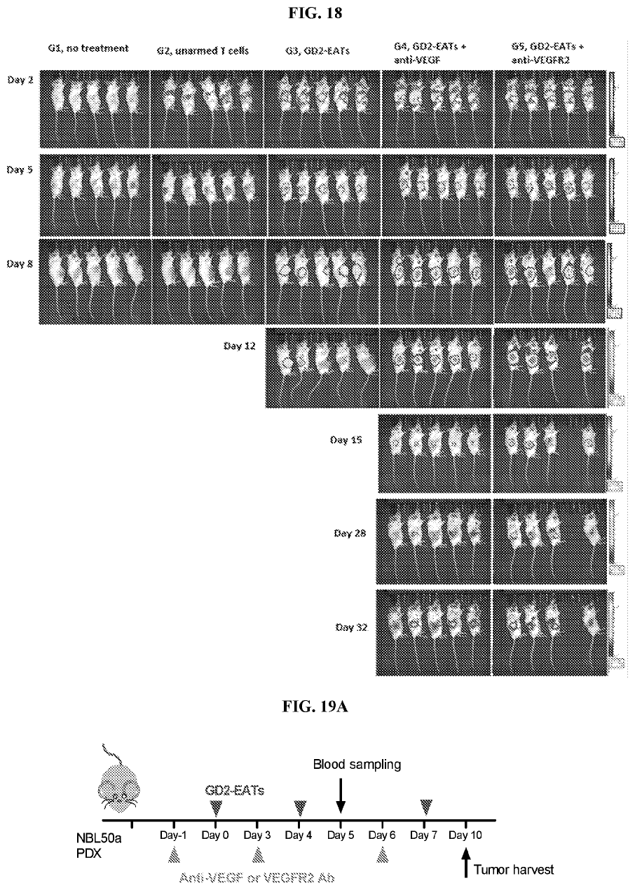

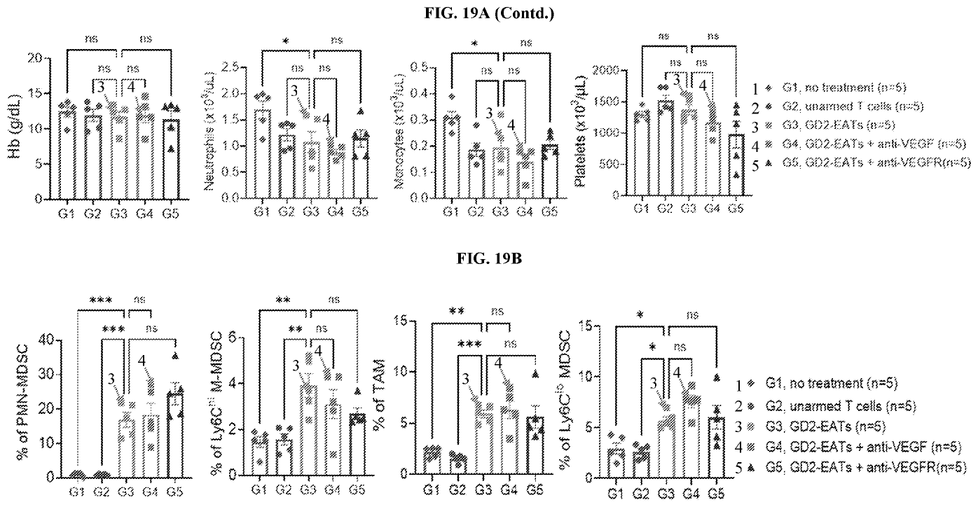

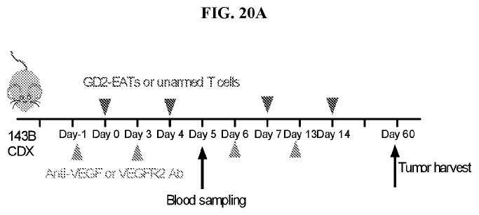

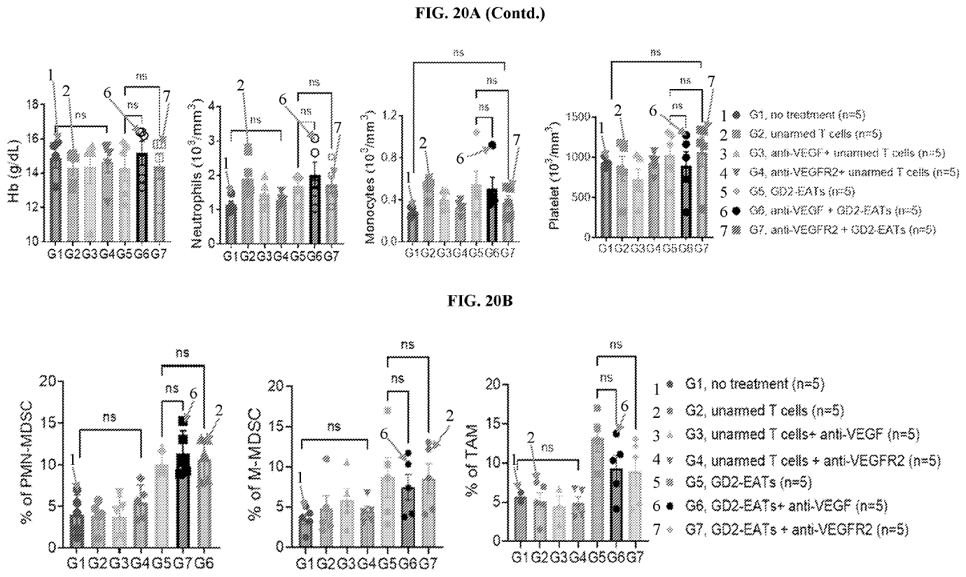

To test if VEGF blockades affected complete blood cell counts (CBC) or frequencies of tumor infiltrating myeloid cells (TIMs), CBC and flow cytometry analysis of tumors were done after the first and third doses of GD2-EATs following bevacizumab or DC101. There were no significant changes in the red blood cell, leukocyte, or platelet counts (FIG. 19A). The effect on TIMs was also analyzed using flow cytometry of tumors. As reported previously, while no treatment (G1) or unarmed T cells (G2) showed fewer TIMs, GD2-EATs (G3) attracted abundant mCD45(+) mCD11b(+) TIM. When VEGF blockade combination groups were compared (G4, GD2-EATs plus bevacizumab, and G5, GD2-EATs plus DC101), there was no significant difference in the frequencies of TIMs, whether among the Ly6G⁺ PMN-MDSC, Ly6C^{hi} M-MDSC, F4/80⁺ TAM, or Ly6C^{lo} MDSC subsets (FIG. 19B). TIM analysis in a second tumor model was repeated using 143B CDXs where blood and tumors were analyzed on day 5 and day 60 post treatment, respectively. Again, VEGF blockades did not induce significant difference in CBC among groups (FIG. 20A), and there were no differences in TIM frequencies including PMN-MDSC, M-MDSC, and TAM subsets (FIG. 20B).

### Effect of VEGF Blockades on Intratumoral Blood Vessels

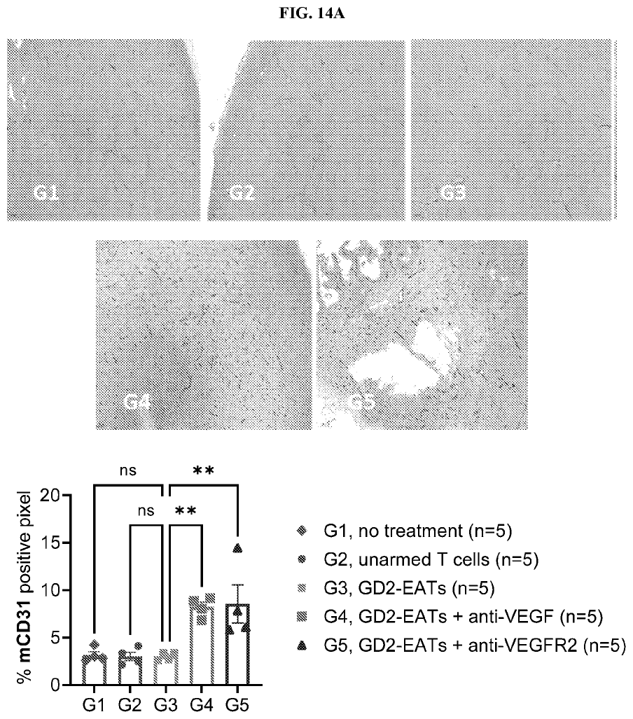

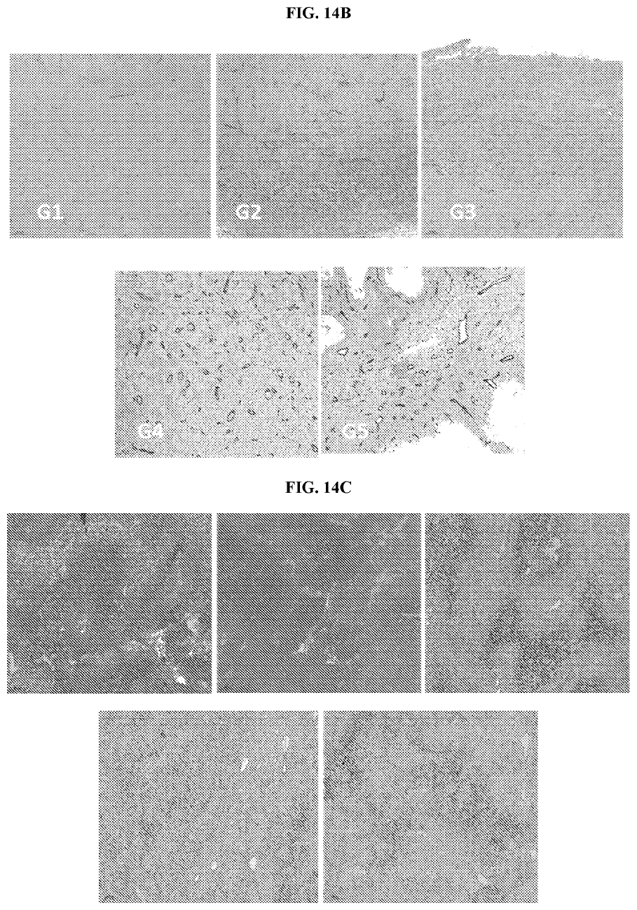

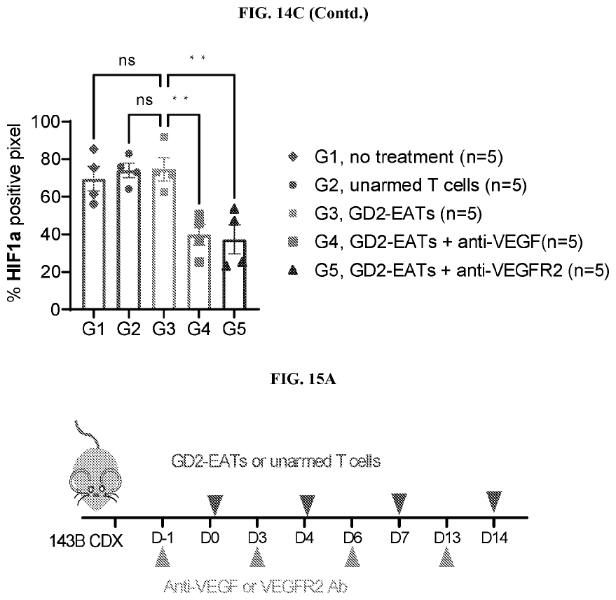

Intratumoral blood vessels in neuroblastoma PDXs were stained with anti-mCD31 antibody to study the effect of VEGF blockades on tumor microvasculature (FIG. 14A). Although there was no significant difference among the no treatment (G1), unarmed T cells (G2), and GD2-EATs (G3) groups, there was a significant increase in mCD31(+) blood vessels among groups treated with GD2-EATs plus VEGF blockade [G3 vs. G4 (+bevacizumab), p=0.0062; G3 vs. G5 (+DC101), p=0.0038] on day 10 post-treatment. Notably, VEGF blockades changed the shape of intratumoral blood vessels (FIG. 14B), appearing thickened with plump endothelial cells showing characteristic of high endothelial venules (HEVs), contrasting to vessels in the group of GD2-EATs alone presenting usual flat and thin endothelial cells typical of tumor microvasculature. These findings also correlated with HIF-1α staining results. Tumors treated by GD2-EATs with VEGF blockades displayed fewer areas of HIF-1α positivity when compared to those in no treatment, unarmed T cells, or GD2-EATs alone groups (FIG. 14C).

### VEGF Blockades Improved Tumor Responsiveness to EAT Therapy In Vivo

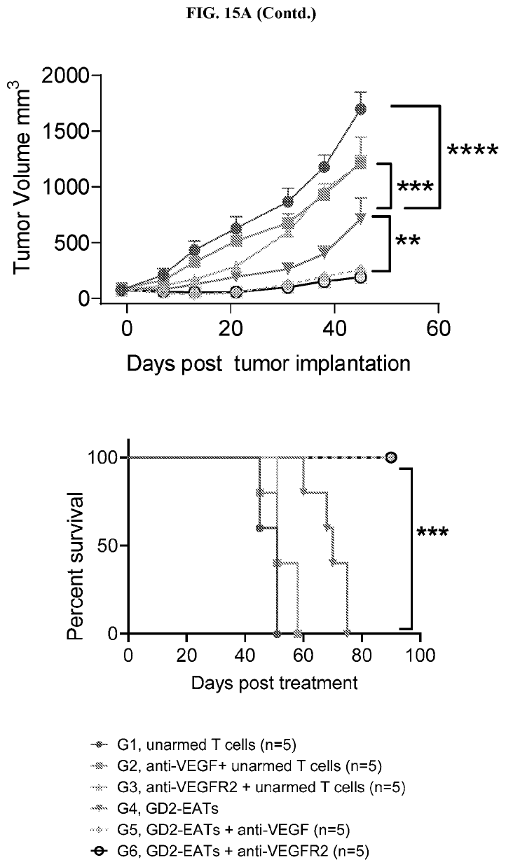

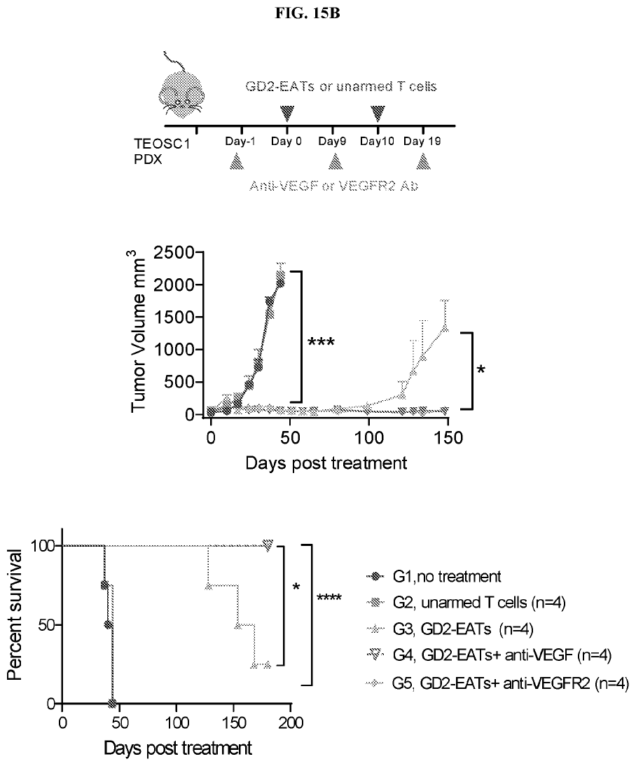

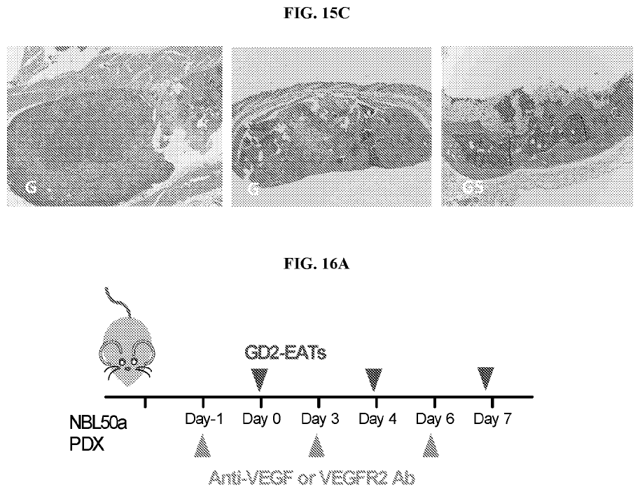

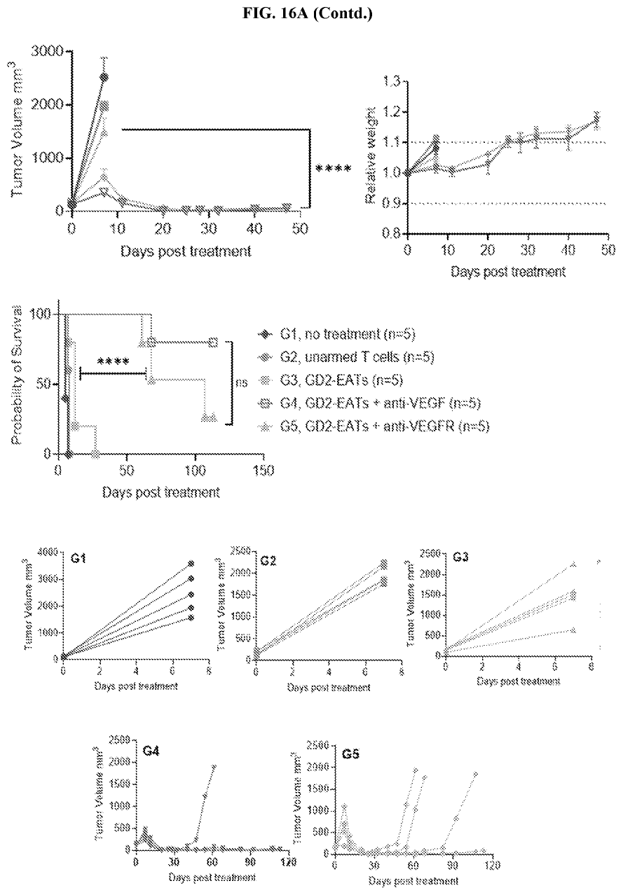

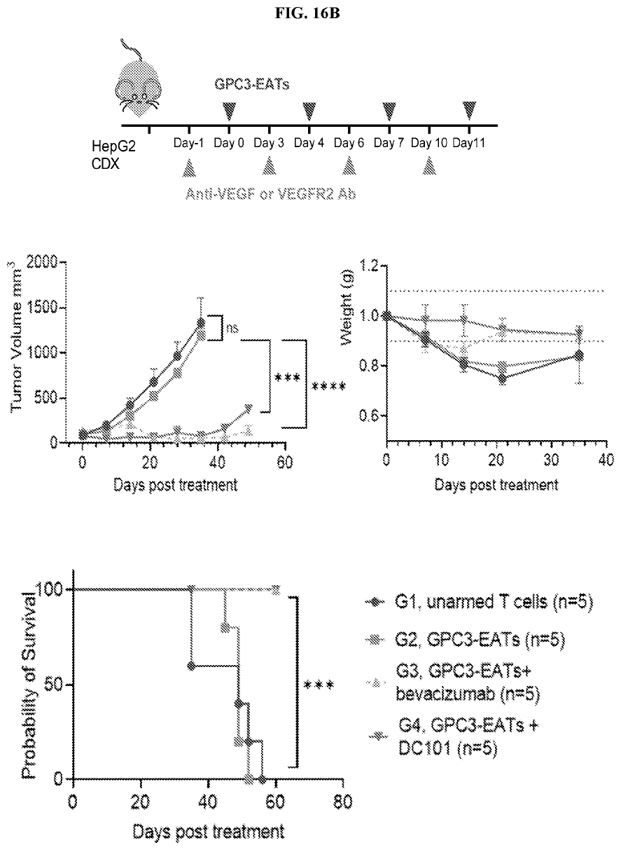

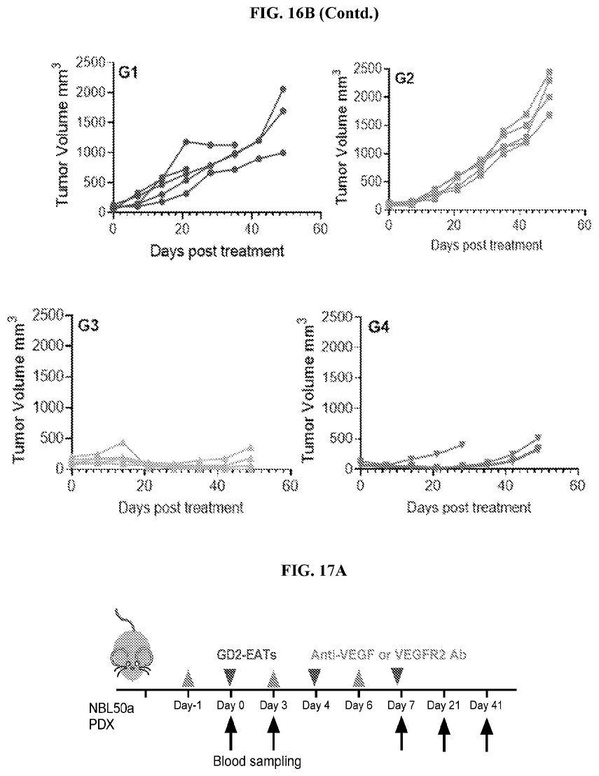

Next, the effect of VEGF blockades on in vivo anti-tumor response of EATs was studied. The effects in 4 different tumor models including two osteosarcomas, one neuroblastoma, and one hepatoblastoma were tested. First, osteosarcoma 143B CDXs were treated with GD2-EATs plus bevacizumab or DC101 (FIG. 15A). Treatment with bevacizumab or DC101 with unarmed T cells did not show any anti-tumor effect while GD2-EATs without VEGF blockades showed only modest tumor suppression. When bevacizumab or DC101 was added, the anti-tumor effect of GD2-EATs was significantly improved (P=0.0070 and P=0.0041, respectively), prolonging mouse survival (P=0.0002). In the second tumor model, TEOSC1 osteosarcoma PDXs were treated with 2 doses of GD2-EATs and 3 doses of either bevacizumab or DC101 (FIG. 15B). VEGF blockades plus GD2-EATs produced long-term remission past 6 months, significantly longer than GD2-EATs alone (P=0.0221). Of interest was the histologic changes among tumors treated by GD2-EATs plus VEGF blockades, showing fibrous and cavitary bone lesions characterized by intracavitary extramedullary hematopoiesis and mineralization, contrasting with relapsed tumors in GD2-EATs group showing chondroid differentiating osteosarcoma (FIG. 15C). In the third tumor model, neuroblastoma PDXs were treated with GD2-EATs in the presence of VEGF blockades. Contrary to the GD2-EATs alone group showing unabated tumor growth, GD2-EATs plus bevacizumab or plus DC101 presented significantly better anti-tumor effect in vivo (FIG. 16A). The combinations successfully ablated all tumors and sustained remission without clinical toxicity, significantly prolonging survival (P<0.0001). In the fourth model, HepG2 hepatoblastoma CDX was treated with T cells ex vivo armed with anti-glypican 3 BsAb (GPC3-EATs) with VEGF blockades (FIG. 16B). In contrast to unarmed T cells or GPC3-EATs plus control IgG, GPC3-EATs plus bevacizumab or plus DC101 significantly enhanced anti-tumor response, confirming the synergy between VEGF blockades and EATs.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating cancer or inhibiting tumor growth in a subject in need thereof comprising administering to the subject an effective amount of dexamethasone and an effective amount of an anti-CD3 multi-specific antibody,
wherein the anti-CD3 multi-specific antibody includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and
wherein the anti-CD3 multi-specific antibody is an immunoglobulin comprising two heavy chains and two light chains, wherein each of the light chains is fused to a single chain variable fragment (scFv), optionally wherein administration of dexamethasone and the anti-CD3 multi-specific antibody reduces or delays cytokine release syndrome (CRS) in the subject.

2. (canceled)

3. A method for treating cancer or ameliorating cytokine release syndrome in a subject in need thereof comprising A)
(a) administering to the subject a first effective dose of dexamethasone,
(b) administering to the subject a first effective amount of an anti-CD3 multi-specific antibody about 1 hour after administration of the first effective dose of dexamethasone,
(c) administering to the subject a second effective dose of dexamethasone about 72-96 hours after administration of the first effective dose of dexamethasone,
(d) administering to the subject a second effective amount of the anti-CD3 multi-specific antibody about 1 hour after administration of the second effective dose of dexamethasone, and
(e) repeating steps (a)-(d) for at least one additional cycle,
wherein the anti-CD3 multi-specific antibody includes a CD3 binding domain comprising a heavy chain immunoglobulin variable domain (V_{H}) and a light chain immunoglobulin variable domain (V_{L}), wherein
(i) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(ii) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and
wherein the anti-CD3 multi-specific antibody is an immunoglobulin comprising two heavy chains and two light chains, wherein each of the light chains is fused to a single chain variable fragment (scFv); or
(a) administering to the subject a first effective dose of dexamethasone,
(b) administering to the subject a first effective amount of an ex vivo armed T cell that is coated or complexed with an effective arming dose of at least one type of anti-CD3 multi-specific antibody about 1 hour after administration of the first effective dose of dexamethasone,
(c) administering to the subject a second effective dose of dexamethasone about 72-96 hours after administration of the first effective dose of dexamethasone,
(d) administering to the subject a second effective amount of the ex vivo armed T cell about 1 hour after administration of the second effective dose of dexamethasone, and
(e) repeating steps (a)-(d) for at least one additional cycle,
wherein the at least one type of anti-CD3 multi-specific antibody includes a CD3 binding domain comprising a heavy chain immunoglobulin variable domain (V_{H}) and a light chain immunoglobulin variable domain (V_{L}), wherein
(i) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(ii) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and
wherein the anti-CD3 multi-specific antibody is an immunoglobulin comprising two heavy chains and two light chains, wherein each of the light chains is fused to a single chain variable fragment (scFv).

4. (canceled)

5. The method of claim 3, wherein the first and/or second effective dose of dexamethasone is about 2 mg/kg to about 32 mg/kg or wherein the first second effective dose and second effective dose of dexamethasone are identical.

6. (canceled)

7. A method for treating cancer or inhibiting tumor growth or reducing or delaying cytokine release syndrome (CRS) in a subject in need thereof comprising administering to the subject an effective amount of dexamethasone and an effective amount of an ex vivo armed T cell that is coated or complexed with an effective arming dose of at least one type of anti-CD3 multi-specific antibody,
wherein the at least one type of anti-CD3 multi-specific antibody includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and
wherein the at least one type of anti-CD3 multi-specific antibody is an immunoglobulin comprising two heavy chains and two light chains, wherein each of the light chains is fused to a single chain variable fragment (scFv).

8. (canceled)

9. The method of claim 3, wherein the ex vivo armed T cell is or has been cryopreserved, optionally wherein the ex vivo armed T cell has been cryopreserved for a period of about 2 hours to about 6 months: or
wherein the ex vivo armed T cell is a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell: or
wherein the at least one type of anti-CD3 multi-specific antibody exhibits surface densities between about 500 to about 20,000 molecules per T cell: or
wherein the effective arming dose of the at least one type of anti-CD3 multi-specific antibody is between about 0.05 μg/10⁶ T cells to about 5 μg/10⁶ T cells: or
wherein the ex vivo armed T cell is autologous, non-autologous, or derived in vitro from lymphoid progenitor cells.

10. (canceled)

11. (canceled)

12. (canceled)

13. (canceled)

14. (canceled)

15. The method of claim 3, further comprising administering a cytokine to the subject, optionally wherein the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2, optionally wherein the cytokine is administered prior to, during, or subsequent to administration of the ex vivo armed T cell.

16. (canceled)

17. The method of claim 1, wherein at least one scFv of the anti-CD3 multi-specific antibody comprises the CD3 binding domain or wherein at least one scFv of the at least one type of anti-CD3 multi-specific antibody comprises the CD3 binding domain; or
wherein at least one scFv of the anti-CD3 multi-specific antibody comprises a DOTA binding domain or wherein at least one scFv of the at least one type of anti-CD3 multi-specific antibody comprises a DOTA binding domain: optionally wherein the DOTA binding domain comprises the amino acid sequence of any one of SEQ ID NOs: 77-80.

18. (canceled)

19. (canceled)

20. The method of claim 1, wherein the anti-CD3 multi-specific antibody or the at least one type of anti-CD3 multi-specific antibody binds one or more additional target antigens, optionally wherein the one or more additional target antigens are selected from the group consisting of CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), and a DOTA-based hapten.

21. (canceled)

22. The method of claim 1, wherein the V_{H} of the CD3 binding domain comprises the amino acid sequence of any one of SEQ ID NOs: 7-32, and/or wherein the V_{L} of the CD3 binding domain comprises the amino acid sequence of any one of SEQ ID NOs: 33-70.

23. The method of claim 1, wherein the anti-CD3 multi-specific antibody or the at least one type of anti-CD3 multi-specific antibody comprises a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, or a variant thereof having one or more conservative amino acid substitutions, and/or a light chain (LC) amino acid sequence comprising SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, or a variant thereof having one or more conservative amino acid substitutions.

24. The method of claim 23, wherein the anti-CD3 multi-specific antibody or the at least one type of anti-CD3 multi-specific antibody comprises a HC amino acid sequence and a LC amino acid sequence selected from the group consisting of:
SEQ ID NO: 82 and SEQ ID NO: 81,
SEQ ID NO: 84 and SEQ ID NO: 83,
SEQ ID NO: 86 and SEQ ID NO: 85,
SEQ ID NO: 88 and SEQ ID NO: 87,
SEQ ID NO: 90 and SEQ ID NO: 89,
SEQ ID NO: 92 and SEQ ID NO: 91,
SEQ ID NO: 94 and SEQ ID NO: 93,
SEQ ID NO: 96 and SEQ ID NO: 95,
SEQ ID NO: 98 and SEQ ID NO: 97,
SEQ ID NO: 100 and SEQ ID NO: 99,
SEQ ID NO: 102 and SEQ ID NO: 101,
SEQ ID NO: 104 and SEQ ID NO: 103,
SEQ ID NO: 106 and SEQ ID NO: 105,
SEQ ID NO: 108 and SEQ ID NO: 107,
SEQ ID NO: 110 and SEQ ID NO: 109,
SEQ ID NO: 112 and SEQ ID NO: 111,
SEQ ID NO: 114 and SEQ ID NO: 113,
SEQ ID NO: 116 and SEQ ID NO: 115,
SEQ ID NO: 118 and SEQ ID NO: 117,
SEQ ID NO: 120 and SEQ ID NO: 119,
SEQ ID NO: 122 and SEQ ID NO: 121,
SEQ ID NO: 124 and SEQ ID NO: 123,
SEQ ID NO: 126 and SEQ ID NO: 125,
SEQ ID NO: 128 and SEQ ID NO: 127,
SEQ ID NO: 130 and SEQ ID NO: 129,
SEQ ID NO: 132 and SEQ ID NO: 131,
SEQ ID NO: 134 and SEQ ID NO: 133,
SEQ ID NO: 136 and SEQ ID NO: 135,
SEQ ID NO: 138 and SEQ ID NO: 137,
SEQ ID NO: 140 and SEQ ID NO: 139,
SEQ ID NO: 142 and SEQ ID NO: 141,
SEQ ID NO: 144 and SEQ ID NO: 143,
SEQ ID NO: 146 and SEQ ID NO: 145,
SEQ ID NO: 148 and SEQ ID NO: 147, and
SEQ ID NO: 150 and SEQ ID NO: 149, respectively.

25. The method of claim 23, wherein the anti-CD3 multi-specific antibody or the at least one type of anti-CD3 multi-specific antibody comprise a first LC amino acid sequence, a first HC amino acid sequence, a second LC amino acid sequence, and a second HC amino acid sequence selected from the group consisting of
SEQ ID NO: 99, SEQ ID NO: 100, SEQ ID NO: 101, and SEQ ID NO: 102;
SEQ ID NO: 103, SEQ ID NO: 104, SEQ ID NO: 105, and SEQ ID NO: 106;
SEQ ID NO: 107, SEQ ID NO: 108, SEQ ID NO: 109, and SEQ ID NO: 110;
SEQ ID NO: 111, SEQ ID NO: 112, SEQ ID NO: 113, and SEQ ID NO: 114;
SEQ ID NO: 115, SEQ ID NO: 116, SEQ ID NO: 117, and SEQ ID NO: 118;
SEQ ID NO: 119, SEQ ID NO: 120, SEQ ID NO: 121, and SEQ ID NO: 122;
SEQ ID NO: 123, SEQ ID NO: 124, SEQ ID NO: 125, and SEQ ID NO: 126;
SEQ ID NO: 127, SEQ ID NO: 128, SEQ ID NO: 129, and SEQ ID NO: 130;
SEQ ID NO: 131, SEQ ID NO: 132, SEQ ID NO: 133, and SEQ ID NO: 134;
SEQ ID NO: 135, SEQ ID NO: 136, SEQ ID NO: 137, and SEQ ID NO: 138;
SEQ ID NO: 139, SEQ ID NO: 140, SEQ ID NO: 141, and SEQ ID NO: 142;
SEQ ID NO: 143, SEQ ID NO: 144, SEQ ID NO: 145, and SEQ ID NO: 146; and
SEQ ID NO: 147, SEQ ID NO: 148, SEQ ID NO: 149, and SEQ ID NO: 150; respectively.

26. The method of claim 1, wherein the dexamethasone, or the anti-CD3 multi-specific antibody is administered intravenously, intraperitoneally, subcutaneously, intramuscularly, or intratumorally.

27. The method of claim 1, wherein the ex vivo armed T cell is administered intravenously, intraperitoneally, subcutaneously, intramuscularly, or intratumorally.

28. The method of claim 1, wherein the dexamethasone is administered about one hour or about 24 hours prior to administration of the anti-CD3 multi-specific antibody.

29. The method of claim 1, wherein the dexamethasone is administered about one hour or about 24 hours prior to administration of the ex vivo armed T cell.

30. The method of claim 1, further comprising separately, simultaneously, or sequentially administering an additional cancer therapy, optionally wherein the additional cancer therapy is selected from among chemotherapy, radiation therapy, immunotherapy, monoclonal antibodies, anti-cancer nucleic acids or proteins, anti-cancer viruses or microorganisms, and any combinations thereof.

31. (canceled)

32. The method of claim 30, wherein the additional cancer therapy is an immune checkpoint inhibitor selected from among pembrolizumab, nivolumab, cemiplimab, atezolizumab, avelumab, durvalumab, and ipilimumab: or wherein the additional cancer therapy comprises one or more of an anti-Ly6G antibody, an anti-GR-1 antibody, an anti-Ly6C antibody, an anti-CSF-1R antibody, Clodronate, a VEGF inhibitor and a VEGFR inhibitor.

33. (canceled)

34. The method of claim 1, wherein the subject is diagnosed with, or is suspected of having cancer.

35. (canceled)

36. The method of claim 34, wherein the cancer is selected from the group consisting of sarcoma, hematopoietic cancer, osteosarcoma, Ewing's sarcoma, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof.


SEQUENCE LISTING (155 sequences)

SEQ ID NO: 1 - protein, 8 aa, Artificial Sequence
  Description of Artificial Sequence Synthetic peptide
GYTFTRYT

SEQ ID NO: 2 - protein, 8 aa, Artificial Sequence
  Description of Artificial Sequence Synthetic peptide
INPSRGYT

SEQ ID NO: 3 - protein, 12 aa, Artificial Sequence
  Description of Artificial Sequence Synthetic peptide
ARYYDDHYSLDY

SEQ ID NO: 4 - protein, 5 aa, Artificial Sequence
  Description of Artificial Sequence Synthetic peptide
SSVSY

SEQ ID NO: 5 - protein, 2 aa, Artificial Sequence
  Description of Artificial Sequence Synthetic peptide
DT

SEQ ID NO: 6 - protein, 9 aa, Artificial Sequence
  Description of Artificial Sequence Synthetic peptide
QQWSSNPFT

SEQ ID NO: 7 - protein, 119 aa, Artificial Sequence
  Description of Artificial Sequence Synthetic polypeptide
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 8 - protein, 119 aa, Artificial Sequence
  Description of Artificial Sequence Synthetic polypeptide
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 9 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLDYWGQGTTLTVSS

SEQ ID NO: 10 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSS

SEQ ID NO: 11 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLDYWGQGTTLTVSS

SEQ ID NO: 12 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLDYWGQGTTLTVSS

SEQ ID NO: 13 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLDYWGCGTTLTVSS

SEQ ID NO: 14 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGCGTTLTVSS

SEQ ID NO: 15 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLDYWGCGTTLTVSS

SEQ ID NO: 16 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLDYWGCGTTLTVSS

SEQ ID NO: 17 - protein, 119 aa, Artificial Sequence
  Description of Artificial Sequence Synthetic polypeptide
QVQLQQSGAEVAKPGASVKMSCKASGYTFTRYTMHWVRQAPGQCLEWIGYINPSRGYTNYAQKFQGRATLTTDKSISTAYMELSRLRSDDTAVYYCARYYDDHYSLDYWGQGTTLTVSS

SEQ ID NO: 18 - protein, 119 aa, Artificial Sequence
  Description of Artificial Sequence Synthetic polypeptide
QVQLQQSGAEVAKPGASVKVSCKASGYTFTRYTMHWVRQAPGQCLEWMGYINPSRGYTNYNQKFKDRATLTRDKSISTAYMELSRLRSDDTAVYYCARYYDDHYSLDYWGQGTTLTVSS

SEQ ID NO: 19 - protein, 119 aa, Artificial Sequence
  Description of Artificial Sequence Synthetic polypeptide
QVQLVQSGAEVKKPGASVKMSCKASGYTFTRYTMHWVRQAPGQCLEWIGYINPSRGYTNYNQKFKDRATMTTDKSISTAYMELSRLRSDDTAVYYCARYYDDHYSLDYWGQGTTLTVSS

SEQ ID NO: 20 - protein, 119 aa, Artificial Sequence
  Description of Artificial Sequence Synthetic polypeptide
QVQLQQSGAEVKKPGASVKMSCKASGYTFTRYTMHWVRQAPGQCLEWIGYINPSRGYTNYNQKFKDRVTLTTDTSISTAYMELSRLRSDDTAVYYCARYYDDHYSLDYWGQGTTLTVSS

SEQ ID NO: 21 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSCDYWGQGTTLTVSS

SEQ ID NO: 22 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSCDYWGQGTTLTVSS

SEQ ID NO: 23 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSCDYWGQGTTLTVSS

SEQ ID NO: 24 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSCDYWGQGTTLTVSS

SEQ ID NO: 25 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHCSLDYWGQGTTLTVSS

SEQ ID NO: 26 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHCSLDYWGQGTTLTVSS

SEQ ID NO: 27 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHCSLDYWGQGTTLTVSS

SEQ ID NO: 28 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHCSLDYWGQGTTLTVSS

SEQ ID NO: 29 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLCYWGQGTTLTVSS

SEQ ID NO: 30 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLCYWGQGTTLTVSS

SEQ ID NO: 31 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLCYWGQGTTLTVSS

SEQ ID NO: 32 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLCYWGQGTTLTVSS

SEQ ID NO: 33 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 34 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 35 - protein, 107 aa, Artificial Sequence
  Description of Artificial Sequence Synthetic polypeptide
DIQMTQSPSSLSASVGDRVTMTCSASSSVSYMNWYQQKPGKAPKRLIYDTSKLASGVPSRFSGSGSGTDFTLTISSLQPEDFATYYCQQWSSNPFTFGSGTKLEINR

SEQ ID NO: 36 - protein, 107 aa, Artificial Sequence
  Description of Artificial Sequence Synthetic polypeptide
DIQMTQSPSSLSASVGDRVTMTCSASSSVSYMNWYQQKPGKAPKLLIYDTSKLASGVPSRFSGSGSGTDFTLTISSMQPEDFATYYCQQWSSNPFTFGSGTKLEINR

SEQ ID NO: 37 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDYTLTISSLQPEDFATYYCQQWSSNPFTFGSGTKLEINR

SEQ ID NO: 38 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDFTLTISSMQPEDFATYYCQQWSSNPFTFGSGTKLEINR

SEQ ID NO: 39 - protein, 107 aa, Artificial Sequence
  Description of Artificial Sequence Synthetic polypeptide
DIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 40 - protein, 107 aa, Artificial Sequence
  Description of Artificial Sequence Synthetic polypeptide
DIQMTQSPSSLSASVGDRVTITCSASSSVSYMNWYQQKPGKAPKLWIYDTSKLASGVPSRFSGSGSGTDYTLTISSLQPEDFATYYCQQWSSNPFTFGSGTKLEINR

SEQ ID NO: 41 - protein, 107 aa, Artificial Sequence
  Description of Artificial Sequence Synthetic polypeptide
DIQMTQSPSSLSASVGDRVTMTCSASSSVSYMNWYQQKPGKAPKRLIYDTSKLASGVPSRFSGSGSGTDFTLTISSLQPEDFATYYCQQWSSNPFTFGCGTKLEINR

SEQ ID NO: 42 - protein, 107 aa, Artificial Sequence
  Description of Artificial Sequence Synthetic polypeptide
DIQMTQSPSSLSASVGDRVTMTCSASSSVSYMNWYQQKPGKAPKLLIYDTSKLASGVPSRFSGSGSGTDFTLTISSMQPEDFATYYCQQWSSNPFTFGCGTKLEINR

SEQ ID NO: 43 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDYTLTISSLQPEDFATYYCQQWSSNPFTFGCGTKLEINR

SEQ ID NO: 44 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDFTLTISSMQPEDFATYYCQQWSSNPFTFGCGTKLEINR

SEQ ID NO: 45 - protein, 107 aa, Artificial Sequence
  Description of Artificial Sequence Synthetic polypeptide
DIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGCGTKLEINR

SEQ ID NO: 46 - protein, 107 aa, Artificial Sequence
  Description of Artificial Sequence Synthetic polypeptide
DIQMTQSPSSLSASVGDRVTITCSASSSVSYMNWYQQKPGKAPKLWIYDTSKLASGVPSRFSGSGSGTDYTLTISSLQPEDFATYYCQQWSSNPFTFGCGTKLEINR

SEQ ID NO: 47 - protein, 107 aa, Artificial Sequence
  Description of Artificial Sequence Synthetic polypeptide
DIQMTQSPSSLSASVGDRVTMTCSASSSVSYMNWYQQKPGKCPKRLIYDTSKLASGVPSRFSGSGSGTDFTLTISSLQPEDFATYYCQQWSSNPFTFGSGTKLEINR

SEQ ID NO: 48 - protein, 107 aa, Artificial Sequence
  Description of Artificial Sequence Synthetic polypeptide
DIQMTQSPSSLSASVGDRVTMTCSASSSVSYMNWYQQKPGKCPKLLIYDTSKLASGVPSRFSGSGSGTDFTLTISSMQPEDFATYYCQQWSSNPFTFGSGTKLEINR

SEQ ID NO: 49 - protein, 107 aa, Artificial Sequence
  Description of Artificial Sequence Synthetic polypeptide
DIQLTQSPSSLSASVGDRVTITCSASSSVSYMNWYQQKPGKCPKLLIYDTSKLASGVPSRFSGSGSGTDYTLTISSLQPEDFATYYCQQWSSNPFTFGSGTKLEINR

SEQ ID NO: 50 - protein, 107 aa, Artificial Sequence
  Description of Artificial Sequence Synthetic polypeptide
DIQLTQSPSSLSASVGDRVTITCSASSSVSYMNWYQQKPGKCPKLLIYDTSKLASGVPSRFSGSGSGTDFTLTISSMQPEDFATYYCQQWSSNPFTFGSGTKLEINR

SEQ ID NO: 51 - protein, 107 aa, Artificial Sequence
  Description of Artificial Sequence Synthetic polypeptide
DIQMTQSPSSLSASVGDRVTITCSASSSVSYMNWYQQKPGKCPKRWIYDTSKLASGVPSRFSGSGSGTDFTLTISSLQPEDFATYYCQQWSSNPFTFGSGTKLEINR

SEQ ID NO: 52 - protein, 107 aa, Artificial Sequence
  Description of Artificial Sequence Synthetic polypeptide
DIQMTQSPSSLSASVGDRVTITCSASSSVSYMNWYQQKPGKCPKLWIYDTSKLASGVPSRFSGSGSGTDYTLTISSLQPEDFATYYCQQWSSNPFTFGSGTKLEINR

SEQ ID NO: 53 - protein, 107 aa, Artificial Sequence
  Description of Artificial Sequence Synthetic polypeptide
DIQMTQSPSSLSASVGDRVTMTCSASSSVSYMNWYQQKPGKAPKRLICDTSKLASGVPSRFSGSGSGTDFTLTISSLQPEDFATYYCQQWSSNPFTFGSGTKLEINR

SEQ ID NO: 54 - protein, 107 aa, Artificial Sequence
  Description of Artificial Sequence Synthetic polypeptide
DIQMTQSPSSLSASVGDRVTMTCSASSSVSYMNWYQQKPGKAPKLLICDTSKLASGVPSRFSGSGSGTDFTLTISSMQPEDFATYYCQQWSSNPFTFGSGTKLEINR

SEQ ID NO: 55 - protein, 107 aa, Artificial Sequence
  Description of Artificial Sequence Synthetic polypeptide
DIQLTQSPSSLSASVGDRVTITCSASSSVSYMNWYQQKPGKAPKLLICDTSKLASGVPSRFSGSGSGTDYTLTISSLQPEDFATYYCQQWSSNPFTFGSGTKLEINR

SEQ ID NO: 56 - protein, 107 aa, Artificial Sequence
  Description of Artificial Sequence Synthetic polypeptide
DIQLTQSPSSLSASVGDRVTITCSASSSVSYMNWYQQKPGKAPKLLICDTSKLASGVPSRFSGSGSGTDFTLTISSMQPEDFATYYCQQWSSNPFTFGSGTKLEINR

SEQ ID NO: 57 - protein, 107 aa, Artificial Sequence
  Description of Artificial Sequence Synthetic polypeptide
DIQMTQSPSSLSASVGDRVTITCSASSSVSYMNWYQQKPGKAPKRWICDTSKLASGVPSRFSGSGSGTDFTLTISSLQPEDFATYYCQQWSSNPFTFGSGTKLEINR

SEQ ID NO: 58 - protein, 107 aa, Artificial Sequence
  Description of Artificial Sequence Synthetic polypeptide
DIQMTQSPSSLSASVGDRVTITCSASSSVSYMNWYQQKPGKAPKLWICDTSKLASGVPSRFSGSGSGTDYTLTISSLQPEDFATYYCQQWSSNPFTFGSGTKLEINR

SEQ ID NO: 59 - protein, 107 aa, Artificial Sequence
  Description of Artificial Sequence Synthetic polypeptide
DIQMTQSPSSLSASVGDRVTMTCSASSSVSYMNWYQQKPGKAPKRLIYCTSKLASGVPSRFSGSGSGTDFTLTISSLQPEDFATYYCQQWSSNPFTFGSGTKLEINR

SEQ ID NO: 60 - protein, 107 aa, Artificial Sequence
  Description of Artificial Sequence Synthetic polypeptide
DIQMTQSPSSLSASVGDRVTMTCSASSSVSYMNWYQQKPGKAPKLLIYCTSKLASGVPSRFSGSGSGTDFTLTISSMQPEDFATYYCQQWSSNPFTFGSGTKLEINR

SEQ ID NO: 61 - protein, 107 aa, Artificial Sequence
  Description of Artificial Sequence Synthetic polypeptide
DIQLTQSPSSLSASVGDRVTITCSASSSVSYMNWYQQKPGKAPKLLIYCTSKLASGVPSRFSGSGSGTDYTLTISSLQPEDFATYYCQQWSSNPFTFGSGTKLEINR

SEQ ID NO: 62 - protein, 107 aa, Artificial Sequence
  Description of Artificial Sequence Synthetic polypeptide
DIQLTQSPSSLSASVGDRVTITCSASSSVSYMNWYQQKPGKAPKLLIYCTSKLASGVPSRFSGSGSGTDFTLTISSMQPEDFATYYCQQWSSNPFTFGSGTKLEINR

SEQ ID NO: 63 - protein, 107 aa, Artificial Sequence
  Description of Artificial Sequence Synthetic polypeptide
DIQMTQSPSSLSASVGDRVTITCSASSSVSYMNWYQQKPGKAPKRWIYCTSKLASGVPSRFSGSGSGTDFTLTISSLQPEDFATYYCQQWSSNPFTFGSGTKLEINR

SEQ ID NO: 64 - protein, 107 aa, Artificial Sequence
  Description of Artificial Sequence Synthetic polypeptide
DIQMTQSPSSLSASVGDRVTITCSASSSVSYMNWYQQKPGKAPKLWIYCTSKLASGVPSRFSGSGSGTDYTLTISSLQPEDFATYYCQQWSSNPFTFGSGTKLEINR

SEQ ID NO: 65 - protein, 107 aa, Artificial Sequence
  Description of Artificial Sequence Synthetic polypeptide
DIQMTQSPSSLSASVGDRVTMTCSASSSVSYMNWYQQKPGKAPKCLIYDTSKLASGVPSRFSGSGSGTDFTLTISSLQPEDFATYYCQQWSSNPFTFGSGTKLEINR

SEQ ID NO: 66 - protein, 107 aa, Artificial Sequence
  Description of Artificial Sequence Synthetic polypeptide
DIQMTQSPSSLSASVGDRVTMTCSASSSVSYMNWYQQKPGKAPKCLIYDTSKLASGVPSRFSGSGSGTDFTLTISSMQPEDFATYYCQQWSSNPFTFGSGTKLEINR

SEQ ID NO: 67 - protein, 107 aa, Artificial Sequence
  Description of Artificial Sequence Synthetic polypeptide
DIQLTQSPSSLSASVGDRVTITCSASSSVSYMNWYQQKPGKAPKCLIYDTSKLASGVPSRFSGSGSGTDYTLTISSLQPEDFATYYCQQWSSNPFTFGSGTKLEINR

SEQ ID NO: 68 - protein, 107 aa, Artificial Sequence
  Description of Artificial Sequence Synthetic polypeptide
DIQLTQSPSSLSASVGDRVTITCSASSSVSYMNWYQQKPGKAPKCLIYDTSKLASGVPSRFSGSGSGTDFTLTISSMQPEDFATYYCQQWSSNPFTFGSGTKLEINR

SEQ ID NO: 69 - protein, 107 aa, Artificial Sequence
  Description of Artificial Sequence Synthetic polypeptide
DIQMTQSPSSLSASVGDRVTITCSASSSVSYMNWYQQKPGKAPKCWIYDTSKLASGVPSRFSGSGSGTDFTLTISSLQPEDFATYYCQQWSSNPFTFGSGTKLEINR

SEQ ID NO: 70 - protein, 107 aa, Artificial Sequence
  Description of Artificial Sequence Synthetic polypeptide
DIQMTQSPSSLSASVGDRVTITCSASSSVSYMNWYQQKPGKAPKCWIYDTSKLASGVPSRFSGSGSGTDYTLTISSLQPEDFATYYCQQWSSNPFTFGSGTKLEINR

SEQ ID NO: 71 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 72 - protein, 330 aa, Homo sapiens [sequence omitted]

SEQ ID NO: 73 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 74 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 75 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 119 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 78 - protein, 110 aa, Artificial Sequence
  Description of Artificial Sequence Synthetic polypeptide
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 79 - protein, 244 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 80 - protein, 259 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 81 - protein, 484 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 82 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 84 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 487 aa, Artificial Sequence
  Description of Artificial Sequence Synthetic polypeptide
DIQMTQSQSSLSTSVGDRVTITCKASQNVRTVVAWYQQKPGKSPKTLIYLASNRHTGVPSRFSGSGSGTEFTLTISNVQPEDFADYFCLQHWSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 86 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 493 aa, Artificial Sequence
  Description of Artificial Sequence Synthetic polypeptide
DIVMTQSPDSLAVSVGERVTMNCKSSQSLLYRSNQKNYLAWYQQKPGQPPKLLIYWASTRESGVPDRFSGSGSGTDFTLTISSVQAEDVAVYYCQQYYNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 88 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 491 aa, Artificial Sequence
  Description of Artificial Sequence Synthetic polypeptide
EIVLTQSPATLSVSLGERATISCRASESVDNYGISFMNWFQQKPGQPPRLLIYAASNQGSGVPARFSGSGPGTDFTLTISSMEPEDFAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 90 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 487 aa, Artificial Sequence
  Description of Artificial Sequence Synthetic polypeptide
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 92 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 487 aa, Artificial Sequence
  Description of Artificial Sequence Synthetic polypeptide
EIVLTQSPATLSVSPGETVTLSCRASQTIYKNLHWYQQKSGLSPRLLIKYGSDSISGIPARFSGSGSGTDYTLTINSVEPEDEGIYYCLQGYSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 94 - protein, 443 aa, Artificial Sequence
  Description of Artificial Sequence Synthetic polypeptide
QVQLVQSGPEVKKPGASVKISCKASGYTFTDYSMHWVKKAPGQGLKWLGWINTATGEPTYADDFKGRFTITLDTSARTVYLEISSLRSEDTATYFCFSYYDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 493 aa, Artificial Sequence
  Description of Artificial Sequence Synthetic polypeptide
DIVMSQSPSSLAVSVGEKVTMSCKSSQSLLYSSNQKNYLAWYQQKPGQSPKLLIYWASTRESGVPDRFTGSGSGTDFTLTISSVKAEDLALYYCQQYHSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 96 - protein, 448 aa, Artificial Sequence
  Description of Artificial Sequence Synthetic polypeptide
QVQLVQPGDELVKPGASVKLSCKASGYTFTSYWMQWVKQRPGQGLEWIGEINPSNGRTNYNEMFKSKAVLSVDKSVSTAYMQLSSLTAEDTAVYYCALYDGYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 492 aa, Artificial Sequence
  Description of Artificial Sequence Synthetic polypeptide
DIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 98 - protein, 449 aa, Artificial Sequence
  Description of Artificial Sequence Synthetic polypeptide
EVQLVESGGGVVQPGRSLRLSCSTSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 484 aa, Artificial Sequence; identical to SEQ ID NO: 81
  Description of Artificial Sequence Synthetic polypeptide

SEQ ID NO: 100 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 101 - protein, 487 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 102 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 103 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 104 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 105 - protein, 490 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 106 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 107 - protein, 493 aa, Artificial Sequence
  Description of Artificial Sequence Synthetic polypeptide
DIVMTQTPPSLPVNPGEPASISCRSSQSLVHRNGNTYLHWYLQKPGQSPQLLIHKVSNRFSGVPDRFSGSGSGSDFTLKISWVEAEDVGVYYCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 108 - protein, 443 aa, Artificial Sequence
  Description of Artificial Sequence Synthetic polypeptide
QVQLLQSGAEVKKPGASVKISCKASGSSFTGYNMNWVRQAPGQGLEWIGAIDPYYGGTSYNQKFKGRATLTVDKSISTAYMELSRLRSDDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 109 - protein, 496 aa, Artificial Sequence
  Description of Artificial Sequence Synthetic polypeptide
DIVMTQTPPSLPVNPGEPASISCRSSQSLVHRNGNTYLHWYLQKPGQSPQLLIHKVSNRFSGVPDRFSGSGSGSDFTLKISWVEAEDVGVYYCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 110 - protein, 443 aa, Artificial Sequence
  Description of Artificial Sequence Synthetic polypeptide
QVQLLQSGAEVKKPGASVKISCKASGSSFTGYNMNWVRQAPGQGLEWIGAIDPYYGGTSYNQKFKGRATLTVDKSISTAYMELSRLRSDDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 111 - protein, 487 aa, Artificial Sequence; identical to SEQ ID NO: 85
  Description of Artificial Sequence Synthetic polypeptide

SEQ ID NO: 112 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 113 - protein, 490 aa, Artificial Sequence
  Description of Artificial Sequence Synthetic polypeptide
DIQMTQSQSSLSTSVGDRVTITCKASQNVRTVVAWYQQKPGKSPKTLIYLASNRHTGVPSRFSGSGSGTEFTLTISNVQPEDFADYFCLQHWSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 114 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 115 - protein, 493 aa, Artificial Sequence
  Description of Artificial Sequence Synthetic polypeptide
DIVMTQSPSSLVVSIGERVTMNCKSSQSLLYSSNQKNYLAWYQQKPGQSPKLLIYWASSRESGVPDRFSGSGSGTDFTLTISSVKAEDVAVYYCQQYYNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 116 - protein, 447 aa, Artificial Sequence
  Description of Artificial Sequence Synthetic polypeptide
EVQLVESGGGLVQPEGSLKLSCAASGFTFNKNAMNWVRQAPGKGLEWVARIRNKTNNYATYYADSVKARFTISRDDSQSMLYLQMNSLKIEDTAMYYCVAGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 117 - protein, 496 aa, Artificial Sequence
  Description of Artificial Sequence Synthetic polypeptide
DIVMTQSPSSLVVSIGERVTMNCKSSQSLLYSSNQKNYLAWYQQKPGQSPKLLIYWASSRESGVPDRFSGSGSGTDFTLTISSVKAEDVAVYYCQQYYNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 118 - protein, 447 aa, Artificial Sequence
  Description of Artificial Sequence Synthetic polypeptide
EVQLVESGGGLVQPEGSLKLSCAASGFTFNKNAMNWVRQAPGKGLEWVARIRNKTNNYATYYADSVKARFTISRDDSQSMLYLQMNSLKIEDTAMYYCVAGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 119 - protein, 493 aa, Artificial Sequence; identical to SEQ ID NO: 87
  Description of Artificial Sequence Synthetic polypeptide

SEQ ID NO: 120 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 121 - protein, 496 aa, Artificial Sequence
  Description of Artificial Sequence Synthetic polypeptide
DIVMTQSPDSLAVSVGERVTMNCKSSQSLLYRSNQKNYLAWYQQKPGQPPKLLIYWASTRESGVPDRFSGSGSGTDFTLTISSVQAEDVAVYYCQQYYNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 122 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 123 - protein, 487 aa, Artificial Sequence
  Description of Artificial Sequence Synthetic polypeptide
DIQMTQSPSSLSASVGDRVTITCQASQDISKYLNWYQQKPGKAVKLLIYHTSRLHSGVPSRFSGSGSGTDYTLTISSLQPEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSSGGGGSGGGGSGGGGSGGGGSGGGGSGGGGSDIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 124 - protein, 450 aa, Artificial Sequence
  Description of Artificial Sequence Synthetic polypeptide
QVQLQESGPGLVKPSETLSVTCTVSGVSLPDYGVSWIRQPPGKGLEWIGVIWGSETTYYNPSLKSRVTISVDTSKNQVSLKLSSVTAADTAVYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 125 - protein, 490 aa, Artificial Sequence
  Description of Artificial Sequence Synthetic polypeptide
DIQMTQSPSSLSASVGDRVTITCQASQDISKYLNWYQQKPGKAVKLLIYHTSRLHSGVPSRFSGSGSGTDYTLTISSLQPEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 126 - protein, 450 aa, Artificial Sequence
  Description of Artificial Sequence Synthetic polypeptide
QVQLQESGPGLVKPSETLSVTCTVSGVSLPDYGVSWIRQPPGKGLEWIGVIWGSETTYYNPSLKSRVTISVDTSKNQVSLKLSSVTAADTAVYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 127 - protein, 491 aa, Artificial Sequence; identical to SEQ ID NO: 89
  Description of Artificial Sequence Synthetic polypeptide

SEQ ID NO: 128 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 129 - protein, 494 aa, Artificial Sequence
  Description of Artificial Sequence Synthetic polypeptide
EIVLTQSPATLSVSLGERATISCRASESVDNYGISFMNWFQQKPGQPPRLLIYAASNQGSGVPARFSGSGPGTDFTLTISSMEPEDFAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 130 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 131 - protein, 486 aa, Artificial Sequence
  Description of Artificial Sequence Synthetic polypeptide
DIQMTQSPSSLSASVGDRVTITCKASQDINKYIAWYQHKPGKAPKLLIYYASNLQPGVPSRFSGSGSGRDFTFTISSLQPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSSGGGGSGGGGSGGGGSGGGGSGGGGSGGGGSDIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 132 - protein, 449 aa, Artificial Sequence
  Description of Artificial Sequence Synthetic polypeptide
QVQLQQSGAEVVKPGASVKVSCKASGYSFTDYNMYWVRQAPGQGLEWMGYIDPYKGGTIYNQKFQGRATLTRDTSISTAYMELSRLRSDDTAVYYCAREMITAYYFDYWGQGSSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 133 - protein, 489 aa, Artificial Sequence
  Description of Artificial Sequence Synthetic polypeptide
DIQMTQSPSSLSASVGDRVTITCKASQDINKYIAWYQHKPGKAPKLLIYYASNLQPGVPSRFSGSGSGRDFTFTISSLQPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 134 - protein, 449 aa, Artificial Sequence
  Description of Artificial Sequence Synthetic polypeptide
QVQLQQSGAEVVKPGASVKVSCKASGYSFTDYNMYWVRQAPGQGLEWMGYIDPYKGGTIYNQKFQGRATLTRDTSISTAYMELSRLRSDDTAVYYCAREMITAYYFDYWGQGSSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 135 - protein, 492 aa, Artificial Sequence
  Description of Artificial Sequence Synthetic polypeptide
DIVMTQSPDSLAVSLGERATINCKSSQSVFFSSSQKNYLAWYQQKPGQPPKLLIYWASTRESGVPDRFSGSGSGTDFTLTISSLQAEDVAVYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 136 - protein, 448 aa, Artificial Sequence
  Description of Artificial Sequence Synthetic polypeptide
QVQLVQSGAEVKKPGASVKVSCKASGYTFTSYYIHWVRQAPGQGLEWMGVIYPGNDDISYAQKFQGRVTITADKSATTAYMELSSLRSEDTAVYYCAREVRLRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 137 - protein, 495 aa, Artificial Sequence
  Description of Artificial Sequence Synthetic polypeptide
DIVMTQSPDSLAVSLGERATINCKSSQSVFFSSSQKNYLAWYQQKPGQPPKLLIYWASTRESGVPDRFSGSGSGTDFTLTISSLQAEDVAVYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 138 - protein, 448 aa, Artificial Sequence
  Description of Artificial Sequence Synthetic polypeptide
QVQLVQSGAEVKKPGASVKVSCKASGYTFTSYYIHWVRQAPGQGLEWMGVIYPGNDDISYAQKFQGRVTITADKSATTAYMELSSLRSEDTAVYYCAREVRLRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 139 - protein, 487 aa, Artificial Sequence; identical to SEQ ID NO: 91
  Description of Artificial Sequence Synthetic polypeptide

SEQ ID NO: 140 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 141 - protein, 490 aa, Artificial Sequence
  Description of Artificial Sequence Synthetic polypeptide
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 142 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 143 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 144 - protein, 450 aa, Artificial Sequence; identical to SEQ ID NO: 104
  Description of Artificial Sequence Synthetic polypeptide

SEQ ID NO: 145 - protein, 490 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 146 - protein, 450 aa, Artificial Sequence; identical to SEQ ID NO: 106
  Description of Artificial Sequence Synthetic polypeptide

SEQ ID NO: 147 - protein, 486 aa, Artificial Sequence
  Description of Artificial Sequence Synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 148 - protein, 449 aa, Artificial Sequence
  Description of Artificial Sequence Synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 149 - protein, 489 aa, Artificial Sequence
  Description of Artificial Sequence Synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 150 - protein, 449 aa, Artificial Sequence
  Description of Artificial Sequence Synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 151 - protein, 20 aa, Artificial Sequence
  Description of Artificial Sequence Synthetic peptide
GGGGSGGGGSGGGGSGGGGS

SEQ ID NO: 152 - protein, 25 aa, Artificial Sequence
  Description of Artificial Sequence Synthetic peptide
GGGGSGGGGSGGGGSGGGGSGGGGS

SEQ ID NO: 153 - protein, 30 aa, Artificial Sequence
  Description of Artificial Sequence Synthetic polypeptide
GGGGSGGGGSGGGGSGGGGSGGGGSGGGGS

SEQ ID NO: 154 - protein, 15 aa, Artificial Sequence
  Description of Artificial Sequence Synthetic peptide
GGGGSGGGGSGGGGS

SEQ ID NO: 155 - protein, 5 aa, Artificial Sequence
  Description of Artificial Sequence Synthetic peptide
  See specification as filed for detailed description of substitutions and preferred embodiments
GGGGS